SEQUENCE LISTING (16 sequences)

SEQ ID NO: 1 - DNA, 20 bp, Artificial Sequence
  /note=“Description of Artificial Sequence Synthetic primer sequence”
cccgagtatctggaagacag

SEQ ID NO: 2 - DNA, 17 bp, Artificial Sequence
  /note=“Description of Artificial Sequence Synthetic primer sequence”
ataggtcggcggttcat

SEQ ID NO: 3 - DNA, 23 bp, Artificial Sequence
  /note=“Description of Artificial Sequence Synthetic primer sequence”
tgaagactgctcgcagagtgata

SEQ ID NO: 4 - DNA, 21 bp, Artificial Sequence
  /note=“Description of Artificial Sequence Synthetic primer sequence”
agcttccaggtgagccatttc

SEQ ID NO: 5 - DNA, 24 bp, Artificial Sequence
  /note=“Description of Artificial Sequence Synthetic primer sequence”
cagaggaggaacgagctgaagcgc

SEQ ID NO: 6 - DNA, 28 bp, Artificial Sequence
  /note=“Description of Artificial Sequence Synthetic primer sequence”
ttatgcaccagagtttcgaagctgttcg

SEQ ID NO: 7 - DNA, 20 bp, Artificial Sequence
  /note=“Description of Artificial Sequence Synthetic primer sequence”
agaccataacccaccacagc

SEQ ID NO: 8 - DNA, 20 bp, Artificial Sequence
  /note=“Description of Artificial Sequence Synthetic primer sequence”
tagggcctcttgtgccttta

SEQ ID NO: 9 - DNA, 22 bp, Artificial Sequence
  /note=“Description of Artificial Sequence Synthetic primer sequence”
tccgagcagagatcttcaggaa

SEQ ID NO: 10 - DNA, 22 bp, Artificial Sequence
  /note=“Description of Artificial Sequence Synthetic primer sequence”
tgcaaccaccactcattctgag

SEQ ID NO: 11 - DNA, 19 bp, Artificial Sequence
  /note=“Description of Artificial Sequence Synthetic primer sequence”
gccgtcattttctgcctca

SEQ ID NO: 12 - DNA, 19 bp, Artificial Sequence
  /note=“Description of Artificial Sequence Synthetic primer sequence”
cgtccttgcgagagggatc

SEQ ID NO: 13 - DNA, 19 bp, Artificial Sequence
  /note=“Description of Artificial Sequence Synthetic primer sequence”
cttagagggacaagtggcg

SEQ ID NO: 14 - DNA, 20 bp, Artificial Sequence
  /note=“Description of Artificial Sequence Synthetic primer sequence”
acgctgagccagtcagtgta

SEQ ID NO: 15 - DNA, 20 bp, Artificial Sequence
  /note=“Description of Artificial Sequence Synthetic primer sequence”
acaactttggcattgtggaa

SEQ ID NO: 16 - DNA, 20 bp, Artificial Sequence
  /note=“Description of Artificial Sequence Synthetic primer sequence”
gatgcagggatgatgttctg

(19) United States
(12) Patent Application Publication
(10) Pub. No.: US 2020/0268864 A1
(43) Pub. Date: Aug. 27, 2020

(54) CANCER VACCINE COMPOSITIONS AND METHODS FOR USING SAME TO TREAT CANCER

(71) Applicants: Jean ZHAO, Brookline, MA (US); Liya DING, Chestnut Hill, MA (US); Oiwei WANG, Boston, MA (US); Hye-Jung KIM, Brookline, MA (US); Dana-Farber Cancer Institute, Inc., Boston, MA (US)

(72) Inventors: Jean Zhao, Brookline, MA (US); Liya Ding, Chestnut Hill, MA (US); Qiwei Wang, Boston, MA (US); Hye-Jung Kim, Brookline, MA (US)

(21) Appl. No.: 16/755,636

(22) PCT Filed: Oct. 24, 2018
(86) PCT No.: PCT/US18/57213

§ 371 Date: Apr. 13, 2020

Related U.S. Application Data
(60) Provisional application No. 62/577,026, filed on Oct. 25, 2017.

(51) Int. Cl. A61K 39/00; A61K 39/39

(52) U.S. Cl. CPC A61K 39/001152; A61K 39/001148; A61K 45/06; A61K 39/39; A61K 39/001156

(57) ABSTRACT

The present invention provides a cancer vaccine comprising DNA repair-deficient cancer cells, wherein the cancer cells are contacted with a PARP inhibitor to induce DNA breaks. In another aspect, a method of treating a subject afflicted with a cancer comprising administering to the subject a therapeutically effective amount of a cancer vaccine comprising DNA repair-deficient cancer cells, wherein the cancer cells are contacted with a PARP inhibitor to induce DNA breaks, is provided. The present invention also provides a kit comprising DNA repair-deficient cancer cells modified as described herein, PARP inhibitors, immune checkpoint inhibitors, and combinations thereof, packaged in a suitable container.

### CROSS-REFERENCE TO RELATED APPLICATIONS

This application claims the benefit of U.S. Provisional Application No. 62/577,026, filed on Oct. 25, 2017; the entire contents of said application are incorporated herein in their entirety by this reference.

### STATEMENT OF RIGHTS

This invention was made with government support under grant number P01 AI056299, P50 CA101942, CA187918, and CA210057 awarded by The National Institutes of Health. The government has certain rights in the invention.

### BACKGROUND OF THE INVENTION

Poly ADP ribose polymerase (PARP) inhibitors have been recently established as an efficacious treatment for ovarian cancer with homologous recombination (HR) deficiency. Immune checkpoint CTLA-4 blockade synergizes therapeutically with PARP inhibition in BRCA1-deficient ovarian cancer (Higuchi et al. (2015) Cancer Immunol. Res. 3:1257-1268). Indeed, targeted therapy based on inhibiting DNA damage repair offers potential therapeutic approaches for the patients with tumors lacking fully competent DNA damage response functions. Among the variety of types of DNA damage, the most deleterious is DNA double-strand breaks (DSBs). DSBs can be repaired via either homologous recombination (HR) or non-homologous end joining (NHEJ). The key components of HR, the tumor suppressor genes BRCA1 and BRCA2 are frequently mutated in breast and ovarian cancers. The resulting BRCA-deficient cells rely on poly(ADP-ribose) polymerases (PARPs)-mediated DNA repair for survival, and are thus sensitive to PARP inhibition (Foulkes and Shuen (2013) J. Pathol. 230:347-349). Based on this concept of synthetic lethality between PARP inhibition and BRCA1 or BRCA2 mutation seen in tumor cells (Bryant et al. (2005) Nature 434:913-917; Farmer et al. (2005) Nature 434:917-921), therapies based on PARP inhibitors have been tested clinically and approved for the treatment of breast cancer and ovarian cancer with BRCA mutations (Lord and Ashworth (2017) Science 355:1152-1158). However, the in vivo mechanism(s) underlying the therapeutic efficacy for this novel class of drugs is unclear.

The current understanding of molecular mechanism underlying PARP inhibition for BRCA-deficient tumor is primarily described as cell autonomous synthetic lethality (O'Neil et al. (2017) Nat. Rev. Genet. 18:613-623) and increasing evidence has suggested an important interaction between tumor DNA damage and the immune system during the treatment of cancers. A recently defined endoplasmic-reticulum-associated protein, STING (stimulator of IFN genes), has been demonstrated to be a mediator for type I IFN induction by intracellular exogenous DNA in a TLR-independent manner and pro-inflammatory cytokines (Ishikawa and Barber (2008) Nature 455:674-678). STING signaling is activated by cytosolic DNAs and has been shown to play a vital role not only in protecting the cell against a variety of pathogens, but also in the antitumor immune responses in cancers (Barber (2015) Nat. Rev. Immunol. 15:760-770). STING-dependent cytosolic DNA sensing promotes radiation-induced type I IFN-dependent antitumor immunity in immunogenic tumors and mediates efficacy of radiation therapy and chemotherapy (Deng et al. (2014) Immunity 41:843-852; Parkes et al. (2016) J. Natl. Cancer Inst. 109:1). Although different anti-cancer pathways are currently under investigation, the molecular mechanisms underlying their activity and interactions are poorly understood such that a great need in the art exists to identify anti-cancer therapies based on a better understanding of the underlying mechanisms that allow for hyperproliferative cells.

### SUMMARY OF THE INVENTION

The present invention is based, at least in part, on the discovery that PARP inhibition elicits a STING-dependent antitumor immunity in HR-deficient cancer. PARP inhibitors effectively inhibit the growth of HR-deficient tumors, but not HR-proficient tumors, and such effects are further enhanced by the addition of immune checkpoint blockade (e.g., PD-1 blockade).

The present invention is also based, at least in part, on the discovery that PARP inhibition elicits an anti-tumor immune response in Brca1-deficient ovarian tumors by induction of both intratumoral and peripheral effector CD4+ and CD8+ T cells. The results presented herein further reveal that antigen-presenting cells (APCs), such as dendritic cells (DCs), can sense dsDNA fragments derived from Brca1-deficient cells upon PARP inhibition and drive a STING-dependent type I interferon signal that mediates, in part, the therapeutic efficacy of PARP inhibition in Brca1-deficient tumors. Therefore, in addition to synthetic lethality, a new mechanism of therapeutic effect of PARP inhibition in Brca1-deficient tumors that is mediated by host immune responses of the tumor-bearing host is described.

In one aspect, a cancer vaccine comprising DNA repair-deficient cancer cells, wherein the cancer cells are contacted with a PARP inhibitor to induce DNA breaks is provided.

Numerous embodiments are further provided that can be applied to any aspect of the present invention and/or combined with any other embodiment described herein. For example, in one embodiment, the cancer cells have reduced copy number, amount, and/or activity of one or more DNA damage checkpoints and/or DNA damage repair genes. In another embodiment, the one or more DNA damage checkpoints are selected from the group consisting of Brca1, Brca2, Chk1, Chk2, ATM, ATR, Cdc25C, and Nbs1. In still another embodiment, the one or more DNA damage repair genes are selected from the group consisting of non-homologous end joining (NHEJ), microhomology-mediated end joining (MMEJ), and homologous recombination pathway genes. For example, the one or more DNA damage repair genes can be selected from the group consisting of BLM, MSH2, MSH6, MLH1, PMS2, MRE11, DNA Ligase IV, TP53BP1, RAD51, RAD51L1, RAD51C, RAD51L3, DMC1, XRCC2, XRCC3, XRCC4, NBS1, RAD50, GADD45, RFC2, XRCC6, POLD2, PCNA, RPA1, RPA2, ERCC3, UNG, ERCC5, MLH1, LIG1, NBN, MSH6, POLD4, RFC5, DDB2, POLD1, FANCG, POLB, XRCC1, MPG, RFC2, ERCC1, TDG, FANCA, RFC4, RFC3, APEX2, RAD1, BRCA1, FEN1, MLH3, MGMT, RAD51, XRCC4, RECQL, ERCC8, FANCC, OGG1, MRE11A, RAD52, WRN, XPA, BLM, OGG1, MSH3, POLE2, RAD51C, LIG4, ERCC6, LIG3, RAD17, XRCC2, MUTYH, RFC1, BRCA2, RAD50, DDB1, XRCC5, PARP1, POLE3, RFC1, RAD50, XPC, MSH2, RPA3, MBD4, NTHL1, PMS2/PMS2CL, RAD51C, UNG2, APEX1, ERCC4, RAD1, RECQL5, MSH5, RECQL, RAD52, XRCC4, RAD17, MSH3, MRE11A, MSH6, and RECQL5. In yet another embodiment, the copy number, amount, and/or activity of one or more DNA damage checkpoints and/or DNA damage repair genes are reduced by contacting the cancer cells with a small molecule inhibitor, CRISPR guide RNA (gRNA), RNA interfering agent, antisense oligonucleotide, peptide or peptidomimetic inhibitor, aptamer, antibody, or intrabody. In one embodiment, the RNA interfering agent can be a small interfering RNA (siRNA), CRISPR RNA (crRNA), a CRISPR guide RNA (gRNA), a small hairpin RNA (shRNA), a microRNA (miRNA), or a piwi-interacting RNA (piRNA). In another embodiment, the antibody and/or intrabody, or antigen binding fragment thereof, specifically binds to one or more DNA damage checkpoints and/or DNA damage repair genes. In still another embodiment, the antibody and/or intrabody, or antigen binding fragment thereof, is murine, chimeric, humanized, composite, or human. In yet another embodiment, the antibody and/or intrabody, or antigen binding fragment thereof, comprises an effector domain, comprises an Fc domain, and/or is selected from the group consisting of Fv, Fav, F(ab′)2, Fab′, dsFv, scFv, sc(Fv)2, and diabodies fragments.

In another embodiment, the DNA breaks comprise double-strand DNA breaks or single-strand DNA breaks. In still another embodiment, the PARP inhibitor is selected from the group consisting of olaparib, rucaparib, niraparib, veliparib (ABT-888), talazoparib (BMN 673), iniparib (BSI-201), E7449, INO-1001, AZD2461, ME0328, TNKS49, TNKS22, JW55, PJ34, INO-1001, WIKI4, NU 1025, DR 2313, BYK 49187, BYK 204165, MK-4827, UPF 1069, A-966492, 4-HQN, EB47, MK-4827 hydrochloride, MK-4827 tosylate, and MK-4827 racemate. In yet another embodiment, the cancer cells are contacted with the PARP inhibitor alone in vitro, in vivo, and/or ex vivo, optionally wherein the cancer cells are contacted with the PARP inhibitor in combination with an immune checkpoint blockade in vitro, in vivo or ex vivo. In another embodiment, the cancer cells are contacted with the PARP inhibitor in vitro or ex vivo. In still another embodiment, the cancer cells are administered to a subject, wherein the PARP inhibitor is administered to the subject to thereby contact the cancer cells in vivo. In yet another embodiment, the PARP inhibitor is administered before, after, or concurrently with administration of the cancer cells. In another embodiment, the cancer cells are derived from a solid or hematological cancer. In still another embodiment, the cancer cells are derived from a cancer cell line. In yet another embodiment, the cancer cell line is selected from the group consisting of PP, 4T1, EMT-6, GL261, MC38, Pan02, CT26, KLN205, Lewis Lung, Madison 109, MBT-2, Colon26, CT26, A20, E.G7-OVA, B16F10, ClondmanS91, and Renca. In another embodiment, the cancer cells are ovarian cancer cells, optionally wherein the ovarian cancer cells are high grade serous ovarian cancer cells (HGSOC). In still another embodiment, the cancer cells are derived from an ovarian cancer driven by co-loss of p53 and Brca1 and overexpression of c-Myc or a breast cancer driven by co-loss of p53 and Brca1. In yet another embodiment, the cancer vaccine reduces the number of proliferating cells in the cancer and/or reduces the volume or size of a tumor comprising the cancer cells. In another embodiment, the cancer vaccine increases the amount of CD45+ immune cells infiltrating a tumor. In still another embodiment, the cancer vaccine increases the amount of intra-tumoral and peripheral IFNg⁺ TNFa⁺ CD8⁺ T cells, CD103⁺ CD8⁺ T cells, and/or IFNg⁺ TNFa⁺ CD4⁺ T cells. In yet another embodiment, the cancer vaccine decreases CD11b⁺Gr1⁺ myeloid derived suppressor cells (MDSCs) in tumor tissue and/or spleen. In another embodiment, the cancer vaccine increases intra-tumoral dendritic cells (DCs) that display an enhanced antigen presentation capacity. In still another embodiment, the cancer vaccine increases amount and/or activity of CD80, CD86, CD103, CD8a, MHC class I, and/or MHC class II on intra-tumoral DCs. In yet another embodiment, the cancer vaccine reduces intra-tumoral macrophages. In another embodiment, the cancer vaccine increases intra-tumoral M1 macrophage cells and reduces intra-tumoral M2 macrophage cells. In still another embodiment, the cancer vaccine activates STING-dependent cytosolic DNA sensing pathway in the intra-tumoral DCs and/or macrophages. In yet another embodiment, the cancer vaccine increases type I IFN in peripheral immune cells. In another embodiment, the cancer vaccine induces expression of IFN-alpha, IFN-beta, Cxcl9, Cxcl10, IL-3, IL-6, IL-7, M-CSF, TNFa, IFNg, Ccl2, Ccl5, GM-CSF, and Ccl20 in the peripheral blood and/or tumor tissues. In still another embodiment, the cancer vaccine increases dimerization and phosphorylation of STING, dimerization and nuclear translocation of IRF3, activation of IKK, phosphorylation of IkB family of inhibitors of the transcription factor NF-kB, phosphorylation of TBK1, IRF3, JAK1/2 and/or STAT1/2, and/or activates JAK/STAT pathway in the intra-tumoral DCs and/or macrophages. In yet another embodiment, the cancer cells are non-replicative, such as by irradiation. In another embodiment, the cancer vaccine is administered to a subject in combination with an immunotherapy and/or cancer therapy, optionally wherein the immunotherapy and/or cancer therapy is administered before, after, or concurrently with the cancer vaccine. In still another embodiment, the immunotherapy is cell-based. In yet another embodiment, the immunotherapy comprises a cancer vaccine and/or virus. In another embodiment, immunotherapy inhibits an immune checkpoint. In still another embodiment, the immune checkpoint is selected from the group consisting of CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRPalpha (CD47), CD48, 2B4 (CD244), B7.1, B7.2, ILT-2, ILT-4, TIGIT, HHLA2, butyrophilins, and A2aR. In yet another embodiment, the immune checkpoint is PD1, PD-L1, or CD47. In another embodiment, the cancer therapy is selected from the group consisting of radiation, a radiosensitizer, and a chemotherapy.

In another aspect, a method of treating a subject afflicted with a cancer comprising administering to the subject a therapeutically effective amount of a cancer vaccine comprising DNA repair-deficient cancer cells, wherein the cancer cells are contacted with a PARP inhibitor to induce DNA breaks, is provided.

As described above, numerous embodiments are further provided that can be applied to any aspect of the present invention and/or combined with any other embodiment described herein. For example, in one embodiment, the cancer cells have reduced copy number, amount, and/or activity of one or more DNA damage checkpoints and/or the DNA damage repair genes. In another embodiment, the one or more DNA damage checkpoints are selected from the group consisting of Brca1, Brca2, Chk1, Chk2, ATM, ATR, Cdc25C, and Nbs1. In still another embodiment, the one or more DNA damage repair genes are selected from the group consisting of non-homologous end joining (NHEJ), microhomology-mediated end joining (MMEJ), and homologous recombination pathway genes. In yet another embodiment, the one or more DNA damage repair genes are selected from the group consisting of BLM, MSH2, MSH6, MLH1, PMS2, MRE11, DNA Ligase IV, TP53BP1, RAD51, RAD51L1, RAD51C, RAD51L3, DMC1, XRCC2, XRCC3, XRCC4, NBS1, RAD50, GADD45, RFC2, XRCC6, POLD2, PCNA, RPA1, RPA2, ERCC3, UNG, ERCC5, MLH1, LIG1, NBN, MSH6, POLD4, RFC5, DDB2, POLD1, FANCG, POLB, XRCC1, MPG, RFC2, ERCC1, TDG, FANCA, RFC4, RFC3, APEX2, RAD1, BRCA1, FEN1, MLH3, MGMT, RAD51, XRCC4, RECQL, ERCC8, FANCC, OGG1, MRE11A, RAD52, WRN, XPA, BLM, OGG1, MSH3, POLE2, RAD51C, LIG4, ERCC6, LIG3, RAD17, XRCC2, MUTYH, RFC1, BRCA2, RAD50, DDB1, XRCC5, PARP1, POLE3, RFC1, RAD50, XPC, MSH2, RPA3, MBD4, NTHL1, PMS2/PMS2CL, RAD51C, UNG2, APEX1, ERCC4, RAD1, RECQL5, MSH5, RECQL, RAD52, XRCC4, RAD17, MSH3, MRE11A, MSH6, and RECQL5. In another embodiment, the copy number, amount, and/or activity of one or more DNA damage checkpoints and/or DNA damage repair genes are reduced by contacting the cancer cells with a small molecule inhibitor, CRISPR guide RNA (gRNA), RNA interfering agent, antisense oligonucleotide, peptide or peptidomimetic inhibitor, aptamer, antibody, or intrabody. In still another embodiment, the RNA interfering agent is a small interfering RNA (siRNA), CRISPR RNA (crRNA), a CRISPR guide RNA (gRNA), a small hairpin RNA (shRNA), a microRNA (miRNA), or a piwi-interacting RNA (piRNA). In yet another embodiment, the antibody and/or intrabody, or antigen binding fragment thereof, specifically binds to one or more DNA damage checkpoints and/or DNA damage repair genes. In another embodiment, the antibody and/or intrabody, or antigen binding fragment thereof, is murine, chimeric, humanized, composite, or human. In still another embodiment, the antibody and/or intrabody, or antigen binding fragment thereof, comprises an effector domain, comprises an Fc domain, and/or is selected from the group consisting of Fv, Fav, F(ab′)2, Fab′, dsFv, scFv, sc(Fv)2, and diabodies fragments.

In yet another embodiment, the DNA breaks comprise double-strand DNA breaks or single-strand DNA breaks. In another embodiment, the PARP inhibitor is selected from the group consisting of olaparib, rucaparib, niraparib, veliparib (ABT-888), talazoparib (BMN 673), iniparib (BSI-201), E7449, INO-1001, AZD2461, ME0328, TNKS49, TNKS22, JW55, PJ34, INO-1001, WIKI4, NU 1025, DR 2313, BYK 49187, BYK 204165, MK-4827, UPF 1069, A-966492, 4-HQN, EB47, MK-4827 hydrochloride, MK-4827 tosylate, and MK-4827 racemate. In still another embodiment, the cancer cells are contacted with the PARP inhibitor alone in vitro, in vivo, and/or ex vivo, optionally wherein the cancer cells are contacted with the PARP inhibitor in combination with an immune checkpoint blockade in vitro, in vivo or ex vivo. In yet another embodiment, the cancer cells are contacted with the PARP inhibitor in vitro or ex vivo. In another embodiment, the cancer cells are administered to a subject, wherein the PARP inhibitor is administered to the subject to thereby contact the cancer cells in vivo. In still another embodiment, the PARP inhibitor is administered before, after, or concurrently with administration of the cancer cells. In yet another embodiment, the cancer cells are derived from a solid or hematological cancer. In another embodiment, the cancer cells are derived from a cancer cell line. In still another embodiment, the cancer cell line is selected from the group consisting of PP, 4T1, EMT-6, GL261, MC38, Pan02, CT26, KLN205, Lewis Lung, Madison 109, MBT-2, Colon26, CT26, A20, E.G7-OVA, B16F10, ClondmanS91, and Renca. In yet another embodiment, the cancer cells are ovarian cancer cells, optionally wherein the ovarian cancer cells are high grade serous ovarian cancer cells (HGSOC). In another embodiment, the cancer cells are derived from an ovarian cancer driven by co-loss of p53 and Brca1 and overexpression of c-Myc or a breast cancer driven by co-loss of p53 and Brca1. In still another embodiment, the cancer cells are derived from a cancer that is the same type as the cancer treated with the cancer vaccine. In yet another embodiment, the cancer cells are derived from a cancer that is a different type from the cancer treated with the cancer vaccine. In another embodiment, the cancer cells are derived from the subject who is treated with the cancer vaccine. In still another embodiment, the cancer cells are derived from a different subject who is not treated with the cancer vaccine. In yet another embodiment, the cancer vaccine reduces the number of proliferating cells in the cancer and/or reduces the volume or size of a tumor comprising the cancer cells. In another embodiment, the cancer vaccine increases the amount of CD45⁺ immune cells infiltrating a tumor. In still another embodiment, the cancer vaccine increases the amount of intra-tumoral and peripheral IFNg⁺ TNFa⁺ CD8⁺ T cells, CD103⁺ CD8⁺ T cells, and/or IFNg⁺ TNFa⁺ CD4⁺ T cells. In yet another embodiment, the cancer vaccine decreases CD11b⁺Gr1⁺ myeloid derived suppressor cells (MDSCs) in the tumor tissue and/or spleen. In another embodiment, the cancer vaccine increases intra-tumoral dendritic cells (DCs) that display an enhanced antigen presenting capacity. In still another embodiment, the cancer vaccine increases amount and/or activity of CD80, CD86, CD103, CD8a, MHC class I, and/or MHC class II on intra-tumoral DCs. In yet another embodiment, the cancer vaccine reduces intra-tumoral macrophages. In another embodiment, the cancer vaccine increases intra-tumoral M1 macrophage cells and reduces M2 intra-tumoral macrophage cells. In still another embodiment, the cancer vaccine activates STING-dependent cytosolic DNA sensing pathway in the intra-tumoral DCs and/or macrophages. In yet another embodiment, the cancer vaccine increases type I IFN in the peripheral immune cells. In another embodiment, the cancer vaccine induces expression of IFN-alpha, IFN-beta, Cxcl9, Cxcl10, IL-3, IL-6, IL-7, M-CSF, TNFa, IFNg, Ccl2, Ccl5, GM-CSF, and Ccl20 in the peripheral blood and/or tumor tissues. In still another embodiment, the cancer vaccine increases dimerization and phosphorylation of STING, dimerization and nuclear translocation of IRF3, activation of IKK, phosphorylation of IkB family of inhibitors of the transcription factor NF-kB, phosphorylation of TBK1, IRF3, JAK1/2 and/or STAT1/2, and/or activation of JAK/STAT pathway in the intra-tumoral DCs and/or macrophages. In yet another embodiment, the cancer cells are non-replicative, such as by irradiation. In another embodiment, the method further comprising administering to the subject an immunotherapy and/or cancer therapy, optionally wherein the immunotherapy and/or cancer therapy is administered before, after, or concurrently with the cancer vaccine. In still another embodiment, the immunotherapy is cell-based. In yet another embodiment, the immunotherapy comprises a cancer vaccine and/or virus. In another embodiment, the immunotherapy inhibits an immune checkpoint. In still another embodiment, the immune checkpoint is selected from the group consisting of CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRPalpha (CD47), CD48, 2B4 (CD244), B7.1, B7.2, ILT-2, ILT-4, TIGIT, HHLA2, butyrophilins, and A2aR. In yet another embodiment, the immune checkpoint is PD1, PD-L1, or CD47. In another embodiment, the cancer therapy is selected from the group consisting of radiation, a radiosensitizer, and a chemotherapy.

In still another aspect, a method of assessing the efficacy of the cancer vaccine of claim 1 for treating a subject afflicted with a cancer, comprising a) detecting in a subject sample at a first point in time the number of proliferating cells in the cancer and/or the volume or size of a tumor comprising the cancer cells; b) repeating step a) during at least one subsequent point in time after administration of the cancer vaccine; and c) comparing the number of proliferating cells in the cancer and/or the volume or size of a tumor comprising the cancer cells detected in steps a) and b), wherein the absence of, or a significant decrease in number of proliferating cells in the cancer and/or the volume or size of a tumor comprising the cancer cells in the subsequent sample as compared to the number and/or the volume or size in the sample at the first point in time, indicates that the cancer vaccine treats cancer in the subject, is provided.

As described above, numerous embodiments are further provided that can be applied to any aspect of the present invention and/or combined with any other embodiment described herein. For example, in one embodiment, the subject has undergone treatment, completed treatment, and/or is in remission for the cancer between the first point in time and the subsequent point in time. In another embodiment, the first and/or at least one subsequent sample is selected from the group consisting of ex vivo and in vivo samples. In still another embodiment, the first and/or at least one subsequent sample is a portion of a single sample or pooled samples obtained from the subject. In yet another embodiment, the sample comprises cells, serum, peripheral lymphoid organs, and/or intratumoral tissue obtained from the subject. In another embodiment, the method further comprises determining responsiveness to the agent by measuring at least one criteria selected from the group consisting of clinical benefit rate, survival until mortality, pathological complete response, semi-quantitative measures of pathologic response, clinical complete remission, clinical partial remission, clinical stable disease, recurrence-free survival, metastasis free survival, disease free survival, circulating tumor cell decrease, circulating marker response, and RECIST criteria. In still another embodiment, the cancer vaccine is administered in a pharmaceutically acceptable formulation. In yet another embodiment, the step of administering occurs in vivo, ex vivo, or in vitro. In another embodiment, the cancer vaccine is administered to the subject intratumorally or subcutaneously.

In still another embodiment, the subject is an animal model of the cancer, optionally wherein the animal model is a mouse model. In yet another embodiment, the subject is a mammal, such as a mouse or a human.

### BRIEF DESCRIPTION OF THE DRAWINGS

FIG. 1A-FIG. 1I shows the results of combined PARP inhibition and PD-1 blockade in Brca1-deficient GEM model, including therapeutic efficacy of olaparib and PD-1 blockade in a Brca1-null GEM model of HGSOC. FIG. 1A shows that Brca1-deficient tumors are highly aggressive. Generation of a Brca1-null GEMM of HGSOC (Trp53−/−,Brca1−/−,Myc; termed PBM) are also shown. A representative H&E immunostaining image shows the serous carcinoma nature of the PBM tumor model. Also shown is an image of the tumor in a PBM tumor-bearing mouse. Scale bar: 25 μM. FIG. 1B shows the experimental schema (top) and representative bioluminescence-imaging analysis results of mice bearing orthotopic PBM tumor allografts (luciferized) on day 27. The tumor-bearing mice have been treated with various agents as indicated for 21 days. FIG. 1C shows a synergistic effect of PARP inhibition and PD-1 blockade on tumor growth in the Brca1-deficient GEM model. Arrow indicates treatment start date. ***, P<0.001. FIG. 1D shows the effect of the combination of PARP inhibition and PD-1 blockade on tumor growth in the Brca1-proficient PPM GEM model. Arrow indicates treatment start date. FIG. 1E shows that genetic loss of Tp53 and Brca1 and amplification/overexpression of Myc co-occur in HGSOC in clinical samples (TCGA database). FIG. 1F shows GSEA indicating an upregulated immune response and T cell activation in olaparib-treated PBM tumors. Nominal P<0.001, false discovery rate q<0.001. FIG. 1G-FIG. 1I show tumor burden of PBM tumor-bearing mice treated with indicated agents as measured by bioluminescence. FIG. 1G shows orthotopically transplanted PBM tumors in Rag1−/− or wild-type (WT) mice treated with olaparib or vehicle control (WT, n=6; Rag1−/−, n=5). FIG. 1H shows the results of PBM tumor-bearing FVB mice treated with olaparib with or without an anti-CD8 neutralizing antibody (n=8 tumors per group). FIG. 1I shows tumor burden of PBM tumor-bearing mice treated with indicated agents as measured by bioluminescence. Tumor burden was quantified by the intensity of bioluminescence signal in the regions of interest (ROI) determined at each imaging time point. Arrows indicate treatment start date. Error bars, s.e.m. *, P<0.05; **, P<0.01; ***, P<0.001.

FIG. 2A-FIG. 2N show the results of a characterization of genetically defined ovarian cancer mouse models. FIG. 2A shows the gene signature of Trp53, Brca1 and Myc for HGSOC in clinical samples (TCGA database). FIG. 2B shows the RT-qPCR analysis of the indicated mRNA expression in PBM tumors. ***, P<0.001. FIG. 2C shows the impact of olaparib and PD-1 antibody treatment on PBM tumor weight (Unpaired two-tailed t-tests. *, P<0.05, ***, P<0.001. Control, n=9; Anti-PD-1, n=10; olaparib, n=6; olaparib+Anti-PD-1, n=6). FIG. 2D shows the gene signature of Trp53, Pten and Myc for HGSOC in clinical samples (TCGA database). FIG. 2E shows a representative immunostaining which shows the serous carcinoma nature of the PPM GEM tumor model. FIG. 2F shows the RT-qPCR analysis of PD-L1 in control- and olaparib-treated tumors (control, n=7; olaparib, n=5) and in cultured PBM tumor cells treated with DMSO or olaparib (n=3). *, P<0.05; ***, P<0.001. FIG. 2G-FIG. 2N show characterization of PBM and PPM GEMMs of high-grade serous ovarian cancer (HGSOC). FIG. 2G shows RT-qPCR analysis results of expression levels of Trp53, Brca1 and c-Myc of PBM tumor cells and normal ovarian surface epithelial (OSE) cells. FIG. 2H shows analysis results of TCGA database and demonstrate concurrent loss of Pten and Trp53 and amplification of c-Myc in clinical samples of HGSOC (upper panel). Representative H&E staining and tumor images of PPM tumor (lower panel) are shown. Scale bar, 25 μm. FIG. 2I shows RT-qPCR analysis results of the expression levels of Trp53, Pten and c-Myc in PPM tumors. FIG. 2J shows the results of PBM tumor-bearing mice treated with olaparib or vehicle control for 18 days and bioluminescence measurements of tumor burden (control, n=6; olaparib, n=6). FIG. 2K shows the expression of PD-L1 of cultured PBM cells analyzed by flow cytometry following olaparib (5 μM) treatment for 24 h. FIG. 2L shows flow cytometry analysis results of PD-L1 expression of tumor cells (CD45−) harvested from PBM tumor-bearing mice. FIG. 2M shows survival curves of PBM tumor bearing mice treated with indicated agents. FIG. 2N shows the results of PPM tumor-bearing mice were treated with the indicated agents and assessed for tumor burden by bioluminescence. Quantification of the regions of interest (ROI) were determined at each imaging time point. The arrow indicates treatment start date. Error bar, s.d. (FIGS. 2G, 2I, 2L, and 2M). s.e.m. (D, H). *, P<0.05, **, P<0.01, ***, P<0.001.

FIG. 3A-FIG. 3N show that olaparib triggers intratumoral and systemic antitumor immune responses in PBM but not in PPM tumor-bearing mice. FIG. 3A and FIG. 3B show the function of tumor infiltrating CD4⁺ T cell in PBM tumors treated with PARP inhibition combined with PD-1 blockade. In FIG. 3A, FIG. 3B, and FIG. 3E-FIG. 3G, PBM tumor-bearing mice were analyzed by flow cytometry following 21 day treatment for tumor infiltrating leukocytes (CD45+). FIG. 3A and FIG. 3B show flow cytometric analysis results of tumor infiltrating leukocytes (FIG. 3A), and tumor-infiltrating Treg cells (CD4+ Foxp+) (FIG. 3B) populations in PBM tumors after treatment (22 days of treatment; Control, n=7; Anti-PD-1, n=8, Olaparib, n=6; Olaparib+Anti-PD-1, n=5). *, P<0.05. FIG. 3C and FIG. 3D show that olaparib elicits intratumoral and systemic immune responses in PBM tumor-bearing mice. FIG. 3C shows flow cytometric analysis results of intratumoral CD4+ and CD8+ T cell populations in PBM tumors treated with the indicated agents. FIG. 3D shows analysis of intratumoral CD4+ and CD8+ effector T cells (CD44highCD62Llow) in PBM tumors as determined by flow cytometry. FIG. 3E shows analysis results of intratumoral PD-1+Tim-3+ or PD-1+Lag-3+CD8+ T cells. FIG. 3F shows analysis results of effector CD8+ T cells (CD44highCD62Llow) in malignant ascites of peritoneal cavity. FIG. 3G shows analysis results of tumor infiltrating granulocytic myeloid derived suppressor cells (gMDSC, CD11b+Ly6Ghigh). FIG. 3H shows flow cytometric analysis results of PPM tumor infiltrating leukocytes. FIG. 3I shows analysis results of T cells following indicated treatment. FIG. 3J shows activation cell surface markers of dendritic cells following indicated treatment. FIG. 3K and FIG. 3L show flow cytometric analysis results of splenic immune cell populations in PBM tumor-bearing mice for CD8+ cells (FIG. 3K), and exhausted CD8+ T cells (FIG. 3L), respectively. FIG. 3M and FIG. 3N show the gating strategies for T cells (FIG. 3M), and dendritic cells (FIG. 3N), respectively. Error bar, s.d.; *, P<0.05, **, P<0.01, ***, P<0.001.

FIG. 4A-FIG. 4K show that olaparib elicits intratumoral and systemic immune responses in PBM tumor-bearing mice. FIG. 4A-FIG. 4F show that PARP inhibition combined with PD-1 blockade enhances intratumoral T cell effector function. FIG. 4A shows the results of flow cytometry analysis of intratumoral CD4⁺ and CD8⁺ T cell populations in PBM tumor (22 days of treatment; Control, n=7; Anti-PD-1, n=7, Olaparib, n=5; Olaparib+Anti-PD-1, n=5). FIG. 4B shows the intratumoral exhausted CD8⁺ T cells in PBM tumors analyzed by flow cytometry. FIG. 4C shows the flow cytometry analysis of effector cytokine (TNFα and IFNγ) production from the intratumoral CD4⁺ T cell in PBM tumors after 4 h PMA stimulation. FIG. 4D shows flow cytometry analysis results of effector cytokine production from the intratumoral CD8⁺ T cell in PBM tumors. FIG. 4E shows the results of intratumoral MDSC cells in PBM tumors after treatment. FIG. 4F shows the results of flow cytometry analysis of cell surface markers (CD80, CD86 and MHCII) expressed by tumor-infiltrating CD11c⁺ dendritic cells in PBM tumors. Unpaired two-tailed t-tests. *, P<0.05, **, P<0.01; ***, P<0.001. FIG. 4G-FIG. 4K show that olaparib elicits intratumoral and systemic immune responses in PBM tumor-bearing mice. For example, FIG. 4G and FIG. 4H show flow cytometric analysis results of effector cytokine production of intratumoral CD4+(FIG. G) and CD8+(FIG. 4H) T cell in PBM tumors treated with the indicated agents. FIG. 4I shows flow cytometric analysis results of cell surface markers (CD80, CD86, MHCII, CD103) of intratumoral CD11c+ DCs in PBM tumors. FIG. 4J and FIG. 4K show flow cytometric analysis results of blood samples from PBM tumor-bearing mice treated indicated agents. In particular, FIG. 4J shows analysis results of monocytic and granulocytic MDSCs (mMDSC and gMDSC). FIG. 4K shows analysis results of TNFα and IFNγ production CD8+ T cells. Error bars, s.d. *, P<0.05, ** P<0.01; ***, P<0.001.

FIG. 5A-FIG. 5E show that systemic immunity is modulated by PARP inhibition and PD-1 blockade. FIG. 5A shows TNFα and IFNγ production from peripheral blood CD8⁺ T cells after 4 h PMA stimulation. FIG. 5B-FIG. 5D show flow cytometric analysis results of splenic CD8+ cells (FIG. 5B), CD8⁺ exhausted T cells (FIG. 5C), and MDSC cells (FIG. 5D) in PBM tumors after treatment (22 days of treatment; Control, n=6; Anti-PD-1, n=8, Olaparib, n=6; Olaparib+Anti-PD-1, n=6). Unpaired two-tailed t-tests. *, P<0.05, **, P<0.01; ***, P<0.001. FIG. 5E shows cytokine array analysis results of plasma in control- or olaparib-treated PBM tumor-bearing mice on day 2 of treatment. (Control, n=7; Olaparib, n=5). *, P<0.05, **, P<0.01.

FIG. 6 shows the results of modulation of Treg populations by PARP inhibition in combination with PD-1 blockade in PBM tumors. Splenic Treg cells were analyzed by flow cytometry (22 days of treatment; Control, n=7; Anti-PD-1, n=8, Olaparib, n=6; Olaparib+Anti-PD-1, n=5).

FIG. 7A-FIG. 7AA show that PARP inhibition activates the STING pathway in intratumoral DCs in PBM tumors. FIG. 7A shows the measurement of type I interferon IFN-β and chemokines, CXCL9 and CXCL10, by cytokine array with plasma from control- or olaparib-treated PBM tumors (day 2) (IFN-β: control, n=8; Olaparib, n=6; CXCL9 and CXCL10: control, n=9; Olaparib, n=7). FIG. 7B shows RT-qPCR analysis results of IFN-β and CXCL10 in control- or olaparib-treated PBM tumors (left) (control, n=7; Olaparib, n=5), and flow cytometric analysis results of phosphorylated TBK1 and IRF-3 in dendritic cells from PBM tumors with or without olaparib treatment (right) (Control, n=3; Olaparib, n=3). FIG. 7C shows RT-qPCR analysis results of IFN-β and CXCL10 in DCs isolated from control- or olaparib-treated PPM tumors (left) (PPM, control, n=7; Olaparib, n=6), and flow cytometric analysis results of phosphorylated TBK1 and IRF-3 in tumor-infiltrating dendritic cells in control- or olaparib-treated PPM tumors (right) (Control, n=6; Olaparib, n=6). Error bar, s.d. *, P<0.05; **, P<0.01. FIG. 7D shows the results of analysis of secreted IFN-β in the media of BMDC cells co-cultured with olaparib-treated PBM tumor cells by ELISA. BX795, inhibitor of TBK1 (left), and the RT-qPCR analysis of CXCL10 expression by BMDCs cultured with PBM tumor cells with or without BX795 (right) are shown. The P value shows the statistical significance between samples that had DCs co-cultured with either control- or olaparib treated tumor cells. FIG. 7E shows the results of analysis of secreted IFN-β in the media and levels of IFN-β and CXCL10 mRNA in BMDCs cultured with either control- or olaparib-treated PPM tumor cells. IFN-1 in the co-culture media and IFN-β and CXCL10 mRNA in BMDCs were analyzed by ELISA and RT-qPCR, respectively. Unpaired two-tailed t-tests. *, P<0.05, **, P<0.01; ***, P<0.001. FIG. 7F shows flow cytometric analysis results of TBK1 phosphorylated-CD11c dendritic cells in the same co-culture system of FIG. 7D (Control, n=3; Olaparib, n=3). **, P<0.01, ***, P<0.001. FIG. 7G shows that BX795, a STING agonist, blocked the anti-tumor activity of olaparib on PBM tumors. **, P<0.01. FIG. 7H shows that anti-IFNAR1 antibody attenuated the anti-tumor effects of olaprib on the Brca1-deficient PBM GEM model. **, P<0.01. The arrow indicates the treatment start date. FIG. 7I-FIG. 7P show that olaparib treated Brca1-deficient tumor cells trigger the STING-pathway activation in DCs in a co-culture system. FIG. 7I shows the staining of cytosolic double strand DNA (dsDNA) in PBM tumor cells treated with DMSO or olaparib (2.5 μM, 24 h); scale bar, 25 m; quantification data shown in bar. FIG. 7J illustrates a co-culture system with BMDCs and olaparib treated cells. FIG. 7K shows flow cytometric analysis results of STING pathway activation (p-TBK1+p-IRF3+) in BMDCs co-cultured with olaparib-treated PBM tumor cells, in the presence or absence of a STING inhibitor BX795. FIG. 7L shows RT-qPCR analysis results of IFN-β and CXCL10 expression in BMDCs collected from BMDC/PBM co-culture. FIG. 7M and FIG. 7N show the results of human DCs co-cultured with olaparib-treated human ovarian cancer cell lines, UWB1.289 and UWB1.289+BRCA1. For example, FIG. 7M shows flow cytometric analysis results of phosphorylated TBK1 and IRF3, and FIG. 7N shows RT qPCR analysis results of IFN-β expression in human DCs from co-culture. FIG. 7O and FIG. 7P show the results of wild type (WT) and STING−/− BMDCs co-cultured with WT- or Brca1-null ID8 tumor cells pre-treated with DMSO or olaparib. For example, FIG. 7O shows flow cytometric analysis results of p-TBK1+p-IRF3+, and FIG. 7P shows RT-qPCR analysis results of IFN-β expression level in DCs from co-culture with ID8 cells (Control, n=3; Olaparib, n=3). Error bars, s.d. *, P<0.05, **, P<0.01; ***, P<0.001. FIG. 7Q-FIG. 7AA show activation of the STING pathway in dendritic cells co-cultured with olaparib-treated Brca1-deficient tumor cells. FIG. 7Q shows representative staining of cytosolic double strand DNA (dsDNA) by PicoGreen and quantification in PPM tumor cells treated with DMSO or 2.5 μM olaparib for 24 h, scale bar, 25 μm. FIG. 7R shows representative DAPI staining of primary nucleus and micronucleus (indicated by arrows). The percentage of cells with micronucleus was calculated (left). FIG. 7S shows Western blot analysis results of cGAS-STING and its signaling molecules in olaparib-treated PBM and PPM tumors cells. Bone marrow-derived dendritic cells (BMDCs) treated with DMXAA, a murine STING agonist, served as a positive control. FIG. 7T shows analysis results of IFN-β level in BMDCs culture media and results of cells analyzed by ELISA (left) and RT-qPCR (right), respectively. To modulate STING signaling pathway, DMXAA (5 μg/ml, 2 h) and a STING inhibitor, BX795 (2 μM, 2 h), were used. FIG. 7U shows flow cytometric analysis results of STING pathway activation (indicated by phosphorylated TBK1 and IRF3) in BMDCs from BMDC/PBM co-culture. Prior to co-culture with BMDCs, PBM cells pre-treated with olaparib (2.5 μM) in the presence or absence of an apoptosis inhibitor, zVAD (10 μM), for 24 h. FIG. 7V shows low cytometric analysis results of phosphorylated TBK1 and IRF3 (left) and RT-qPCR of IFN-β (right) in wild type (WT) and STING−/− BMDCs stimulated with DMXAA (5 μg/ml) for 2 hours. FIG. 7W shows IC50 values for PARP inhibitors and other cytotoxic agents in BRCA1-proficient and BRCA1-deficient tumor cells. FIG. 7X and FIG. 7Y show flow cytometric analysis results of phosphorylated TBK1 and IRF3 in BMDCs co-cultured with WT or Brca1-null ID8 cells with the indicated treatments. FIG. 7Z and FIG. 7AA show the gating strategies of phosphorylated TBK1 and IRF3 in CD11c+dendritic cells in murine BMDCs (FIG. 7Z) and human DCs (FIG. 7AA). Error bar, s.d.; *, P<0.05, **, P<0.01, ***, P<0.001.

FIG. 8A-FIG. 8C show the activation of the STING pathway mediated by macrophages upon PARP inhibition. FIG. 8A shows that phosphorylation of TBK1 and IRF-3 in macrophages was detected by flow cytometry in control- and olaparib-treated tumors (Control, n=5; Olaparib, n=5). **, P<0.01. FIG. 8B shows results of ELISA analysis of IFNβ level in the media of co-cultured bone marrow-derived macrophage cells and olaparib-treated PBM tumor cells. FIG. 8C shows the detection of IFN-β level by ELISA (left) or RT-qPCR (right) in the media of BMDCs that were stimulated with DMXAA (5 μg/ml) in the presence or absence of BX795 (2 μM).

FIG. 9A-FIG. 9B show that olaparib suppressed the growth of PP tumors with intratumoral injection of Brca1-deficient cells. FIG. 9A shows results of 1×10⁶ PBM or PP cells in 100 μl DMEM administered to established PP tumors via intratumoral injection (IT) 3 times (1^{st} time, day 1; 2^{nd} time, day 5; 3^{rd}, day 9). Mice received 50 mg/kg/day olaparib via IP from day 1. Data are shown as mean±SEM; Unpaired Student's t test. FIG. 9B shows that the established PP tumors received two intratumoral injections of 500 ug of DMXAA, a STING agonist, (DMXAA injected) or vehicle control over a one week period. Results are shown as mean±SEM. N=6 (control).

FIG. 10 shows flow cytometry analysis results of tumors harvested from indicated groups for CD45+ cells, dendritic cells (CD45+CD11c+MHC II+), and macrophages (CD45+CD11b+F4/80+).

FIG. 11 shows the results of tumors from indicated groups harvested and analyzed by flow cytometry. The percentage of M1 (MHC II high; CD206 low) and M2 (MHC II low; CD206 high) macrophages, respectively, over the total tumor-associated macrophages, are shown. CD206, mannose receptor. MHC II, major histocompatibility complex class II.

FIG. 12 shows the results of flow cytometry analysis of tumors harvested from the indicated groups for p-IRF3 and p-TBK1 double-positive dendritic cells and macrophages, respectively. The left panel shows representative FACS blots. The right panel shows the percentage of p-IRF3+p-TBK1+ among dendritic cells and macrophages, respectively. Data are shown as mean±SEM.

FIG. 13A-FIG. 13H show that the activation of the STING pathway is required for olaparib triggered antitumor immunity in Brca1-deficient tumors. FIG. 13A shows flow cytometric analysis results of p-TBK1+p-IRF3+ DCs and macrophages from PBM tumors. FIG. 13B shows the expression of IFN-β and CXCL10 in PBM tumor tissues harvested from PBM tumor-bearing mice treated with vehicle control or olaparib by RT-qPCR analysis (Control, n=7; Olaparib, n=5). FIG. 13C shows tumor growth results in mice bearing orthotopic allografts of luciferized PBM tumors treated with olaparib with or without BX795 (Control, n=8; Olaparib, n=9; BX795, n=7; olaparib+BX795, n=7). FIG. 13D shows tumor growth results in mice bearing orthotopic allografts of luciferized PBM tumors treated with olaparib with or without anti-IFNAR1 (Control, n=10; Olaparib, n=9; Anti-IFNAR1, n=7; Olaparib+Anti-IFNAR1, n=8). FIG. 13E shows measurements of tumor weights. Brca1-null ID8 cells were subcutaneously injected into WT or STING−/− mice and treated with olaparib or vehicle control. FIG. 13F shows flow cytometric analysis results of p-TBK1 in intratumoral DCs of Brca1 null ID8 tumor from FIG. 13E. The arrows indicate treatment start date. Error bars, s.d. (FIG. 13A and FIG. 13B), s.e.m. (FIG. 13C to FIG. 13F). *, P<0.05, ** P<0.01, ***, P<0.001. #, compared to the control group; ##, P<0.01; ###, P<0.001. FIG. 13G and FIG. 13H show that STING pathway activation is required for the therapeutic efficacy of PARP inhibitors in Brca1-deficient tumors. FIG. 13G shows the gating strategies of phosphorylated TBK1 and IRF3 of CD11c+ dendritic cells in PBM and PPM tumors. FIG. 13H shows cytokine array results of sera collected from PBM tumor-bearing mice treated with vehicle or olaparib for 2 days (Control, n=9; Olaparib, n=7).

For any figure showing a bar histogram, curve, or other data associated with a legend, the bars, curve, or other data presented from left to right for each indication correspond directly and in order to the boxes from top to bottom of the legend.

### DETAILED DESCRIPTION OF THE INVENTION

It has been determined herein that PARP inhibitors, such as olaparib, effectively inhibit the growth of HR-deficient tumors, but not HR-proficient tumors, as a single-agent, and that the effect is further enhanced by also inhibiting immune checkpoints (e.g., PD-1 blockade). For example, a marked increase occurs in both intra-tumoral and peripheral IFNg+TNFa+CD4+ T cells and IFNg+TNFa+CD8+ T cells in HR-deficient tumor-bearing mice following olaparib treatment alone. This increased activation of T cells is associated with increased recruitment of dendritic cells (DCs) displaying potent antigen presentation capacity in the tumor microenvironment and accompanied by significantly reduced myeloid derived suppressor cells (MDSCs) in the tumor tissue and the spleen. Mechanistically, this coordinated robust local and systemic anti-tumor immunity involving both adaptive and innate immune responses upon PARP inhibition in this Brca1-deficient tumor model is through the activation of the STING (stimulator of IFN genes) pathway in antigen presenting cells mediated by the sensing of tumor-derived DNA. These determinations demonstrate a STING-dependent mechanism underlying the therapeutic efficacy of PARP inhibitors for the treatment of HR-deficient tumors.

Accordingly, based on the findings that PARP inhibition elicits a STING-dependent antitumor immunity in HR-deficient cancer, the present invention relates, in part, to compositions and methods for treating cancer using a cancer vaccine that triggers a STING-dependent antitumor immunity.

### I. Definitions

The articles “a” and “an” are used herein to refer to one or to more than one (i.e. to at least one) of the grammatical object of the article. By way of example, “an element” means one element or more than one element.

The term “administering” is intended to include routes of administration which allow an agent to perform its intended function. Examples of routes of administration for treatment of a body which can be used include injection (subcutaneous, intravenous, parenteral, intraperitoneal, intrathecal, etc.), oral, inhalation, and transdermal routes. The injection can be bolus injections or can be continuous infusion. Depending on the route of administration, the agent can be coated with or disposed in a selected material to protect it from natural conditions which may detrimentally affect its ability to perform its intended function. The agent may be administered alone, or in conjunction with a pharmaceutically acceptable carrier. The agent also may be administered as a prodrug, which is converted to its active form in vivo.

The term “altered amount” or “altered level” refers to increased or decreased copy number (e.g., germline and/or somatic) of a biomarker nucleic acid, e.g., increased or decreased expression level in a cancer sample, as compared to the expression level or copy number of the biomarker nucleic acid in a control sample. The term “altered amount” of a biomarker also includes an increased or decreased protein level of a biomarker protein in a sample, e.g., a cancer sample, as compared to the corresponding protein level in a normal, control sample. Furthermore, an altered amount of a biomarker protein may be determined by detecting posttranslational modification such as methylation status of the marker, which may affect the expression or activity of the biomarker protein.

The amount of a biomarker in a subject is “significantly” higher or lower than the normal amount of the biomarker, if the amount of the biomarker is greater or less, respectively, than the normal level by an amount greater than the standard error of the assay employed to assess amount, and preferably at least 20%, 30%, 40%, 50%, 60%, 70%, 80%, 90%, 100%, 150%, 200%, 300%, 350%, 400%, 500%, 600%, 700%, 800%, 900%, 1000% or than that amount. Alternately, the amount of the biomarker in the subject can be considered “significantly” higher or lower than the normal amount if the amount is at least about two, and preferably at least about three, four, or five times, higher or lower, respectively, than the normal amount of the biomarker. Such “significance” can also be applied to any other measured parameter described herein, such as for expression, inhibition, cytotoxicity, cell growth, and the like.

The term “altered level of expression” of a biomarker refers to an expression level or copy number of the biomarker in a test sample, e.g., a sample derived from a patient suffering from cancer, that is greater or less than the standard error of the assay employed to assess expression or copy number, and is preferably at least twice, and more preferably three, four, five or ten or more times the expression level or copy number of the biomarker in a control sample (e.g., sample from a healthy subjects not having the associated disease) and preferably, the average expression level or copy number of the biomarker in several control samples. The altered level of expression is greater or less than the standard error of the assay employed to assess expression or copy number, and is preferably at least 20%, 30%, 40%, 50%, 60%, 70%, 80%, 90%, 100%, 150%, 200%, 300%, 350%, 400%, 500%, 600%, 700%, 800%, 900%, 1000% or more times the expression level or copy number of the biomarker in a control sample (e.g., sample from a healthy subjects not having the associated disease) and preferably, the average expression level or copy number of the biomarker in several control samples. In some embodiments, the level of the biomarker refers to the level of the biomarker itself, the level of a modified biomarker (e.g., phosphorylated biomarker), or to the level of a biomarker relative to another measured variable, such as a control (e.g., phosphorylated biomarker relative to an unphosphorylated biomarker).

The term “altered activity” of a biomarker refers to an activity of the biomarker which is increased or decreased in a disease state, e.g., in a cancer sample, as compared to the activity of the biomarker in a normal, control sample. Altered activity of the biomarker may be the result of, for example, altered expression of the biomarker, altered protein level of the biomarker, altered structure of the biomarker, or, e.g., an altered interaction with other proteins involved in the same or different pathway as the biomarker or altered interaction with transcriptional activators or inhibitors.

The term “altered structure” of a biomarker refers to the presence of mutations or allelic variants within a biomarker nucleic acid or protein, e.g., mutations which affect expression or activity of the biomarker nucleic acid or protein, as compared to the normal or wild-type gene or protein. For example, mutations include, but are not limited to substitutions, deletions, or addition mutations. Mutations may be present in the coding or non-coding region of the biomarker nucleic acid.

Unless otherwise specified here within, the terms “antibody” and “antibodies” broadly encompass naturally-occurring forms of antibodies (e.g. IgG, IgA, IgM, IgE) and recombinant antibodies, such as single-chain antibodies, chimeric and humanized antibodies and multi-specific antibodies, as well as fragments and derivatives of all of the foregoing, which fragments and derivatives have at least an antigenic binding site. Antibody derivatives may comprise a protein or chemical moiety conjugated to an antibody.

In addition, intrabodies are well-known antigen-binding molecules having the characteristic of antibodies, but that are capable of being expressed within cells in order to bind and/or inhibit intracellular targets of interest (Chen et al. (1994) Human Gene Ther. 5:595-601). Methods are well-known in the art for adapting antibodies to target (e.g., inhibit) intracellular moieties, such as the use of single-chain antibodies (scFvs), modification of immunoglobulin VL domains for hyperstability, modification of antibodies to resist the reducing intracellular environment, generating fusion proteins that increase intracellular stability and/or modulate intracellular localization, and the like. Intracellular antibodies can also be introduced and expressed in one or more cells, tissues or organs of a multicellular organism, for example for prophylactic and/or therapeutic purposes (e.g., as a gene therapy) (see, at least PCT Publs. WO 08/020079, WO 94/02610, WO 95/22618, and WO 03/014960; U.S. Pat. No. 7,004,940; Cattaneo and Biocca (1997) Intracellular Antibodies: Development and Applications (Landes and Springer-Verlag publs.); Kontermann (2004) Methods 34:163-170; Cohen et al. (1998) Oncogene 17:2445-2456; Auf der Maur et al. (2001) FEBS Lett. 508:407-412; Shaki-Loewenstein et al. (2005) J. Immunol. Meth. 303:19-39).

The term “antibody” as used herein also includes an “antigen-binding portion” of an antibody (or simply “antibody portion”). The term “antigen-binding portion”, as used herein, refers to one or more fragments of an antibody that retain the ability to specifically bind to an antigen (e.g., a biomarker polypeptide or fragment thereof). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent polypeptides (known as single chain Fv (scFv); see e.g., Bird et al. (1988) Science 242:423-426; and Huston et al. (1988) Proc. Natl. Acad. Sci. USA 85:5879-5883; and Osbourn et al. 1998, Nature Biotechnology 16: 778). Such single chain antibodies are also intended to be encompassed within the term “antigen-binding portion” of an antibody. Any VH and VL sequences of specific scFv can be linked to human immunoglobulin constant region cDNA or genomic sequences, in order to generate expression vectors encoding complete IgG polypeptides or other isotypes. VH and VL can also be used in the generation of Fab, Fv or other fragments of immunoglobulins using either protein chemistry or recombinant DNA technology. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger et al. (1993) Proc. Natl. Acad. Sci. U.S.A. 90:6444-6448; Poljak et al. (1994) Structure 2:1121-1123).

Still further, an antibody or antigen-binding portion thereof may be part of larger immunoadhesion polypeptides, formed by covalent or noncovalent association of the antibody or antibody portion with one or more other proteins or peptides. Examples of such immunoadhesion polypeptides include use of the streptavidin core region to make a tetrameric scFv polypeptide (Kipriyanov et al. (1995) Human Antibodies and Hybridomas 6:93-101) and use of a cysteine residue, biomarker peptide and a C-terminal polyhistidine tag to make bivalent and biotinylated scFv polypeptides (Kipriyanov et al. (1994) Mol. Immunol. 31:1047-1058). Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion polypeptides can be obtained using standard recombinant DNA techniques, as described herein.

Antibodies may be polyclonal or monoclonal; xenogeneic, allogeneic, or syngeneic; or modified forms thereof (e.g. humanized, chimeric, etc.). Antibodies may also be fully human. Preferably, antibodies of the invention bind specifically or substantially specifically to a biomarker polypeptide or fragment thereof. The terms “monoclonal antibodies” and “monoclonal antibody composition”, as used herein, refer to a population of antibody polypeptides that contain only one species of an antigen binding site capable of immunoreacting with a particular epitope of an antigen, whereas the term “polyclonal antibodies” and “polyclonal antibody composition” refer to a population of antibody polypeptides that contain multiple species of antigen binding sites capable of interacting with a particular antigen. A monoclonal antibody composition typically displays a single binding affinity for a particular antigen with which it immunoreacts.

Antibodies may also be “humanized,” which is intended to include antibodies made by a non-human cell having variable and constant regions which have been altered to more closely resemble antibodies that would be made by a human cell. For example, by altering the non-human antibody amino acid sequence to incorporate amino acids found in human germline immunoglobulin sequences. The humanized antibodies of the invention may include amino acid residues not encoded by human germline immunoglobulin sequences (e.g., mutations introduced by random or site-specific mutagenesis in vitro or by somatic mutation in vivo), for example in the CDRs. The term “humanized antibody”, as used herein, also includes antibodies in which CDR sequences derived from the germline of another mammalian species, have been grafted onto human framework sequences.

The term “biomarker” refers to a measurable entity of the present invention that has been determined to be predictive of cancer therapy effects. Biomarkers can include, without limitation, nucleic acids (e.g., genomic nucleic acids and/or transcribed nucleic acids) and proteins. Many biomarkers are also useful as therapeutic targets.

A “blocking” antibody or an antibody “antagonist” is one which inhibits or reduces at least one biological activity of the antigen(s) it binds. In certain embodiments, the blocking antibodies or antagonist antibodies or fragments thereof described herein substantially or completely inhibit a given biological activity of the antigen(s).

The term “body fluid” refers to fluids that are excreted or secreted from the body as well as fluids that are normally not (e.g. amniotic fluid, aqueous humor, bile, blood and blood plasma, cerebrospinal fluid, cerumen and earwax, cowper's fluid or pre-ejaculatory fluid, chyle, chyme, stool, female ejaculate, interstitial fluid, intracellular fluid, lymph, menses, breast milk, mucus, pleural fluid, pus, saliva, sebum, semen, serum, sweat, synovial fluid, tears, urine, vaginal lubrication, vitreous humor, vomit).

The terms “cancer” or “tumor” or “hyperproliferative” refer to the presence of cells possessing characteristics typical of cancer-causing cells, such as uncontrolled proliferation, immortality, metastatic potential, rapid growth and proliferation rate, and certain characteristic morphological features.

Cancer cells are often in the form of a tumor, but such cells may exist alone within an animal, or may be a non-tumorigenic cancer cell, such as a leukemia cell. As used herein, the term “cancer” includes premalignant as well as malignant cancers. Cancers include, but are not limited to, B cell cancer, e.g., multiple myeloma, Waldenstrom's macroglobulinemia, the heavy chain diseases, such as, for example, alpha chain disease, gamma chain disease, and mu chain disease, benign monoclonal gammopathy, and immunocytic amyloidosis, melanomas, breast cancer, lung cancer, bronchus cancer, colorectal cancer, prostate cancer, pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematologic tissues, and the like. Other non-limiting examples of types of cancers applicable to the methods encompassed by the present invention include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, cancers are epithelial in nature and include but are not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers may be characterized in various other ways including, but not limited to, serous, endometrioid, mucinous, clear cell, Brenner, or undifferentiated.

The term “coding region” refers to regions of a nucleotide sequence comprising codons which are translated into amino acid residues, whereas the term “noncoding region” refers to regions of a nucleotide sequence that are not translated into amino acids (e.g., 5′ and 3′ untranslated regions).

The term “complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

The terms “conjoint therapy” and “combination therapy,” as used herein, refer to the administration of two or more therapeutic substances. The different agents comprising the combination therapy may be administered concomitant with, prior to, or following the administration of one or more therapeutic agents.

The term “control” refers to any reference standard suitable to provide a comparison to the expression products in the test sample. In one embodiment, the control comprises obtaining a “control sample” from which expression product levels are detected and compared to the expression product levels from the test sample. Such a control sample may comprise any suitable sample, including but not limited to a sample from a control cancer patient (can be stored sample or previous sample measurement) with a known outcome; normal tissue or cells isolated from a subject, such as a normal patient or the cancer patient, cultured primary cells/tissues isolated from a subject such as a normal subject or the cancer patient, adjacent normal cells/tissues obtained from the same organ or body location of the cancer patient, a tissue or cell sample isolated from a normal subject, or a primary cells/tissues obtained from a depository. In another preferred embodiment, the control may comprise a reference standard expression product level from any suitable source, including but not limited to housekeeping genes, an expression product level range from normal tissue (or other previously analyzed control sample), a previously determined expression product level range within a test sample from a group of patients, or a set of patients with a certain outcome (for example, survival for one, two, three, four years, etc.) or receiving a certain treatment (for example, standard of care cancer therapy). It will be understood by those of skill in the art that such control samples and reference standard expression product levels can be used in combination as controls in the methods of the present invention. In one embodiment, the control may comprise normal or non-cancerous cell/tissue sample. In another preferred embodiment, the control may comprise an expression level for a set of patients, such as a set of cancer patients, or for a set of cancer patients receiving a certain treatment, or for a set of patients with one outcome versus another outcome. In the former case, the specific expression product level of each patient can be assigned to a percentile level of expression, or expressed as either higher or lower than the mean or average of the reference standard expression level. In another preferred embodiment, the control may comprise normal cells, cells from patients treated with combination chemotherapy, and cells from patients having benign cancer. In another embodiment, the control may also comprise a measured value for example, average level of expression of a particular gene in a population compared to the level of expression of a housekeeping gene in the same population. Such a population may comprise normal subjects, cancer patients who have not undergone any treatment (i.e., treatment naive), cancer patients undergoing standard of care therapy, or patients having benign cancer. In another preferred embodiment, the control comprises a ratio transformation of expression product levels, including but not limited to determining a ratio of expression product levels of two genes in the test sample and comparing it to any suitable ratio of the same two genes in a reference standard; determining expression product levels of the two or more genes in the test sample and determining a difference in expression product levels in any suitable control; and determining expression product levels of the two or more genes in the test sample, normalizing their expression to expression of housekeeping genes in the test sample, and comparing to any suitable control. In particularly preferred embodiments, the control comprises a control sample which is of the same lineage and/or type as the test sample. In another embodiment, the control may comprise expression product levels grouped as percentiles within or based on a set of patient samples, such as all patients with cancer. In one embodiment a control expression product level is established wherein higher or lower levels of expression product relative to, for instance, a particular percentile, are used as the basis for predicting outcome. In another preferred embodiment, a control expression product level is established using expression product levels from cancer control patients with a known outcome, and the expression product levels from the test sample are compared to the control expression product level as the basis for predicting outcome. As demonstrated by the data below, the methods of the invention are not limited to use of a specific cut-point in comparing the level of expression product in the test sample to the control.

The “copy number” of a biomarker nucleic acid refers to the number of DNA sequences in a cell (e.g., germline and/or somatic) encoding a particular gene product. Generally, for a given gene, a mammal has two copies of each gene. The copy number can be increased, however, by gene amplification or duplication, or reduced by deletion. For example, germline copy number changes include changes at one or more genomic loci, wherein said one or more genomic loci are not accounted for by the number of copies in the normal complement of germline copies in a control (e.g., the normal copy number in germline DNA for the same species as that from which the specific germline DNA and corresponding copy number were determined). Somatic copy number changes include changes at one or more genomic loci, wherein said one or more genomic loci are not accounted for by the number of copies in germline DNA of a control (e.g., copy number in germline DNA for the same subject as that from which the somatic DNA and corresponding copy number were determined).

The term “immune cell” refers to cells that play a role in the immune response. Immune cells are of hematopoietic origin, and include lymphocytes, such as B cells and T cells; natural killer cells; myeloid cells, such as monocytes, macrophages, eosinophils, mast cells, basophils, and granulocytes.

Macrophages (and their precursors, monocytes) are the ‘big eaters’ of the immune system. These cells reside in every tissue of the body, albeit in different guises, such as microglia, Kupffer cells and osteoclasts, where they engulf apoptotic cells and pathogens and produce immune effector molecules. Upon tissue damage or infection, monocytes are rapidly recruited to the tissue, where they differentiate into tissue macrophages. Macrophages are remarkably plastic and can change their functional phenotype depending on the environmental cues they receive. Through their ability to clear pathogens and instruct other immune cells, these cells have a central role in protecting the host but also contribute to the pathogenesis of inflammatory and degenerative diseases. Macrophages that encourage inflammation are called M1 macrophages, whereas those that decrease inflammation and encourage tissue repair are called M2 macrophages. M1 macrophages are activated by LPS and IFN-gamma, and secrete high levels of IL-12 and low levels of IL-10. M2 is the phenotype of resident tissue macrophages, and can be further elevated by IL-4. M2 macrophages produce high levels of IL-10, TGF-beta and low levels of IL-12. Tumor-associated macrophages are mainly of the M2 phenotype, and seem to actively promote tumor growth.

Myeloid derived suppressor cells (MDSCs) are an intrinsic part of the myeloid cell lineage and are a heterogeneous population comprised of myeloid cell progenitors and precursors of granulocytes, macrophages and dendritic cells. MDSCs are defined by their myeloid origin, immature state and ability to potently suppress T cell responses. They regulate immune responses and tissue repair in healthy individuals and the population rapidly expands during inflammation, infection and cancer. MDSC are one of the major components of the tumor microenvironment. The main feature of these cells is their potent immune suppressive activity. MDSC are generated in the bone marrow and, in tumor-bearing hosts, migrate to peripheral lymphoid organs and the tumor to contribute to the formation of the tumor microenvironment. This process is controlled by a set of defined chemokines, many of which are upregulated in cancer. Hypoxia appears to have a critical role in the regulation of MDSC differentiation and function in tumors. Therapeutic strategies are now being developed to target MDSCs to promote antitumour immune responses or to inhibit immune responses in the setting of autoimmune disease or transplant rejection.

Dendritic cells (DCs) are professional antigen-presenting cells located in the skin, mucosa and lymphoid tissues. Their main function is to process antigens and present them to T cells to promote immunity to foreign antigens and tolerance to self antigens. They also secrete cytokines to regulate immune responses.

Conventional T cells, also known as Tconv or Teffs, have effector functions (e.g., cytokine secretion, cytotoxic activity, anti-self-recognization, and the like) to increase immune responses by virtue of their expression of one or more T cell receptors. Tcons or Teffs are generally defined as any T cell population that is not a Treg and include, for example, naïve T cells, activated T cells, memory T cells, resting Tcons, or Tcons that have differentiated toward, for example, the Th1 or Th2 lineages. In some embodiments, Teffs are a subset of non-Treg T cells. In some embodiments, Teffs are CD4+ Teffs or CD8+ Teffs, such as CD4+ helper T lymphocytes (e.g., Th0, Th1, Tfh, or Th17) and CD8+ cytotoxic T lymphocytes. As described further herein, cytotoxic T cells are CD8+T lymphocytes. “Naïve Tcons” are CD4⁺ T cells that have differentiated in bone marrow, and successfully underwent a positive and negative processes of central selection in a thymus, but have not yet been activated by exposure to an antigen. Naïve Tcons are commonly characterized by surface expression of L-selectin (CD62L), absence of activation markers such as CD25, CD44 or CD69, and absence of memory markers such as CD45RO. Naïve Tcons are therefore believed to be quiescent and non-dividing, requiring interleukin-7 (IL-7) and interleukin-15 (IL-15) for homeostatic survival (see, at least WO 2010/101870). The presence and activity of such cells are undesired in the context of suppressing immune responses. Unlike Tregs, Tcons are not anergic and can proliferate in response to antigen-based T cell receptor activation (Lechler et al. (2001) Philos. Trans. R. Soc. Lond. Biol. Sci. 356:625-637). In tumors, exhausted cells can present hallmarks of anergy.

The term “immunotherapy” or “immunotherapies” refer to any treatment that uses certain parts of a subject's immune system to fight diseases such as cancer. The subject's own immune system is stimulated (or suppressed), with or without administration of one or more agent for that purpose. Immunotherapies that are designed to elicit or amplify an immune response are referred to as “activation immunotherapies.” Immunotherapies that are designed to reduce or suppress an immune response are referred to as “suppression immunotherapies.” Any agent believed to have an immune system effect on the genetically modified transplanted cancer cells can be assayed to determine whether the agent is an immunotherapy and the effect that a given genetic modification has on the modulation of immune response. In some embodiments, the immunotherapy is cancer cell-specific. In some embodiments, immunotherapy can be “untargeted,” which refers to administration of agents that do not selectively interact with immune system cells, yet modulates immune system function. Representative examples of untargeted therapies include, without limitation, chemotherapy, gene therapy, and radiation therapy.

Immunotherapy is one form of targeted therapy that may comprise, for example, the use of cancer vaccines and/or sensitized antigen presenting cells. For example, an oncolytic virus is a virus that is able to infect and lyse cancer cells, while leaving normal cells unharmed, making them potentially useful in cancer therapy. Replication of oncolytic viruses both facilitates tumor cell destruction and also produces dose amplification at the tumor site. They may also act as vectors for anticancer genes, allowing them to be specifically delivered to the tumor site. The immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). For example, anti-VEGF and mTOR inhibitors are known to be effective in treating renal cell carcinoma. Immunotherapy can also focus on using the cytotoxic lymphocyte-recognized epitopes of cancer cell lines. Alternatively, antisense polynucleotides, ribozymes, RNA interference molecules, triple helix polynucleotides and the like, can be used to selectively modulate biomolecules that are linked to the initiation, progression, and/or pathology of a tumor or cancer.

Immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). Immunotherapy can also focus on using the cytotoxic lymphocyte-recognized epitopes of cancer cell lines. Alternatively, antisense polynucleotides, ribozymes, RNA interference molecules, triple helix polynucleotides and the like, can be used to selectively modulate biomolecules that are linked to the initiation, progression, and/or pathology of a tumor or cancer.

In some embodiments, immunotherapy comprises inhibitors of one or more immune checkpoints. The term “immune checkpoint” refers to a group of molecules on the cell surface of CD4+ and/or CD8+ T cells that fine-tune immune responses by down-modulating or inhibiting an anti-tumor immune response. Immune checkpoint proteins are well-known in the art and include, without limitation, CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRPalpha (CD47), CD48, 2B4 (CD244), B7.1, B7.2, ILT-2, ILT-4, TIGIT, HHLA2, butyrophilins, and A2aR (see, for example, WO 2012/177624). The term further encompasses biologically active protein fragment, as well as nucleic acids encoding full-length immune checkpoint proteins and biologically active protein fragments thereof. In some embodiment, the term further encompasses any fragment according to homology descriptions provided herein. In one embodiment, the immune checkpoint is PD-1.

Immune checkpoints and their sequences are well-known in the art and representative embodiments are described below. For example, the term “PD-1” refers to a member of the immunoglobulin gene superfamily that functions as a coinhibitory receptor having PD-L1 and PD-L2 as known ligands. PD-1 was previously identified using a subtraction cloning based approach to select for genes upregulated during TCR-induced activated T cell death. PD-1 is a member of the CD28/CTLA-4 family of molecules based on its ability to bind to PD-L1. Like CTLA-4, PD-1 is rapidly induced on the surface of T-cells in response to anti-CD3 (Agata et al. 25 (1996) Int. Immunol. 8:765). In contrast to CTLA-4, however, PD-1 is also induced on the surface of B-cells (in response to anti-IgM). PD-1 is also expressed on a subset of thymocytes and myeloid cells (Agata et al. (1996) supra; Nishimura et al. (1996) Int. Immunol. 8:773).

The nucleic acid and amino acid sequences of a representative human PD-1 biomarker is available to the public at the GenBank database under NM_005018.2 and NP_005009.2 (see also Ishida et al. (1992) 20 EMBO J 11:3887; Shinohara et al. (1994) Genomics 23:704; U.S. Pat. No. 5,698,520). PD-1 has an extracellular region containing immunoglobulin superfamily domain, a transmembrane domain, and an intracellular region including an immunoreceptor tyrosine-based inhibitory motif (ITIM) (Ishida et al. (1992) EMBO J. 11:3887; Shinohara et al. (1994) Genomics 23:704; and U.S. Pat. No. 5,698,520) and an immunoreceptor tyrosine-based switch motif (ITSM). These features also define a larger family of polypeptides, called the immunoinhibitory receptors, which also includes gp49B, PIR-B, and the killer inhibitory receptors (KIRs) (Vivier and Daeron (1997) Immunol. Today 18:286). It is often assumed that the tyrosyl phosphorylated ITIM and ITSM motif of these receptors interacts with SH2-domain containing phosphatases, which leads to inhibitory signals. A subset of these immunoinhibitory receptors bind to MHC polypeptides, for example the KIRs, and CTLA4 binds to B7-1 and B7-2. It has been proposed that there is a phylogenetic relationship between the MHC and B7 genes (Henry et al. (1999) Immunol. Today 20(6):285-8). Nucleic acid and polypeptide sequences of PD-1 orthologs in organisms other than humans are well-known and include, for example, rat PD-1 (NM_001106927.1 and NP_001100397.1), dog PD-1 (XM_543338.3 and XP_543338.3), cow PD-1 (NM_001083506.1 and NP_001076975.1), and chicken PD-1 (XM_422723.3 and XP_422723.2).

PD-1 polypeptides are inhibitory receptors capable of transmitting an inhibitory signal to an immune cell to thereby inhibit immune cell effector function, or are capable of promoting costimulation (e.g., by competitive inhibition) of immune cells, e.g., when present in soluble, monomeric form. Preferred PD-1 family members share sequence identity with PD-1 and bind to one or more B7 family members, e.g., B7-1, B7-2, PD-1 ligand, and/or other polypeptides on antigen presenting cells.

The term “PD-1 activity,” includes the ability of a PD-1 polypeptide to modulate an inhibitory signal in an activated immune cell, e.g., by engaging a natural PD-1 ligand on an antigen presenting cell. Modulation of an inhibitory signal in an immune cell results in modulation of proliferation of, and/or cytokine secretion by, an immune cell. Thus, the term “PD-1 activity” includes the ability of a PD-1 polypeptide to bind its natural ligand(s), the ability to modulate immune cell costimulatory or inhibitory signals, and the ability to modulate the immune response.

The term “PD-1 ligand” refers to binding partners of the PD-1 receptor and includes both PD-L1 (Freeman et al. (2000) J. Exp. Med. 192:1027-1034) and PD-L2 (Latchman et al. (2001) Nat. Immunol. 2:261). At least two types of human PD-1 ligand polypeptides exist. PD-1 ligand proteins comprise a signal sequence, and an IgV domain, an IgC domain, a transmembrane domain, and a short cytoplasmic tail. Both PD-L1 (See Freeman et al. (2000) for sequence data) and PD-L2 (See Latchman et al. (2001) Nat. Immunol. 2:261 for sequence data) are members of the B7 family of polypeptides. Both PD-L1 and PD-L2 are expressed in placenta, spleen, lymph nodes, thymus, and heart. Only PD-L2 is expressed in pancreas, lung and liver, while only PD-L1 is expressed in fetal liver. Both PD-1 ligands are upregulated on activated monocytes and dendritic cells, although PD-L1 expression is broader. For example, PD-L1 is known to be constitutively expressed and upregulated to higher levels on murine hematopoietic cells (e.g., T cells, B cells, macrophages, dendritic cells (DCs), and bone marrow-derived mast cells) and non-hematopoietic cells (e.g., endothelial, epithelial, and muscle cells), whereas PD-L2 is inducibly expressed on DCs, macrophages, and bone marrow-derived mast cells (see Butte et al. (2007) Immunity 27:111).

PD-1 ligands comprise a family of polypeptides having certain conserved structural and functional features. The term “family” when used to refer to proteins or nucleic acid molecules, is intended to mean two or more proteins or nucleic acid molecules having a common structural domain or motif and having sufficient amino acid or nucleotide sequence homology, as defined herein. Such family members can be naturally or non-naturally occurring and can be from either the same or different species. For example, a family can contain a first protein of human origin, as well as other, distinct proteins of human origin or alternatively, can contain homologues of non-human origin. Members of a family may also have common functional characteristics. PD-1 ligands are members of the B7 family of polypeptides. The term “B7 family” or “B7 polypeptides” as used herein includes costimulatory polypeptides that share sequence homology with B7 polypeptides, e.g., with B7-1, B7-2, B7h (Swallow et al. (1999) Immunity 11:423), and/or PD-1 ligands (e.g., PD-L1 or PD-L2). For example, human B7-1 and B7-2 share approximately 26% amino acid sequence identity when compared using the BLAST program at NCBI with the default parameters (Blosum62 matrix with gap penalties set at existence 11 and extension 1 (See the NCBI website). The term B7 family also includes variants of these polypeptides which are capable of modulating immune cell function. The B7 family of molecules share a number of conserved regions, including signal domains, IgV domains and the IgC domains. IgV domains and the IgC domains are art-recognized Ig superfamily member domains. These domains correspond to structural units that have distinct folding patterns called Ig folds. Ig folds are comprised of a sandwich of two β sheets, each consisting of anti-parallel β strands of 5-10 amino acids with a conserved disulfide bond between the two sheets in most, but not all, IgC domains of Ig, TCR, and MHC molecules share the same types of sequence patterns and are called the C1-set within the Ig superfamily. Other IgC domains fall within other sets. IgV domains also share sequence patterns and are called V set domains. IgV domains are longer than IgC domains and contain an additional pair of β strands.

Preferred B7 polypeptides are capable of providing costimulatory or inhibitory signals to immune cells to thereby promote or inhibit immune cell responses. For example, B7 family members that bind to costimulatory receptors increase T cell activation and proliferation, while B7 family members that bind to inhibitory receptors reduce costimulation. Moreover, the same B7 family member may increase or decrease T cell costimulation. For example, when bound to a costimulatory receptor, PD-1 ligand can induce costimulation of immune cells or can inhibit immune cell costimulation, e.g., when present in soluble form. When bound to an inhibitory receptor, PD-1 ligand polypeptides can transmit an inhibitory signal to an immune cell. Preferred B7 family members include B7-1, B7-2, B7h, PD-L1 or PD-L2 and soluble fragments or derivatives thereof. In one embodiment, B7 family members bind to one or more receptors on an immune cell, e.g., CTLA4, CD28, ICOS, PD-1 and/or other receptors, and, depending on the receptor, have the ability to transmit an inhibitory signal or a costimulatory signal to an immune cell, preferably a T cell.

Modulation of a costimulatory signal results in modulation of effector function of an immune cell. Thus, the term “PD-1 ligand activity” includes the ability of a PD-1 ligand polypeptide to bind its natural receptor(s) (e.g. PD-1 or B7-1), the ability to modulate immune cell costimulatory or inhibitory signals, and the ability to modulate the immune response.

The term “PD-L1” refers to a specific PD-1 ligand. Two forms of human PD-L1 molecules have been identified. One form is a naturally occurring PD-L1 soluble polypeptide, i.e., having a short hydrophilic domain and no transmembrane domain, and is referred to herein as PD-L1S. The second form is a cell-associated polypeptide, i.e., having a transmembrane and cytoplasmic domain, referred to herein as PD-L1M. The nucleic acid and amino acid sequences of representative human PD-L1 biomarkers regarding PD-L1M are also available to the public at the GenBank database under NM_014143.3 and NP_054862.1. PD-L1 proteins comprise a signal sequence, and an IgV domain and an IgC domain. The signal sequence of PD-L1S is from about amino acid 1 to about amino acid 18. The signal sequence of PD-L1M is from about amino acid 1 to about amino acid 18. The IgV domain of PD-L1S is from about amino acid 19 to about amino acid 134 and the IgV domain of PD-L1M is from about amino acid 19 to about amino acid 134. The IgC domain of PD-L1S is from about amino acid 135 to about amino acid 227 and the IgC domain of PD-L1M is from about amino acid 135 to about amino acid 227. The hydrophilic tail of the PD-L1 exemplified in PD-L1S comprises a hydrophilic tail shown from about amino acid 228 to about amino acid 245. The PD-L1 polypeptide of PD-L1M comprises a transmembrane domain from about amino acids 239 to about amino acid 259 of PD-L1M and a cytoplasmic domain shown from about amino acid 260 to about amino acid 290 of PD-L1M. In addition, nucleic acid and polypeptide sequences of PD-L1 orthologs in organisms other than humans are well-known and include, for example, rat PD-L1 (NM_00191954.1 and NP_001178883.1), dog PD-L1 (XM_541302.3 and XP_541302.3), cow PD-L1 (NM_001163412.1 and NP_001156884.1), and chicken PD-L1 (XM_424811.3 and XP_424811.3).

The term “PD-L2” refers to another specific PD-1 ligand. PD-L2 is a B7 family member expressed on various APCs, including dendritic cells, macrophages and bone-marrow derived mast cells (Zhong et al. (2007) Eur. J. Immunol. 37:2405). APC-expressed PD-L2 is able to both inhibit T cell activation through ligation of PD-1 and costimulate T cell activation, through a PD-1 independent mechanism (Shin et al. (2005) J. Exp. Med. 201:1531). In addition, ligation of dendritic cell-expressed PD-L2 results in enhanced dendritic cell cytokine expression and survival (Radhakrishnan et al. (2003) J. Immunol. 37:1827; Nguyen et al. (2002) J. Exp. Med. 196:1393). The nucleic acid and amino acid sequences of representative human PD-L2 biomarkers are well-known in the art and are also available to the public at the GenBank database under NM_025239.3 and NP_079515.2. PD-L2 proteins are characterized by common structural elements. In some embodiments, PD-L2 proteins include at least one or more of the following domains: a signal peptide domain, a transmembrane domain, an IgV domain, an IgC domain, an extracellular domain, a transmembrane domain, and a cytoplasmic domain. For example, amino acids 1-19 of PD-L2 comprises a signal sequence. As used herein, a “signal sequence” or “signal peptide” serves to direct a polypeptide containing such a sequence to a lipid bilayer, and is cleaved in secreted and membrane bound polypeptides and includes a peptide containing about 15 or more amino acids which occurs at the N-terminus of secretory and membrane bound polypeptides and which contains a large number of hydrophobic amino acid residues. For example, a signal sequence contains at least about 10-30 amino acid residues, preferably about 15-25 amino acid residues, more preferably about 18-20 amino acid residues, and even more preferably about 19 amino acid residues, and has at least about 35-65%, preferably about 38-50%, and more preferably about 40-45% hydrophobic amino acid residues (e.g., valine, leucine, isoleucine or phenylalanine). In another embodiment, amino acid residues 220-243 of the native human PD-L2 polypeptide and amino acid residues 201-243 of the mature polypeptide comprise a transmembrane domain. As used herein, the term “transmembrane domain” includes an amino acid sequence of about 15 amino acid residues in length which spans the plasma membrane. More preferably, a transmembrane domain includes about at least 20, 25, 30, 35, 40, or 45 amino acid residues and spans the plasma membrane. Transmembrane domains are rich in hydrophobic residues, and typically have an alpha-helical structure. In a preferred embodiment, at least 50%, 60%, 70%, 80%, 90%, 95% or more of the amino acids of a transmembrane domain are hydrophobic, e.g., leucines, isoleucines, tyrosines, or tryptophans. Transmembrane domains are described in, for example, Zagotta, W. N. et al. (1996) Annu. Rev. Neurosci. 19: 235-263. In still another embodiment, amino acid residues 20-120 of the native human PD-L2 polypeptide and amino acid residues 1-101 of the mature polypeptide comprise an IgV domain. Amino acid residues 121-219 of the native human PD-L2 polypeptide and amino acid residues 102-200 of the mature polypeptide comprise an IgC domain. As used herein, IgV and IgC domains are recognized in the art as Ig superfamily member domains. These domains correspond to structural units that have distinct folding patterns called Ig folds. Ig folds are comprised of a sandwich of two β sheets, each consisting of antiparallel (3 strands of 5-10 amino acids with a conserved disulfide bond between the two sheets in most, but not all, domains. IgC domains of Ig, TCR, and MHC molecules share the same types of sequence patterns and are called the Cl set within the Ig superfamily. Other IgC domains fall within other sets. IgV domains also share sequence patterns and are called V set domains. IgV domains are longer than C-domains and form an additional pair of strands. In yet another embodiment, amino acid residues 1-219 of the native human PD-L2 polypeptide and amino acid residues 1-200 of the mature polypeptide comprise an extracellular domain. As used herein, the term “extracellular domain” represents the N-terminal amino acids which extend as a tail from the surface of a cell. An extracellular domain of the present invention includes an IgV domain and an IgC domain, and may include a signal peptide domain. In still another embodiment, amino acid residues 244-273 of the native human PD-L2 polypeptide and amino acid residues 225-273 of the mature polypeptide comprise a cytoplasmic domain. As used herein, the term “cytoplasmic domain” represents the C-terminal amino acids which extend as a tail into the cytoplasm of a cell. In addition, nucleic acid and polypeptide sequences of PD-L2 orthologs in organisms other than humans are well-known and include, for example, rat PD-L2 (NM_001107582.2 and NP_001101052.2), dog PD-L2 (XM_847012.2 and XP_852105.2), cow PD-L2 (XM_586846.5 and XP_586846.3), and chimpanzee PD-L2 (XM_001140776.2 and XP_001140776.1).

The term “PD-L2 activity,” “biological activity of PD-L2,” or “functional activity of PD-L2,” refers to an activity exerted by a PD-L2 protein, polypeptide or nucleic acid molecule on a PD-L2-responsive cell or tissue, or on a PD-L2 polypeptide binding partner, as determined in vivo, or in vitro, according to standard techniques. In one embodiment, a PD-L2 activity is a direct activity, such as an association with a PD-L2 binding partner. As used herein, a “target molecule” or “binding partner” is a molecule with which a PD-L2 polypeptide binds or interacts in nature, such that PD-L2-mediated function is achieved. In an exemplary embodiment, a PD-L2 target molecule is the receptor RGMb. Alternatively, a PD-L2 activity is an indirect activity, such as a cellular signaling activity mediated by interaction of the PD-L2 polypeptide with its natural binding partner (i.e., physiologically relevant interacting macromolecule involved in an immune function or other biologically relevant function), e.g., RGMb. The biological activities of PD-L2 are described herein. For example, the PD-L2 polypeptides of the present invention can have one or more of the following activities: 1) bind to and/or modulate the activity of the receptor RGMb, PD-1, or other PD-L2 natural binding partners, 2) modulate intra- or intercellular signaling, 3) modulate activation of immune cells, e.g., T lymphocytes, and 4) modulate the immune response of an organism, e.g., a human organism.

“Anti-immune checkpoint therapy” refers to the use of agents that inhibit immune checkpoint nucleic acids and/or proteins. Inhibition of one or more immune checkpoints can block or otherwise neutralize inhibitory signaling to thereby upregulate an immune response in order to more efficaciously treat cancer. Exemplary agents useful for inhibiting immune checkpoints include antibodies, small molecules, peptides, peptidomimetics, natural ligands, and derivatives of natural ligands, that can either bind and/or inactivate or inhibit immune checkpoint proteins, or fragments thereof; as well as RNA interference, antisense, nucleic acid aptamers, etc. that can downregulate the expression and/or activity of immune checkpoint nucleic acids, or fragments thereof. Exemplary agents for upregulating an immune response include antibodies against one or more immune checkpoint proteins block the interaction between the proteins and its natural receptor(s); a non-activating form of one or more immune checkpoint proteins (e.g., a dominant negative polypeptide); small molecules or peptides that block the interaction between one or more immune checkpoint proteins and its natural receptor(s); fusion proteins (e.g. the extracellular portion of an immune checkpoint inhibition protein fused to the Fc portion of an antibody or immunoglobulin) that bind to its natural receptor(s); nucleic acid molecules that block immune checkpoint nucleic acid transcription or translation; and the like. Such agents can directly block the interaction between the one or more immune checkpoints and its natural receptor(s) (e.g., antibodies) to prevent inhibitory signaling and upregulate an immune response. Alternatively, agents can indirectly block the interaction between one or more immune checkpoint proteins and its natural receptor(s) to prevent inhibitory signaling and upregulate an immune response. For example, a soluble version of an immune checkpoint protein ligand such as a stabilized extracellular domain can binding to its receptor to indirectly reduce the effective concentration of the receptor to bind to an appropriate ligand. In one embodiment, anti-PD-1 antibodies, anti-PD-L1 antibodies, and/or anti-PD-L2 antibodies, either alone or in combination, are used to inhibit immune checkpoints. These embodiments are also applicable to specific therapy against particular immune checkpoints, such as the PD-1 pathway (e.g., anti-PD-1 pathway therapy, otherwise known as PD-1 pathway inhibitor therapy).

The term “immune response” includes T cell mediated and/or B cell mediated immune responses. Exemplary immune responses include T cell responses, e.g., cytokine production and cellular cytotoxicity. In addition, the term immune response includes immune responses that are indirectly effected by T cell activation, e.g., antibody production (humoral responses) and activation of cytokine responsive cells, e.g., macrophages.

The term “immunotherapeutic agent” can include any molecule, peptide, antibody or other agent which can stimulate a host immune system to generate an immune response to a tumor or cancer in the subject. Various immunotherapeutic agents are useful in the compositions and methods described herein.

The term “inhibit” includes decreasing, reducing, limiting, and/or blocking, of, for example a particular action, function, and/or interaction. In some embodiments, the interation between two molecules is “inhibited” if the interaction is reduced, blocked, disrupted or destablized.

In some embodiments, cancer is “inhibited” if at least one symptom of the cancer is alleviated, terminated, slowed, or prevented. As used herein, cancer is also “inhibited” if recurrence or metastasis of the cancer is reduced, slowed, delayed, or prevented.

The term “interaction”, when referring to an interaction between two molecules, refers to the physical contact (e.g., binding) of the molecules with one another. Generally, such an interaction results in an activity (which produces a biological effect) of one or both of said molecules.

An “isolated protein” refers to a protein that is substantially free of other proteins, cellular material, separation medium, and culture medium when isolated from cells or produced by recombinant DNA techniques, or chemical precursors or other chemicals when chemically synthesized. An “isolated” or “purified” protein or biologically active portion thereof is substantially free of cellular material or other contaminating proteins from the cell or tissue source from which the antibody, polypeptide, peptide or fusion protein is derived, or substantially free from chemical precursors or other chemicals when chemically synthesized. The language “substantially free of cellular material” includes preparations of a biomarker polypeptide or fragment thereof, in which the protein is separated from cellular components of the cells from which it is isolated or recombinantly produced. In one embodiment, the language “substantially free of cellular material” includes preparations of a biomarker protein or fragment thereof, having less than about 30% (by dry weight) of non-biomarker protein (also referred to herein as a “contaminating protein”), more preferably less than about 20% of non-biomarker protein, still more preferably less than about 10% of non-biomarker protein, and most preferably less than about 5% non-biomarker protein. When antibody, polypeptide, peptide or fusion protein or fragment thereof, e.g., a biologically active fragment thereof, is recombinantly produced, it is also preferably substantially free of culture medium, i.e., culture medium represents less than about 20%, more preferably less than about 10%, and most preferably less than about 5% of the volume of the protein preparation.

As used herein, the term “isotype” refers to the antibody class (e.g., IgM, IgG1, IgG2C, and the like) that is encoded by heavy chain constant region genes.

The “normal” level of expression of a biomarker is the level of expression of the biomarker in cells of a subject, e.g., a human patient, not afflicted with a cancer. An “over-expression” or “significantly higher level of expression” of a biomarker refers to an expression level in a test sample that is greater than the standard error of the assay employed to assess expression, and is preferably at least 10%, and more preferably 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more higher than the expression activity or level of the biomarker in a control sample (e.g., sample from a healthy subject not having the biomarker associated disease) and preferably, the average expression level of the biomarker in several control samples. A “significantly lower level of expression” of a biomarker refers to an expression level in a test sample that is at least 10%, and more preferably 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more lower than the expression level of the biomarker in a control sample (e.g., sample from a healthy subject not having the biomarker associated disease) and preferably, the average expression level of the biomarker in several control samples.

An “over-expression” or “significantly higher level of expression” of a biomarker refers to an expression level in a test sample that is greater than the standard error of the assay employed to assess expression, and is preferably at least 10%, and more preferably 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more higher than the expression activity or level of the biomarker in a control sample (e.g., sample from a healthy subject not having the biomarker associated disease) and preferably, the average expression level of the biomarker in several control samples. A “significantly lower level of expression” of a biomarker refers to an expression level in a test sample that is at least 10%, and more preferably 1.2, 1.3, 1.4, 1.5, 1.6, 1.7, 1.8, 1.9, 2.0, 2.1, 2.1, 2.2, 2.3, 2.4, 2.5, 2.6, 2.7, 2.8, 2.9, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, 20 times or more lower than the expression level of the biomarker in a control sample (e.g., sample from a healthy subject not having the biomarker associated disease) and preferably, the average expression level of the biomarker in several control samples.

The term “predictive” includes the use of a biomarker nucleic acid and/or protein status, e.g., over- or under-activity, emergence, expression, growth, remission, recurrence or resistance of tumors before, during or after therapy, for determining the likelihood of response of a cancer to a cancer vaccine alone or in combination with an immunotherapy and/or cancer therapy. Such predictive use of the biomarker may be confirmed by, e.g., (1) increased or decreased copy number (e.g., by FISH, FISH plus SKY, single-molecule sequencing, e.g., as described in the art at least at J. Biotechnol., 86:289-301, or qPCR), overexpression or underexpression of a biomarker nucleic acid (e.g., by ISH, Northern Blot, or qPCR), increased or decreased biomarker protein (e.g., by IHC), or increased or decreased activity, e.g., in more than about 5%, 6%, 7%, 8%, 9%, 10%, 11%, 12%, 13%, 14%, 15%, 20%, 25%, 30%, 40%, 50%, 60%, 70%, 80%, 90%, 95%, 100%, or more of assayed human cancers types or cancer samples; (2) its absolute or relatively modulated presence or absence in a biological sample, e.g., a sample containing tissue, whole blood, serum, plasma, buccal scrape, saliva, cerebrospinal fluid, urine, stool, or bone marrow, from a subject, e.g. a human, afflicted with cancer; (3) its absolute or relatively modulated presence or absence in clinical subset of patients with cancer (e.g., those responding to the cancer vaccine alone or in combination with an immunotherapy and/or cancer therapy, or those developing resistance thereto).

The terms “prevent,” “preventing,” “prevention,” “prophylactic treatment,” and the like refer to reducing the probability of developing a disease, disorder, or condition in a subject, who does not have, but is at risk of or susceptible to developing a disease, disorder, or condition.

The term “cancer response,” “response to immunotherapy,” or “response to modulators of T-cell mediated cytotoxicity/immunotherapy combination therapy” relates to any response of the hyperproliferative disorder (e.g., cancer) to a cancer agent, such as a modulator of T-cell mediated cytotoxicity, and an immunotherapy, preferably to a change in tumor mass and/or volume after initiation of neoadjuvant or adjuvant therapy. Hyperproliferative disorder response may be assessed, for example for efficacy or in a neoadjuvant or adjuvant situation, where the size of a tumor after systemic intervention can be compared to the initial size and dimensions as measured by CT, PET, mammogram, ultrasound or palpation. Responses may also be assessed by caliper measurement or pathological examination of the tumor after biopsy or surgical resection. Response may be recorded in a quantitative fashion like percentage change in tumor volume or in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. Assessment of hyperproliferative disorder response may be done early after the onset of neoadjuvant or adjuvant therapy, e.g., after a few hours, days, weeks or preferably after a few months. A typical endpoint for response assessment is upon termination of neoadjuvant chemotherapy or upon surgical removal of residual tumor cells and/or the tumor bed. This is typically three months after initiation of neoadjuvant therapy. In some embodiments, clinical efficacy of the therapeutic treatments described herein may be determined by measuring the clinical benefit rate (CBR). The clinical benefit rate is measured by determining the sum of the percentage of patients who are in complete remission (CR), the number of patients who are in partial remission (PR) and the number of patients having stable disease (SD) at a time point at least 6 months out from the end of therapy. The shorthand for this formula is CBR=CR+PR+SD over 6 months. In some embodiments, the CBR for a particular cancer therapeutic regimen is at least 25%, 30%, 35%, 40%, 45%, 50%, 55%, 60%, 65%, 70%, 75%, 80%, 85%, or more. Additional criteria for evaluating the response to cancer therapies are related to “survival,” which includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g., time of diagnosis or start of treatment) and end point (e.g., death, recurrence or metastasis). In addition, criteria for efficacy of treatment can be expanded to include response to chemotherapy, probability of survival, probability of metastasis within a given time period, and probability of tumor recurrence. For example, in order to determine appropriate threshold values, a particular cancer therapeutic regimen can be administered to a population of subjects and the outcome can be correlated to biomarker measurements that were determined prior to administration of any cancer therapy. The outcome measurement may be pathologic response to therapy given in the neoadjuvant setting. Alternatively, outcome measures, such as overall survival and disease-free survival can be monitored over a period of time for subjects following cancer therapy for which biomarker measurement values are known. In certain embodiments, the doses administered are standard doses known in the art for cancer therapeutic agents. The period of time for which subjects are monitored can vary. For example, subjects may be monitored for at least 2, 4, 6, 8, 10, 12, 14, 16, 18, 20, 25, 30, 35, 40, 45, 50, 55, or 60 months. Biomarker measurement threshold values that correlate to outcome of a cancer therapy can be determined using well-known methods in the art, such as those described in the Examples section.

The term “resistance” refers to an acquired or natural resistance of a cancer sample or a mammal to a cancer therapy (i.e., being nonresponsive to or having reduced or limited response to the therapeutic treatment), such as having a reduced response to a therapeutic treatment by 5%, 10%, 15%, 20%, 25%, 30%, 35%, 40%, 45%, 50%, 55%, 60%, 65%, 70%, 75%, 80%, 85%, 90%, 95%, 100%, or more, such 2-fold, 3-fold, 4-fold, 5-fold, 10-fold, 15-fold, 20-fold or more, or any range in between, inclusive. The reduction in response can be measured by comparing with the same cancer sample or mammal before the resistance is acquired, or by comparing with a different cancer sample or a mammal that is known to have no resistance to the therapeutic treatment. A typical acquired resistance to chemotherapy is called “multidrug resistance.” The multidrug resistance can be mediated by P-glycoprotein or can be mediated by other mechanisms, or it can occur when a mammal is infected with a multi-drug-resistant microorganism or a combination of microorganisms. The determination of resistance to a therapeutic treatment is routine in the art and within the skill of an ordinarily skilled clinician, for example, can be measured by cell proliferative assays and cell death assays as described herein as “sensitizing.” In some embodiments, the term “reverses resistance” means that the use of a second agent in combination with a primary cancer therapy (e.g., chemotherapeutic or radiation therapy) is able to produce a significant decrease in tumor volume at a level of statistical significance (e.g., p<0.05) when compared to tumor volume of untreated tumor in the circumstance where the primary cancer therapy (e.g., chemotherapeutic or radiation therapy) alone is unable to produce a statistically significant decrease in tumor volume compared to tumor volume of untreated tumor. This generally applies to tumor volume measurements made at a time when the untreated tumor is growing log rhythmically.

The terms “response” or “responsiveness” refers to a cancer response, e.g. in the sense of reduction of tumor size or inhibiting tumor growth. The terms can also refer to an improved prognosis, for example, as reflected by an increased time to recurrence, which is the period to first recurrence censoring for second primary cancer as a first event or death without evidence of recurrence, or an increased overall survival, which is the period from treatment to death from any cause. To respond or to have a response means there is a beneficial endpoint attained when exposed to a stimulus. Alternatively, a negative or detrimental symptom is minimized, mitigated or attenuated on exposure to a stimulus. It will be appreciated that evaluating the likelihood that a tumor or subject will exhibit a favorable response is equivalent to evaluating the likelihood that the tumor or subject will not exhibit favorable response (i.e., will exhibit a lack of response or be non-responsive).

An “RNA interfering agent” as used herein, is defined as any agent which interferes with or inhibits expression of a target biomarker gene by RNA interference (RNAi). Such RNA interfering agents include, but are not limited to, nucleic acid molecules including RNA molecules which are homologous to the target biomarker gene of the present invention, or a fragment thereof, short interfering RNA (siRNA), and small molecules which interfere with or inhibit expression of a target biomarker nucleic acid by RNA interference (RNAi).

“RNA interference (RNAi)” is an evolutionally conserved process whereby the expression or introduction of RNA of a sequence that is identical or highly similar to a target biomarker nucleic acid results in the sequence specific degradation or specific post-transcriptional gene silencing (PTGS) of messenger RNA (mRNA) transcribed from that targeted gene (see Coburn and Cullen (2002) J. Virol. 76:9225), thereby inhibiting expression of the target biomarker nucleic acid. In one embodiment, the RNA is double stranded RNA (dsRNA). This process has been described in plants, invertebrates, and mammalian cells. In nature, RNAi is initiated by the dsRNA-specific endonuclease Dicer, which promotes processive cleavage of long dsRNA into double-stranded fragments termed siRNAs. siRNAs are incorporated into a protein complex that recognizes and cleaves target mRNAs. RNAi can also be initiated by introducing nucleic acid molecules, e.g., synthetic siRNAs or RNA interfering agents, to inhibit or silence the expression of target biomarker nucleic acids. As used herein, “inhibition of target biomarker nucleic acid expression” or “inhibition of marker gene expression” includes any decrease in expression or protein activity or level of the target biomarker nucleic acid or protein encoded by the target biomarker nucleic acid. The decrease may be of at least 30%, 40%, 50%, 60%, 70%, 80%, 90%, 95% or 99% or more as compared to the expression of a target biomarker nucleic acid or the activity or level of the protein encoded by a target biomarker nucleic acid which has not been targeted by an RNA interfering agent.

In addition to RNAi, genome editing can be used to modulate the copy number or genetic sequence of a biomarker of interest, such as constitutive or induced knockout or mutation of a biomarker of interest. For example, the CRISPR-Cas system can be used for precise editing of genomic nucleic acids (e.g., for creating non-functional or null mutations). In such embodiments, the CRISPR guide RNA and/or the Cas enzyme may be expressed. For example, a vector containing only the guide RNA can be administered to an animal or cells transgenic for the Cas9 enzyme. Similar strategies may be used (e.g., designer zinc finger, transcription activator-like effectors (TALEs) or homing meganucleases). Such systems are well-known in the art (see, for example, U.S. Pat. No. 8,697,359; Sander and Joung (2014) Nat. Biotech. 32:347-355; Hale et al. (2009) Cell 139:945-956; Karginov and Hannon (2010) Mol. Cell 37:7; U.S. Pat. Publ. 2014/0087426 and 2012/0178169; Boch et al. (2011) Nat. Biotech. 29:135-136; Boch et al. (2009) Science 326:1509-1512; Moscou and Bogdanove (2009) Science 326:1501; Weber et al. (2011) PLoS One 6:e19722; Li et al. (2011) Nucl. Acids Res. 39:6315-6325; Zhang et al. (2011) Nat. Biotech. 29:149-153; Miller et al. (2011) Nat. Biotech. 29:143-148; Lin et al. (2014) Nucl. Acids Res. 42:e47). Such genetic strategies can use constitutive expression systems or inducible expression systems according to well-known methods in the art.

“Piwi-interacting RNA (piRNA)” is the largest class of small non-coding RNA molecules. piRNAs form RNA-protein complexes through interactions with piwi proteins. These piRNA complexes have been linked to both epigenetic and post-transcriptional gene silencing of retrotransposons and other genetic elements in germ line cells, particularly those in spermatogenesis. They are distinct from microRNA (miRNA) in size (26-31 nt rather than 21-24 nt), lack of sequence conservation, and increased complexity. However, like other small RNAs, piRNAs are thought to be involved in gene silencing, specifically the silencing of transposons. The majority of piRNAs are antisense to transposon sequences, suggesting that transposons are the piRNA target. In mammals it appears that the activity of piRNAs in transposon silencing is most important during the development of the embryo, and in both C. elegans and humans, piRNAs are necessary for spermatogenesis. piRNA has a role in RNA silencing via the formation of an RNA-induced silencing complex (RISC).

“Aptamers” are oligonucleotide or peptide molecules that bind to a specific target molecule. “Nucleic acid aptamers” are nucleic acid species that have been engineered through repeated rounds of in vitro selection or equivalently, SELEX (systematic evolution of ligands by exponential enrichment) to bind to various molecular targets such as small molecules, proteins, nucleic acids, and even cells, tissues and organisms. “Peptide aptamers” are artificial proteins selected or engineered to bind specific target molecules. These proteins consist of one or more peptide loops of variable sequence displayed by a protein scaffold. They are typically isolated from combinatorial libraries and often subsequently improved by directed mutation or rounds of variable region mutagenesis and selection. The “Affimer protein”, an evolution of peptide aptamers, is a small, highly stable protein engineered to display peptide loops which provides a high affinity binding surface for a specific target protein. It is a protein of low molecular weight, 12-14 kDa, derived from the cysteine protease inhibitor family of cystatins. Aptamers are useful in biotechnological and therapeutic applications as they offer molecular recognition properties that rival that of the commonly used biomolecule, antibodies. In addition to their discriminate recognition, aptamers offer advantages over antibodies as they can be engineered completely in a test tube, are readily produced by chemical synthesis, possess desirable storage properties, and elicit little or no immunogenicity in therapeutic applications.

As used herein, the term “intracellular immunoglobulin molecule” is a complete immunoglobulin which is the same as a naturally-occurring secreted immunoglobulin, but which remains inside of the cell following synthesis. An “intracellular immunoglobulin fragment” refers to any fragment, including single-chain fragments of an intracellular immunoglobulin molecule. Thus, an intracellular immunoglobulin molecule or fragment thereof is not secreted or expressed on the outer surface of the cell. Single-chain intracellular immunoglobulin fragments are referred to herein as “single-chain immunoglobulins.” As used herein, the term “intracellular immunoglobulin molecule or fragment thereof” is understood to encompass an “intracellular immunoglobulin,” a “single-chain intracellular immunoglobulin” (or fragment thereof), an “intracellular immunoglobulin fragment,” an “intracellular antibody” (or fragment thereof), and an “intrabody” (or fragment thereof). As such, the terms “intracellular immunoglobulin,” “intracellular Ig,” “intracellular antibody,” and “intrabody” may be used interchangeably herein, and are all encompassed by the generic definition of an “intracellular immunoglobulin molecule, or fragment thereof.” An intracellular immunoglobulin molecule, or fragment thereof of the present invention may, in some embodiments, comprise two or more subunit polypeptides, e.g., a “first intracellular immunoglobulin subunit polypeptide” and a “second intracellular immunoglobulin subunit polypeptide.” However, in other embodiments, an intracellular immunoglobulin may be a “single-chain intracellular immunoglobulin” including only a single polypeptide. As used herein, a “single-chain intracellular immunoglobulin” is defined as any unitary fragment that has a desired activity, for example, intracellular binding to an antigen. Thus, single-chain intracellular immunoglobulins encompass those which comprise both heavy and light chain variable regions which act together to bind antigen, as well as single-chain intracellular immunoglobulins which only have a single variable region which binds antigen, for example, a “camelized” heavy chain variable region as described herein. An intracellular immunoglobulin or Ig fragment may be expressed anywhere substantially within the cell, such as in the cytoplasm, on the inner surface of the cell membrane, or in a subcellular compartment (also referred to as cell subcompartment or cell compartment) such as the nucleus, Golgi, endoplasmic reticulum, endosome, mitochondria, etc. Additional cell subcompartments include those that are described herein and well known in the art.

The term “sample” used for detecting or determining the presence or level of at least one biomarker is typically whole blood, plasma, serum, saliva, urine, stool (e.g., feces), tears, and any other bodily fluid (e.g., as described above under the definition of “body fluids”), or a tissue sample (e.g., biopsy) such as bone marrow and bone sample, or surgical resection tissue. In certain instances, the method of the present invention further comprises obtaining the sample from the individual prior to detecting or determining the presence or level of at least one marker in the sample.

The term “sensitize” means to alter cancer cells or tumor cells in a way that allows for more effective treatment of the associated cancer with a cancer therapy (e.g., anti-immune checkpoint, chemotherapeutic, and/or radiation therapy). In some embodiments, normal cells are not affected to an extent that causes the normal cells to be unduly injured by the therapies. An increased sensitivity or a reduced sensitivity to a therapeutic treatment is measured according to a known method in the art for the particular treatment and methods described herein below, including, but not limited to, cell proliferative assays (Tanigawa N, Kern D H, Kikasa Y, Morton D L, Cancer Res 1982; 42: 2159-2164), cell death assays (Weisenthal L M, Shoemaker R H, Marsden J A, Dill P L, Baker J A, Moran E M, Cancer Res 1984; 94: 161-173; Weisenthal L M, Lippman M E, Cancer Treat Rep 1985; 69: 615-632; Weisenthal L M, In: Kaspers G J L, Pieters R, Twentyman P R, Weisenthal L M, Veerman A J P, eds. Drug Resistance in Leukemia and Lymphoma. Langhorne, P A: Harwood Academic Publishers, 1993: 415-432; Weisenthal L M, Contrib Gynecol Obstet 1994; 19: 82-90). The sensitivity or resistance may also be measured in animal by measuring the tumor size reduction over a period of time, for example, 6 month for human. A composition or a method sensitizes response to a therapeutic treatment if the increase in treatment sensitivity or the reduction in resistance is 5%, 10%, 15%, 20%, 25%, 30%, 35%, 40%, 45%, 50%, 55%, 60%, 65%, 70%, 75%, 80%, 85%, 90%, 95%, 100%, or more, such 2-fold, 3-fold, 4-fold, 5-fold, 10-fold, 15-fold, 20-fold or more, or any range in between, inclusive, compared to treatment sensitivity or resistance in the absence of such composition or method. The determination of sensitivity or resistance to a therapeutic treatment is routine in the art and within the skill of an ordinarily skilled clinician. It is to be understood that any method described herein for enhancing the efficacy of a cancer therapy can be equally applied to methods for sensitizing hyperproliferative or otherwise cancerous cells (e.g., resistant cells) to the cancer therapy.

“Short interfering RNA” (siRNA), also referred to herein as “small interfering RNA” is defined as an agent which functions to inhibit expression of a target biomarker nucleic acid, e.g., by RNAi. An siRNA may be chemically synthesized, may be produced by in vitro transcription, or may be produced within a host cell. In one embodiment, siRNA is a double stranded RNA (dsRNA) molecule of about 15 to about 40 nucleotides in length, preferably about 15 to about 28 nucleotides, more preferably about 19 to about 25 nucleotides in length, and more preferably about 19, 20, 21, or 22 nucleotides in length, and may contain a 3′ and/or 5′ overhang on each strand having a length of about 0, 1, 2, 3, 4, or 5 nucleotides. The length of the overhang is independent between the two strands, i.e., the length of the overhang on one strand is not dependent on the length of the overhang on the second strand. Preferably the siRNA is capable of promoting RNA interference through degradation or specific post-transcriptional gene silencing (PTGS) of the target messenger RNA (mRNA).

In another embodiment, an siRNA is a small hairpin (also called stem loop) RNA (shRNA). In one embodiment, these shRNAs are composed of a short (e.g., 19-25 nucleotide) antisense strand, followed by a 5-9 nucleotide loop, and the analogous sense strand. Alternatively, the sense strand may precede the nucleotide loop structure and the antisense strand may follow. These shRNAs may be contained in plasmids, retroviruses, and lentiviruses and expressed from, for example, the pol III U6 promoter, or another promoter (see, e.g., Stewart, et al. (2003) RNA April; 9(4):493-501 incorporated by reference herein).

RNA interfering agents, e.g., siRNA molecules, may be administered to a patient having or at risk for having cancer, to inhibit expression of a biomarker gene which is overexpressed in cancer and thereby treat, prevent, or inhibit cancer in the subject.

The term “small molecule” is a term of the art and includes molecules that are less than about 1000 molecular weight or less than about 500 molecular weight. In one embodiment, small molecules do not exclusively comprise peptide bonds. In another embodiment, small molecules are not oligomeric. Exemplary small molecule compounds which can be screened for activity include, but are not limited to, peptides, peptidomimetics, nucleic acids, carbohydrates, small organic molecules (e.g., polyketides) (Cane et al. (1998) Science 282:63), and natural product extract libraries. In another embodiment, the compounds are small, organic non-peptidic compounds. In a further embodiment, a small molecule is not biosynthetic.

The term “specific binding” refers to antibody binding to a predetermined antigen. Typically, the antibody binds with an affinity (K_{D}) of approximately less than 10⁻⁷ M, such as approximately less than 10⁻⁸ M, 10⁻⁹ M or 10⁻¹⁰ M or even lower when determined by surface plasmon resonance (SPR) technology in a BIACORE® assay instrument using an antigen of interest as the analyte and the antibody as the ligand, and binds to the predetermined antigen with an affinity that is at least 1.1-, 1.2-, 1.3-, 1.4-, 1.5-, 1.6-, 1.7-, 1.8-, 1.9-, 2.0-, 2.5-, 3.0-, 3.5-, 4.0-, 4.5-, 5.0-, 6.0-, 7.0-, 8.0-, 9.0-, or 10.0-fold or greater than its affinity for binding to a non-specific antigen (e.g., BSA, casein) other than the predetermined antigen or a closely-related antigen. The phrases “an antibody recognizing an antigen” and “an antibody specific for an antigen” are used interchangeably herein with the term “an antibody which binds specifically to an antigen.” Selective binding is a relative term referring to the ability of an antibody to discriminate the binding of one antigen over another.

The term “subject” refers to any healthy animal, mammal or human, or any animal, mammal or human afflicted with a cancer, e.g., brain, lung, ovarian, pancreatic, liver, breast, prostate, and/or colorectal cancers, melanoma, multiple myeloma, and the like. The term “subject” is interchangeable with “patient.”

The term “survival” includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g. time of diagnosis or start of treatment) and end point (e.g. death, recurrence or metastasis). In addition, criteria for efficacy of treatment can be expanded to include response to chemotherapy, probability of survival, probability of metastasis within a given time period, and probability of tumor recurrence.

The term “synergistic effect” refers to the combined effect of two or more cancer agents (e.g., a cancer vaccine in combination with immunotherapy) can be greater than the sum of the separate effects of the cancer agents/therapies alone.

The term “T cell” includes CD4⁺ T cells and CD8⁺ T cells. The term T cell also includes both T helper 1 type T cells and T helper 2 type T cells. The term “antigen presenting cell” includes professional antigen presenting cells (e.g., B lymphocytes, monocytes, dendritic cells, Langerhans cells), as well as other antigen presenting cells (e.g., keratinocytes, endothelial cells, astrocytes, fibroblasts, and oligodendrocytes).

The term “therapeutic effect” refers to a local or systemic effect in animals, particularly mammals, and more particularly humans, caused by a pharmacologically active substance. The term thus means any substance intended for use in the diagnosis, cure, mitigation, treatment or prevention of disease or in the enhancement of desirable physical or mental development and conditions in an animal or human. The phrase “therapeutically-effective amount” means that amount of such a substance that produces some desired local or systemic effect at a reasonable benefit/risk ratio applicable to any treatment. In certain embodiments, a therapeutically effective amount of a compound will depend on its therapeutic index, solubility, and the like. For example, certain compounds discovered by the methods of the present invention may be administered in a sufficient amount to produce a reasonable benefit/risk ratio applicable to such treatment.

The terms “therapeutically-effective amount” and “effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment. Toxicity and therapeutic efficacy of subject compounds may be determined by standard pharmaceutical procedures in cell cultures or experimental animals, e.g., for determining the LD₅₀ and the ED₅₀. Compositions that exhibit large therapeutic indices are preferred. In some embodiments, the LD₅₀ (lethal dosage) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more reduced for the agent relative to no administration of the agent. Similarly, the ED₅₀ (i.e., the concentration which achieves a half-maximal inhibition of symptoms) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more increased for the agent relative to no administration of the agent. Also, Similarly, the IC₅₀ (i.e., the concentration which achieves half-maximal cytotoxic or cytostatic effect on cancer cells) can be measured and can be, for example, at least 10%, 20%, 30%, 40%, 50%, 60%, 70%, 80%, 90%, 100%, 200%, 300%, 400%, 500%, 600%, 700%, 800%, 900%, 1000% or more increased for the agent relative to no administration of the agent. In some embodiments, cancer cell growth in an assay can be inhibited by at least about 10%, 15%, 20%, 25%, 30%, 35%, 40%, 45%, 50%, 55%, 60%, 65%, 70%, 75%, 80%, 85%, 90%, 95%, or even 100%. In another embodiment, at least about a 10%, 15%, 20%, 25%, 30%, 35%, 40%, 45%, 50%, 55%, 60%, 65%, 70%, 75%, 80%, 85%, 90%, 95%, or even 100% decrease in a solid malignancy can be achieved.

A “transcribed polynucleotide” or “nucleotide transcript” is a polynucleotide (e.g. an mRNA, hnRNA, a cDNA, or an analog of such RNA or cDNA) which is complementary to or homologous with all or a portion of a mature mRNA made by transcription of a biomarker nucleic acid and normal post-transcriptional processing (e.g. splicing), if any, of the RNA transcript, and reverse transcription of the RNA transcript.

As used herein, the term “unresponsiveness” includes refractivity of cancer cells to therapy or refractivity of therapeutic cells, such as immune cells, to stimulation, e.g., stimulation via an activating receptor or a cytokine. Unresponsiveness can occur, e.g., because of exposure to immunosuppressants or exposure to high doses of antigen. As used herein, the term “anergy” or “tolerance” includes refractivity to activating receptor-mediated stimulation. Such refractivity is generally antigen-specific and persists after exposure to the tolerizing antigen has ceased. For example, anergy in T cells (as opposed to unresponsiveness) is characterized by lack of cytokine production, e.g., IL-2. T cell anergy occurs when T cells are exposed to antigen and receive a first signal (a T cell receptor or CD-3 mediated signal) in the absence of a second signal (a costimulatory signal). Under these conditions, reexposure of the cells to the same antigen (even if reexposure occurs in the presence of a costimulatory polypeptide) results in failure to produce cytokines and, thus, failure to proliferate. Anergic T cells can, however, proliferate if cultured with cytokines (e.g., IL-2). For example, T cell anergy can also be observed by the lack of IL-2 production by T lymphocytes as measured by ELISA or by a proliferation assay using an indicator cell line. Alternatively, a reporter gene construct can be used. For example, anergic T cells fail to initiate IL-2 gene transcription induced by a heterologous promoter under the control of the 5′ IL-2 gene enhancer or by a multimer of the AP1 sequence that can be found within the enhancer (Kang et al. (1992) Science 257:1134).

The term “STING” or “stimulator of interferon genes”, also known as transmembrane protein 173 (TMEM 173), refers to a five transmembrane protein that functions as a major regulator of the innate immune response to viral and bacterial infections. STING is a cytosolic receptor that senses both exogenous and endogenous cytosolic cyclic dinucleotides (CDNs), activating TBK1/IRF3 (interferon regulatory factor 3), NF-κB (nuclear factor κB), and STAT6 (signal transducer and activator of transcription 6) signaling pathways to induce robust type I interferon and proinflammatory cytokine responses. The term “STING” is intended to include fragments, variants (e.g., allelic variants) and derivatives thereof. Representative human STING cDNA and human STING protein sequences are well-known in the art and are publicly available from the National Center for Biotechnology Information (NCBI). Human STING isoforms include the longer isoform 1 (NM_198282.3 and NP_938023.1), and the shorter isoform 2 (NM_001301738.1 and NP_001288667.1; which has a shorter 5′ UTR and lacks an exon in the 3′ coding region which results in a shorter and distinct C-terminus compared to variant 1). Nucleic acid and polypeptide sequences of STING orthologs in organisms other than humans are well-known and include, for example, chimpanzee CDH1 (XM_016953921.1 and XP_016809410.1; XM_009449784.2 and XP_009448059.1; XM_001135484.3 and XP_001135484.1), monkey CDH1 (XM_015141010.1 and XP_014996496.1), dog CDH1 (XM_022408269.1 and XP_022263977.1; XM_005617260.3 and XP_005617317.1; XM_022408249.1 and XP_022263957.1; XM_005617262.3 and XP_005617319.1; XM_005617258.3 and XP_005617315.1; XM_022408253.1 and XP_022263961.1; XM_005617257.3 and XP_005617314.1; XM_022408240.1 and XP_022263948.1; XM_005617259.3 and XP_005617316.1; XM_022408259.1 and XP_022263967.1; XM_022408265.1 and XP_022263973.1), cattle CDH1 (NM_001046357.2 and NP_001039822.1), mouse CDH1 (NM_001289591.1 and NP_001276520.1; NM_001289592.1 and NP_001276521.1; NM_028261.1 and NP_082537.1), and rat CDH1 (NM_001109122.1 and NP_001102592.1).

STING agonists have been shown as useful therapies to treat cancer. Agonists of STING well-known in the art and include, for example, MK-1454, STING agonist-1 (MedChem Express Cat No. HY-19711), cyclic dinucleotides (CDNs) such as cyclic di-AMP (c-di-AMP), cyclic-di-GMP (c-di-GMP), cGMP-AMP (2′3′cGAMP or 3′3′cGAMP), or 10-carboxymethyl-9-acridanone (CMA) (Ohkuri et al. (2015) Oncoimmunology 4(4):e999523), rationally designed synthetic CDN derivative molecules (Fu et al. (2015) Sci Transl Med. 7(283):283ra52. doi: 10.1126/scitranslmed.aaa4306), and 5,6-dimethyl-xanthenone-4-acetic acid (DMXAA) (Corrales et al. (2015) Cell Rep. 11(7): 1018-1030). These agonists bind to and activate STING, leading to a potent type I IFN response. On the other hand, targeting the cGAS-STING pathway with small molecule inhibitors would benefit for the treatment of severe debilitating diseases such as inflammatory and autoimmune diseases associated with excessive type I IFNs production due to aberrant DNA sensing and signaling. STING inhibitors are also known and include, for example, CCCP (MedChem Express, Cat No. HY-100941) and 2-bromopalmitate (Tao et al. (2016) IUBMB Life. 68(11):858-870). It is to be noted that the term can further be used to refer to any combination of features described herein regarding STING molecules. For example, any combination of sequence composition, percentage identify, sequence length, domain structure, functional activity, etc. can be used to describe a STING molecule of the present invention.

The term “STING pathway” or “cGAS-STING pathway” refers to a STING-regulated innate immune pathway, which mediates cytosolic DNA-induced signalling events. Cytosolic DNA binds to and activates cGAS, which catalyzes the synthesis of 2′3′-cGAMP from ATP and GTP. 2′3′-cGAMP binds to the ER adaptor STING, which traffics to the ER-Golgi intermediate compartment (ERGIC) and the Golgi apparatus. STING then activates IKK and TBK1. TBK1 phosphorylates STING, which in turn recruits IRF3 for phosphorylation by TBK1. Phosphorylated IRF3 dimerizes and then enters the nucleus, where it functions with NF-kB to turn on the expression of type I interferons and other immunomodulatory molecules. The cGAS-STING pathway not only mediates protective immune defense against infection by a large variety of DNA-containing pathogens but also detects tumor-derived DNA and generates intrinsic antitumor immunity. However, aberrant activation of the cGAS-STING pathway by self DNA can also lead to autoimmune and inflammatory disease.

The term “PARP” refers to poly (ADP-ribose) polymerase. PARP catalyzes the conversion of β-nicotinamide adenine dinucleotide (NAD+) into nicotinamide and poly-ADP-ribose (PAR). Both poly (ADP-ribose) and PARP have been linked to regulation of transcription, cell proliferation, genomic stability, and carcinogenesis (Bouchard et al. (2003) Experimental Hematology, 31(6):446-454; Herceg et al. (2001) Mutation Research/Fundamental and Molecular Mechanisms of Mutagenesis, 477:97-110). Poly(ADP-ribose) polymerase 1 (PARP1) is a key molecule in the repair of DNA single-strand breaks (SSBs) (de Murcia J. et al. (1997) Proc Natl Acad Sci USA 94:7303-7307; Schreiber et al. (2006) Nat Rev Mol Cell Biol 7:517-528; Wang et al. (1997) Genes Dev 11:2347-2358). Knockout of SSB repair by inhibition of PARP1 function induces DNA double-strand breaks (DSBs) that can trigger synthetic lethality in cancer cells with defective homology-directed DSB repair (Bryant et al. (2005) Nature 434:913-917; Farmer et al. (2005) Nature 434:917-921). PARP (e.g., PARP-1 and/or PARP-2) inhibitors are well-known in the art. FDA-approved PARP inhibitors include Olaparib, Rucaparib, and Niraparib. Olaparib is approved as monotherapy for patients with germline BRCA mutated advanced ovarian cancer who have been treated with three or more prior lines of chemotherapy. Rucaparib is approved for previously treated BRCA-mutant ovarian cancer. Niraparib is approved for epithelial ovarian, fallopian tube and primary peritoneal cancer. Other examples of PARP inhibitors include veliparib (ABT-888), iniparib (BSI-201), talazoparib (BMN-673), BGP-15 (N-Gene Research Laboratories, Inc.), INO-1001 (Inotek Pharmaceuticals Inc.), CEP 9722 (Cephalon, Inc.), E7016 (AbbVie, Inc.), BGB-290 (BeiGene, Ltd.), PJ34 (Soriano et al. (2001) Circ. Res. 89(8):684-91; Pacher et a. (2002) Br. J. Pharmacol. 135(6): 1347-1350), 3-aminobenzamide (Trevigen), 4-amino-1,8-naphthalimide (Trevigen), 6(5H)-phenanthridinone (Trevigen), NMS-P118 (Selleck Chemicals), E7449 (Selleck Chemicals), Niraparib (Postel-Vinay et al. (2013) Oncogene 32:5377-5387), Picolinamide (Selleck Chemicals), AG-14361 (Selleck Chemicals), A-966492 (Selleck Chemicals), PJ34 HCl (Selleck Chemicals), UPF 1069 (Selleck Chemicals), AZD2461 (Selleck Chemicals), ME0328 (Selleck Chemicals), G007-LK (Selleck Chemicals), NVP-TNKS656 (Selleck Chemicals), Benzamide (U.S. Pat. Re. 36,397), 4-HQN (R&D systems), DR 2313 (R&D systems), BYK 204165 (R&D systems), BYK 49187 (R&D systems), EB 47 (R&D systems), JW 55 (R&D systems), and NU1025 (Bowman et al. (2001) Br. J. Cancer 84(1):106-12). The mechanism of action is generally related to the ability of PARP inhibitors to bind PARP and decrease its activity.

The term “DNA damage” refers to chemical and/or physical modification of the DNA in a cell, including, but not limited to, methylation, alkylation double-stranded breaks, cross-linking, thymidine dimers caused by ultraviolet light, and oxidative lesions formed by oxygen radical binding to DNA bases. DNA damage initiates a tightly regulated signaling cascade and the orderly recruitment of repair factors to damage sites as described further below. The chromatin substrate for DNA damage repair (DDR), DNA encircling nucleosomes comprised of core histone proteins, can be modulated in multiple ways—incorporation of histone variants, post-translational modification of select histones, repositioning of nucleosomes and generation of DNA repair foci (Polo and Jackson (2011) Genes Dev. 25:409-433). Cells utilize specific factors to detect and repair DNA single-strand breaks (SSB) and two complementary pathways, homologous recombination (HR) and non-homologous end-joining (NHEJ), to address double-strand breaks (DSB) (Polo and Jackson (2011) Genes Dev. 25:409-433). One of the earliest responses to single-strand and double-strand DNA breaks is the activation and recruitment of poly(ADP-ribose) polymerase protein (PARP) family members. Although the PARP family includes 16 proteins, thus far, only PARP1 and PARP2 have been linked to DNA damage responses (Ciccia and Elledge (2010) Mol. Cell 40:179-204). Upon activation, PARP1 catalyzes the NAD⁺-dependent addition of poly(ADP-ribose) (PAR) chains to target proteins including certain histones and PARP1 itself. PARP1 activation and associated PAR synthesis occur within seconds of DNA damage and persist for minutes (Polo and Jackson (2011) Genes Dev. 25:409-433). The rapid and short-lived PARylation at DNA damage sites is thought to promote a more relaxed chromatin structure which facilitates DNA repair (Krishnakumar and Kraus (2010) Mol. Cell 39:8-24). DDR proteins assemble in a coordinated, sequential manner at sites of DNA breaks (Polo and Jackson (2011) Genes Dev. 25:409-433). The initial recruitment phase is rapid, transient and dependent upon PARylation at DNA damage sites (Polo and Jackson (2011) Genes Dev. 25:409-433). A second phase, which also begins within seconds but lasts for hours, includes the sequential phosphorylation and ubiquitylation of multiple DSB repair factors (Polo and Jackson (2011) Genes Dev. 25:409-433). Following the initial recruitment of the MRN (Mre11, RAD50 and Nbs1) complex, HR DSB repair involves ATM localization and phosphorylation of yH2AX and MDC1 (Polo and Jackson (2011) Genes Dev. 25:409-433). ATM-mediated phosphorylation of MDC1 promotes the recruitment of the RNF8 E3 ligase, which targets H2A histones (Polo and Jackson (2011) Genes Dev. 25:409-433). A second E3 ligase, RNF168, interacts with ubiquitylated H2A-type histones in a RNF8-dependent manner and amplifies the local concentration of ubiquitin conjugates (Doil et al. (2009) Cell 136:435-446; Polo and Jackson (2011) Genes Dev. 25:409-433). Of note, RNF8/RNF168 also regulate the retention of the checkpoint mediators, 53BP1 and BRCA 1, at sites of DNA damage. The mechanisms of RNF8/RNF168-modulated recruitment of 53BP1 remain undefined, whereas BRCA 1 localizes to DNA breaks via RAP80, an adaptor protein with ubiquitininteracting motifs (UIM) (Doil et al. (2009) Cell 136:435-446). In some embodiments, “DNA damage repair” or “DDR” refers to one or more of the DNA damage repair processes described above. In other embodiments, “DNA repair” refers to a collection of mechanisms used to repair damage to DNA.

A non-limiting list of exemplary DNA repair mechanisms includes non-homologous end joining (NHEJ), homologous recombination (HR), single-strand break repair, nucleotide excision repair (NER), base excision repair (BER), mismatch excision repair (MER), and other repair mechanisms using DNA polymerases, editing and processing nucleases and DNA repair helicases. Additional DNA repair mechanisms also include using O⁶-methylguanine-DNA methyltransferase (MGMT) to repair O⁶-alkylguanine lesions. Many genes and genetic elements in mammals (e.g., humans) are well known to those of skill in the art and are available in such compiled forms as Wood et al., Human DNA Repair Genes, Science, 291: 1284-1289 (February 2001) and Bulman et al., Locations of DNA Damage Response and Repair Genes in the Mouse and Correlation with Cancer Risk Modifiers, National Radiological Protection Board Report, October 2004 (ISBN 0-85951-544-3). In addition, a mouse DNA repair gene database is publicly available at the UK Health Protection Agency website. Exemplary proteins mediating NHEJ include, but are not limited to, Ligase4, XRCC4, H2AX, DNAPKcs (DNA-PK), Ku70, Ku80, Artemis, Cernunnos/XLF, MRE11, NBS1, and RAD50. Exemplary homologous recombination proteins include RAD51, RAD52, RAD54, XRCC3, RAD51C, BRCA1, BRCA2 (FANCD1), FANCA, FANCB, FANCC, FANCD2, FANCE, FANCF, FANCG, FANCJ (BRIP1/BACH1), FANCL, FANCM, Chk1, Chk2, ATM, and ATR. Exemplary proteins mediating BER include, but are not limited to, ung, smug1, mbd4, tdg, off1, myh, nth1, mpg, ape1, ape2, lig3, xrcc1, adprt, adprtl2 and adprtl3. Exemplary proteins mediating MER include, but are not limited to, msh2, msh3, msh4, msh5, msh6, pms1, pms3, mlh1, mlh3, pms213 and pms214. Exemplary DNA repair helicases include BLM and WRN. Exemplary proteins mediating NER include, but are not limited to, XPA, XPB, XPC, XPD, XPF, XPG, XPV, RAD23B, USP7, RPA, CAK, ERCC1, RFC, LIG1, LIG3, CSA, CSB, PARP1, NEIL1, and APE1. Modulated expression of one or more of these proteins or nucleic acid elements involved in producing the proteins (e.g., nucleic acid coding sequences, splicing sequences, transcription/translation sequences, enhancers, inhibitors, and other regulatory sequences) are contemplated for use in generating DNA repair-deficiency in cells. In some embodiments, the one or more proteins, or nucleic acid elements involved in producing the proteins are mutated to reduce expression and/or activity of the protein/nucleic acid.

As used herein, “homologous recombination” is one type of DNA repair mechanism involving the use of an intact copy of a gene or chromosome as a template for synthesis of new DNA spanning a double strand break. In higher eukaryotes, homologous recombination occurs predominantly in the G2 phase of the cell cycle, when sister chromatids are available as template (Sonoda et al. (2001) Proc. Natl. Acad. Sci. USA, 98:8388-8394; Lee et al. (1997)Mol. Cell. Biol. 17:1425-1433). BRCA1 and BRCA2 act as an integral component of the homologous recombination machinery (HR) (Narod et al. (2004) Nat Rev Cancer 4:665-676; Gudmundsdottir et al. (2006) Oncogene 25:5864-5874). Cells defective in BRCA1 or BRCA2 have a defect in the repair of double-strand breaks (DSB) by the mechanism of homologous recombination (HR) by gene conversion (Farmer et al. (2005) Nature 434:917-921; Narod et al. (2004) Nat Rev Cancer 4:665-676; Gudmundsdottir et al. (2006) Oncogene 25:5864-5874; Helleday et al. (2008) Nat Rev Cancer 8:193-204). Deficiency in either of the breast cancer susceptibility proteins BRCA1 or BRCA2 induces profound cellular sensitivity to the inhibition of poly(ADP-ribose) polymerase (PARP) activity, resulting in cell cycle arrest and apoptosis. It has been reported that the critical role of BRCA1 and BRCA2 in the repair of double-strand breaks by homologous recombination (HR) is the underlying reason for this sensitivity, and the deficiency of RAD51, RAD54, DSS1, RPA1, NBS1, ATR, ATM, CHK1, CHK2, FANCD2, FANCA, or FANCC induces such sensitivity (McCabe et al. (2006) Cancer research 66:8109-8115). It has been proposed that PARP1 inhibition can be a specific therapy for cancers with defects in BRCA1/2 or other HR pathway components (Helleday et al. (2008) Nat Rev Cancer 8:193-204).

As used herein, “non-homologous end-joining” is one type of DNA repair mechanism involving the ligation of DNA termini, typically intermolecular ligation. It includes the joining of DNA ends which exhibit little or no complementarity to each other (and so, typically, each end does not hybridize to the other) and, in any event, is a term well known in the art as is evidenced by its use in many of the papers and patent applications referred to herein, all of which are incorporated herein by reference. Typically, a NHEJ reaction requires a suitable DNA substrate, and suitable components for the reaction of joining the DNA ends to proceed. Suitable DNA substrates are those that, typically, are linear DNA molecules the length of which need only be large enough to accommodate the factors which participate in NHEJ. Conveniently, each DNA fragment to be joined is, independently, at least 50 bp, preferably at least 70 bp, more preferably at least 100 bp but may be bigger. In relation to the observation of a NHEJ reaction, particularly in a screening assay, one or both of the DNA molecules (or DNA ends) to be joined are detectably labeled such as with radiolabelled phosphorus or with fluorescent labels. Although it is convenient to use two separate DNA molecules to be joined in the NHEJ, two ends of the same molecule can be joined such as the ends of a linearized plasmid. NHEJ typically takes place in a eukaryotic cell, such as a vertebrate cell including mammalian cells (although it can also occur in some circumstances in prokaryotes) but, as is described in detail in Baumann & West (1998) Proc. Natl. Acad. Sci USA 95:14066-14070, it can also occur in cell-free extracts, such as those obtained from human cells as therein described. Intermolecular ligation in this cell-free system was found to be accurate and to depend on DNA ligase IV, XRCC4 and DNA-dependent protein kinase (DNA-PK; this is a heterotrimer made up of a catalytic subunit DNA-PKcs (encoded by the XRCC7 gene) and two further subunits which are believed to be involved in DNA binding, namely Ku70 and Ku80 subunits (which are encoded by the XRCC6 and XRCC5 genes, respectively). However, it is possible to get a low level of NHEJ with DNA ligase IV and XRCC4 in the absence of DNA-PK, but a greater extent of NHEJ is obtained when DNA ligase IV and XRCC4 are present with Ku70 and Ku80, and still further NHEJ is achieved when the catalytic subunit of DNA-PK is present.

The term “microhomology-mediated end joining (MMEJ)”, also known as alternative nonhomologous end-joining (Alt-NHEJ), is one of the pathways for repairing double-strand breaks in DNA. MMEJ is distinguished from the other repair mechanisms by its use of 5-25 base pair microhomologous sequences to align the broken strands before joining (McVey et al. (2008) Trends Genet. 24(11):529-38). MMEJ uses a Ku protein and DNA-PK independent repair mechanism, and repair occurs during the S-phase of the cell cycle, as opposed to the G0/G1 and early S-phases in NHEJ and late S to G2-phase for homologous recombinational repair (HRR). MMEJ and HRR share the initial end resection step in repair of double-strand breaks in mammalian cells. Both processes utilize the MRE11 nuclease. MMEJ is an error-prone method of repair and results in deletion mutations in the genetic code, which may initiate the creation of oncogenes that could lead to the development of cancer. Genes that are required for MMEJ include, but are not limited to, FEN1, Ligase III, MRE11, NBS1, PARP1 and XRCC1 (Sharma et al. (2015) Cell Death Dis. 6:e1697).

The term “DNA damage checkpoint” refers to a pause in the cell cycle that is induced in response to DNA damage to ensure that the damage is repaired before cell division resumes. Proteins that accumulate at the damage site typically activate the checkpoint and halt cell growth at the G1/S or G2/M boundaries. Exemplary DNA Damage Checkpoint proteins include, but are not limited to, ATR, Chk1, NBS1, Hus1, Rad1, Mad2, BubR1, ATM, Rad50, Mre11, Mdc1, 53BP1, Rad17, BubR1, ATRIP, Chk2, H2AX, RFC1, RFC2, RFC3, RFC4, RFC5, ATM, BRCA1, Chk1, Chk2, 14-3-3eta, 14-3-3sigma, cdc25A, cdc25c, wee1, ATR, ATRIP, Rad17, RFC2, RFC3, RFC4, RFC5, HUS1, Rad1, Rad9, P53, Rad50, Mrel 1, NBS1, TopBP1, 53BP1, and H2AX.

There is a known and definite correspondence between the amino acid sequence of a particular protein and the nucleotide sequences that can code for the protein, as defined by the genetic code (shown below). Likewise, there is a known and definite correspondence between the nucleotide sequence of a particular nucleic acid and the amino acid sequence encoded by that nucleic acid, as defined by the genetic code.

| GENETIC CODE | |
|---|---|
| Alanine (Ala, A) | GCA, GCC, GCG, GCT |
| Arginine (Arg, R) | AGA, ACG, CGA, CGC, CGG, CGT |
| Asparagine (Asn, N) | AAC, AAT |
| Aspartic acid (Asp, D) | GAC, GAT |
| Cysteine (Cys, C) | TGC, TGT |
| Glutamic acid (Glu, E) | GAA, GAG |
| Glutamine (Gln, Q) | CAA, CAG |
| Glycine (Gly, G) | GGA, GGC, GGG, GGT |
| Histidine (His, H) | CAC, CAT |
| Isoleucine (Ile, I) | ATA, ATC, ATT |
| Leucine (Leu, L) | CTA, CTC, CTG, CTT, TTA, TTG |
| Lysine (Lys, K) | AAA, AAG |
| Methionine (Met, M) | ATG |
| Phenylalanine (Phe, F) | TTC, TTT |
| Proline (Pro, P) | CCA, CCC, CCG, CCT |
| Serine (Ser, S) | AGC, AGT, TCA, TCC, TCG, TCT |
| Threonine (Thr, T) | ACA, ACC, ACG, ACT |
| Tryptophan (Trp, W) | TGG |
| Tyrosine (Tyr, Y) | TAC, TAT |
| Valine (Val, V) | GTA, GTC, GTG, GTT |
| Termination signal (end) | TAA, TAG, TGA |

An important and well-known feature of the genetic code is its redundancy, whereby, for most of the amino acids used to make proteins, more than one coding nucleotide triplet may be employed (illustrated above). Therefore, a number of different nucleotide sequences may code for a given amino acid sequence. Such nucleotide sequences are considered functionally equivalent since they result in the production of the same amino acid sequence in all organisms (although certain organisms may translate some sequences more efficiently than they do others). Moreover, occasionally, a methylated variant of a purine or pyrimidine may be found in a given nucleotide sequence. Such methylations do not affect the coding relationship between the trinucleotide codon and the corresponding amino acid.

In view of the foregoing, the nucleotide sequence of a DNA or RNA encoding a biomarker nucleic acid (or any portion thereof) can be used to derive the polypeptide amino acid sequence, using the genetic code to translate the DNA or RNA into an amino acid sequence. Likewise, for polypeptide amino acid sequences, corresponding nucleotide sequences that can encode the polypeptide can be deduced from the genetic code (which, because of its redundancy, will produce multiple nucleic acid sequences for any given amino acid sequence). Thus, description and/or disclosure herein of a nucleotide sequence which encodes a polypeptide should be considered to also include description and/or disclosure of the amino acid sequence encoded by the nucleotide sequence. Similarly, description and/or disclosure of a polypeptide amino acid sequence herein should be considered to also include description and/or disclosure of all possible nucleotide sequences that can encode the amino acid sequence.

### II. Cancer Vaccine

The present invention provides a cancer vaccine comprising DNA repair-deficient cancer cells, wherein the cancer cells are contacted with a PARP inhibitor to induce DNA breaks. The cancer cells may be derived from a solid or hematological cancer (e.g., ovarian cancer). In certain embodiments, the ovarian cancer cells are high grade serous ovarian cancer cells (HGSOC). In one embodiment, the cancer cells are derived from a subject. In another embodiment, the cancer cells are derived from a cancer cell line. The cancer cells may be from a cancer cell line, such as a cell line selected from the group consisting of PP, 4T1 (ATCC® CRL-2539), EMT-6 (ATCC® CRL-2755), GL261 (NCI-Development Therapeutics Program (DTP)), MC38 (NCI-DTP), Pan02[CP1] (NCI-DTP), CT26 (ATCC® CRL-2638), KLN205 (ATCC® CRL-1453), Lewis Lung (ATCC® CRL-1642), Madison 109, MBT-2, Colon26, CT26 (ATCC® CRL-2638), A20 (ATCC® TIB-208), E.G7-OVA (ATCC® CRL-2113), B16F10 (ATCC® CRL-6475), ClondmanS91 (ATCC-CCL-53.1), and Renca (ATCC® CRL-2947). The cancer cells may have different kinds of genetic mutations. For example, the cancer cells may be derived from an ovarian cancer driven by co-loss of p53 and Brca1 and overexpression of c-Myc. The cancer cells may be derived from the subject who is treated with the cancer vaccine. The cancer cells may also be derived from a different subject who is not treated with the cancer vaccine. The cancer cells may be derived from a cancer that is the same type as the cancer treated with the cancer vaccine. The cancer cells may also be derived from a cancer that is a different type from the cancer treated with the cancer vaccine.

a. Cancer Cell Isolation and Purification

In some embodiments, the cancer cells are derived from a subject. Isolation and purification of tumor cell from various tumor tissues such as surgical tumor tissues, ascites or carcinous hydrothorax is a common process to obtain the purified tumor cells. Cancer cells may be purified from fresh biopsy samples from cancer patients or animal tumor models. The biopsy samples often contain a heterogeneous population of cells that include normal tissue, blood, and cancer cells. Preferably, a purified cancer cell composition can have greater than 70%, 75%, 80%, 85%, 90%, 95%, 99%, or more, or any range in between or any value in between, total viable cancer cells. To purify cancer cells from the heterogeneous population, a number of methods can be used.

In one embodiment, laser microdissection is used to isolate cancer cells. Cancer cells of interest can be carefully dissected from thin tissue slices prepared for microscopy. In this method, the tissue section is coated with a thin plastic film and an area containing the selected cells is irradiated with a focused infrared laser beam pulse. This melts a small circle in the plastic film, causing cell binding underneath. Those captured cells are removed for additional analysis. This technique is good for separating and analyzing cells from different parts of a tumor, which allows for a comparison of their similar and distinct properties. It was used recently to analyze pituitary cells from dissociated tissues and from cultured populations of heterogeneous pituitary, thyroid, and carcinoid tumor cells, as well as analyzing single cells found in various sarcomas.

In another embodiment, fluorescence activated cell sorting (FACS), also referred to as flow cytometry, is used to sort and analyze the different cell populations. Cells having a cellular marker or other specific marker of interest are tagged with an antibody, or typically a mixture of antibodies, that bind the cellular markers. Each antibody directed to a different marker is conjugated to a detectable molecule, particularly a fluorescent dye that may be distinguished from other fluorescent dyes coupled to other antibodies. A stream of tagged or “stained” cells is passed through a light source that excites the fluorochrome and the emission spectrum from the cells detected to determine the presence of a particular labeled antibody. By concurrent detection of different fluorochromes, also referred to in the art as multicolor fluorescence cell sorting, cells displaying different sets of cell markers may be identified and isolated from other cells in the population. Other FACS parameters, including, by way of example and not limitation, side scatter (SSC), forward scatter (FSC), and vital dye staining (e.g., with propidium iodide) allow selection of cells based on size and viability. FACS sorting and analysis of HSC and related lineage cells is well-known in the art and described in, for example, U.S. Pat. Nos. 5,137,809; 5,750,397; 5,840,580; 6,465,249; Manz et al. (202) Proc. Natl. Acad. Sci. U.S.A. 99:11872-11877; and Akashi et al. (200) Nature 404:193-197. General guidance on fluorescence activated cell sorting is described in, for example, Shapiro (2003) Practical Flow Cytometry, 4th Ed., Wiley-Liss (2003) and Ormerod (2000) Flow Cytometry: A Practical Approach, 3rd Ed., Oxford University Press.

Another method of isolating useful cell populations involves a solid or insoluble substrate to which is bound antibodies or ligands that interact with specific cell surface markers. In immunoadsorption techniques, cells are contacted with the substrate (e.g., column of beads, flasks, magnetic particles, etc.) containing the antibodies and any unbound cells removed. Immunoadsorption techniques may be scaled up to deal directly with the large numbers of cells in a clinical harvest. Suitable substrates include, by way of example and not limitation, plastic, cellulose, dextran, polyacrylamide, agarose, and others known in the art (e.g., Pharmacia Sepharose 6 MB macrobeads). When a solid substrate comprising magnetic or paramagnetic beads is used, cells bound to the beads may be readily isolated by a magnetic separator (see, e.g., Kato and Radbruch (1993) Cytometry 14:384-92). Affinity chromatographic cell separations typically involve passing a suspension of cells over a support bearing a selective ligand immobilized to its surface. The ligand interacts with its specific target molecule on the cell and is captured on the matrix. The bound cell is released by the addition of an elution agent to the running buffer of the column and the free cell is washed through the column and harvested as a homogeneous population. As apparent to the skilled artisan, adsorption techniques are not limited to those employing specific antibodies, and may use nonspecific adsorption. For example, adsorption to silica is a simple procedure for removing phagocytes from cell preparations. One of the most common uses of this technology is for isolating circulating tumor cells (CTCs) from the blood of breast, NSC lung cancer, prostate and colon cancer patients using an antibody against EpCAM, a cell surface glycoprotein that has been found to be highly expressed in epithelial cancers.

FACS and most batch wise immunoadsorption techniques may be adapted to both positive and negative selection procedures (see, e.g., U.S. Pat. No. 5,877,299). In positive selection, the desired cells are labeled with antibodies and removed away from the remaining unlabeled/unwanted cells. In negative selection, the unwanted cells are labeled and removed. Another type of negative selection that may be employed is use of antibody/complement treatment or immunotoxins to remove unwanted cells.

In still another embodiment, microfluidics, one of the newest technologies, is used to isolate cancer cells. This method used a microfluidic chip with a spiral channel that can isolate circulating tumor cells (CTCs) from blood based upon their size. A sample of blood is pumped into the device and as cells flow through the channel at high speeds, the inertial and centrifugal forces cause smaller cells to flow along the outer wall while larger cells, including CTCs, flow along the inner wall. Researchers have used this chip technology to isolate CTCs from the blood of patients with metastatic lung or breast cancer.

Fluorescent nanodiamonds (FNDs), according to a recently published article (Lin et al. Small (2015) 11:4394-4402), can be used to label and isolate slow-proliferating/quiescent cancer stem cells, which, according to study authors, have been difficult to isolate and track over extended time periods using traditional fluorescent markers. It was concluded that nanoparticles do not cause DNA damage or impair cell growth, and that they outperformed EdU and CFSE fluorescent labels in terms of long-term tracking capability.

It is to be understood that the purification or isolation of cells also includes combinations of the methods described above. A typical combination may comprise an initial procedure that is effective in removing the bulk of unwanted cells and cellular material. A second step may include isolation of cells expressing a marker common to one or more of the progenitor cell populations by immunoadsorption on antibodies bound to a substrate. An additional step providing higher resolution of different cell types, such as FACS sorting with antibodies to a set of specific cellular markers, may be used to obtain substantially pure populations of the desired cells.

b. Cancer Cell Engineering and Modification

The cancer cells comprised in the cancer vaccine are DNA repair-deficient. In some embodiments, cancer cells are DNA repair-deficient due to genetic mutations acquired by the cancer cells during cancer transformation or progression. In some other embodiments, cancer cells are engineered to become DNA repair-deficient with an agent that reduces copy number, amount, and/or activity of one or more DNA damage checkpoints and/or DNA damage repair genes. The one or more DNA damage checkpoint genes may be selected from the group consisting of Brca1, Brca2, Chk1, Chk2, ATM, ATR, Cdc25C, and Nbs1. The one or more DNA damage repair genes may be involved in the non-homologous end joining (NHEJ), microhomology-mediated end joining (MMEJ), or homologous recombination pathway. For example, the one or more DNA damage repair genes may be selected from the group consisting of BLM, MSH2, MSH6, MLH1, PMS2, MRE11, DNA Ligase IV, TP53BP1, RAD51, RAD51L1, RAD51C, RAD51L3, DMC1, XRCC2, XRCC3, XRCC4, NBS1, RAD50, GADD45, RFC2, XRCC6, POLD2, PCNA, RPA1, RPA2, ERCC3, UNG, ERCC5, MLH1, LIG1, NBN, MSH6, POLD4, RFC5, DDB2, POLD1, FANCG, POLB, XRCC1, MPG, RFC2, ERCC1, TDG, FANCA, RFC4, RFC3, APEX2, RAD1, BRCA1, FEN1, MLH3, MGMT, RAD51, XRCC4, RECQL, ERCC8, FANCC, OGG1, MRE11A, RAD52, WRN, XPA, BLM, OGG1, MSH3, POLE2, RAD51C, LIG4, ERCC6, LIG3, RAD17, XRCC2, MUTYH, RFC1, BRCA2, RAD50, DDB1, XRCC5, PARP1, POLE3, RFC1, RAD50, XPC, MSH2, RPA3, MBD4, NTHL1, PMS2/PMS2CL, RAD51C, UNG2, APEX1, ERCC4, RAD1, RECQL5, MSH5, RECQL, RAD52, XRCC4, RAD17, MSH3, MRE11A, MSH6, and RECQL5.

The agent that reduces copy number, amount, and/or activity of one or more DNA damage checkpoints and/or DNA damage repair genes could be a small molecule inhibitor, CRISPR guide RNA (gRNA), RNA interfering agent, antisense oligonucleotide, peptide or peptidomimetic inhibitor, aptamer, antibody, or intrabody.

In one embodiment, peptides or peptide mimetics can be used to antagonize the activity of one or more DNA damage checkpoints and/or DNA damage repair genes. In one embodiment, variants of one or more DNA damage checkpoints and/or DNA damage repair genes which function as a modulating agent for the respective full length protein, can be identified by screening combinatorial libraries of mutants, e.g., truncation mutants, for antagonist activity. In one embodiment, a variegated library of variants is generated by combinatorial mutagenesis at the nucleic acid level and is encoded by a variegated gene library. A variegated library of variants can be produced, for instance, by enzymatically ligating a mixture of synthetic oligonucleotides into gene sequences such that a degenerate set of potential polypeptide sequences is expressible as individual polypeptides containing the set of polypeptide sequences therein. There are a variety of methods which can be used to produce libraries of polypeptide variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential polypeptide sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477.

In addition, libraries of fragments of a polypeptide coding sequence can be used to generate a variegated population of polypeptide fragments for screening and subsequent selection of variants of a given polypeptide. In one embodiment, a library of coding sequence fragments can be generated by treating a double stranded PCR fragment of a polypeptide coding sequence with a nuclease under conditions wherein nicking occurs only about once per polypeptide, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the polypeptide.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of polypeptides. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of interest (Arkin and Youvan (1992) Proc. Natl. Acad. Sci. USA 89:7811-7815; Delagrave et al. (1993) Protein Eng. 6(3):327-331). In one embodiment, cell based assays can be exploited to analyze a variegated polypeptide library. For example, a library of expression vectors can be transfected into a cell line which ordinarily synthesizes one or more DNA damage checkpoints and/or DNA damage repair genes. The transfected cells are then cultured such that the full length polypeptide and a particular mutant polypeptide are produced and the effect of expression of the mutant on the full length polypeptide activity in cell supernatants can be detected, e.g., by any of a number of functional assays. Plasmid DNA can then be recovered from the cells which score for inhibition, or alternatively, potentiation of full length polypeptide activity, and the individual clones further characterized.

Systematic substitution of one or more amino acids of a polypeptide amino acid sequence with a D-amino acid of the same type (e.g., D-lysine in place of L-lysine) can be used to generate more stable peptides. In addition, constrained peptides comprising a polypeptide amino acid sequence of interest or a substantially identical sequence variation can be generated by methods known in the art (Rizo and Gierasch (1992) Annu. Rev. Biochem. 61:387, incorporated herein by reference); for example, by adding internal cysteine residues capable of forming intramolecular disulfide bridges which cyclize the peptide.

The amino acid sequences disclosed herein will enable those of skill in the art to produce polypeptides corresponding peptide sequences and sequence variants thereof. Such polypeptides can be produced in prokaryotic or eukaryotic host cells by expression of polynucleotides encoding the peptide sequence, frequently as part of a larger polypeptide. Alternatively, such peptides can be synthesized by chemical methods. Methods for expression of heterologous proteins in recombinant hosts, chemical synthesis of polypeptides, and in vitro translation are well-known in the art and are described further in Maniatis et al. Molecular Cloning: A Laboratory Manual (1989), 2nd Ed., Cold Spring Harbor, N.Y.; Berger and Kimmel, Methods in Enzymology, Volume 152, Guide to Molecular Cloning Techniques (1987), Academic Press, Inc., San Diego, Calif.; Merrifield, J. (1969) J. Am. Chem. Soc. 91:501; Chaiken I. M. (1981) CRC Crit. Rev. Biochem. 11: 255; Kaiser et al. (1989) Science 243:187; Merrifield, B. (1986) Science 232:342; Kent, S. B. H. (1988) Annu. Rev. Biochem. 57:957; and Offord, R. E. (1980) Semisynthetic Proteins, Wiley Publishing, which are incorporated herein by reference).

Peptides can be produced, typically by direct chemical synthesis. Peptides can be produced as modified peptides, with nonpeptide moieties attached by covalent linkage to the N-terminus and/or C-terminus. In certain preferred embodiments, either the carboxy-terminus or the amino-terminus, or both, are chemically modified. The most common modifications of the terminal amino and carboxyl groups are acetylation and amidation, respectively. Amino-terminal modifications such as acylation (e.g., acetylation) or alkylation (e.g., methylation) and carboxy-terminal-modifications such as amidation, as well as other terminal modifications, including cyclization, can be incorporated into various embodiments of the invention. Certain amino-terminal and/or carboxy-terminal modifications and/or peptide extensions to the core sequence can provide advantageous physical, chemical, biochemical, and pharmacological properties, such as: enhanced stability, increased potency and/or efficacy, resistance to serum proteases, desirable pharmacokinetic properties, and others. Peptides disclosed herein can be used therapeutically to treat disease, e.g., by altering costimulation in a patient.

Peptidomimetics (Fauchere (1986) Adv. Drug Res. 15:29; Veber and Freidinger (1985) TINS p. 392; and Evans et al. (1987) J. Med. Chem. 30:1229, which are incorporated herein by reference) are usually developed with the aid of computerized molecular modeling. Peptide mimetics that are structurally similar to therapeutically useful peptides can be used to produce an equivalent therapeutic or prophylactic effect. Generally, peptidomimetics are structurally similar to a paradigm polypeptide (i.e., a polypeptide that has a biological or pharmacological activity), but have one or more peptide linkages optionally replaced by a linkage selected from the group consisting of: —CH2NH—, —CH2S—, —CH2-CH2-, —CH═CH— (cis and trans), —COCH2-, —CH(OH)CH2-, and —CH2SO—, by methods known in the art and further described in the following references: Spatola, A. F. in “Chemistry and Biochemistry of Amino Acids, Peptides, and Proteins” Weinstein, B., ed., Marcel Dekker, New York, p. 267 (1983); Spatola, A. F., (1983) Vega Data Vol. 1, Issue 3, “Peptide Backbone Modifications” (general review); Morley, J. S. (1980) Trends Pharm. Sci. 463-468 (general review); Hudson, D. et al. (1979) Int. J. Pept. Prot. Res. 14:177-185 (—CH2NH—, CH2CH2-); Spatola, A. F. et al. (1986) Life Sci. 38:1243-1249 (—CH2-S); Hann, M. M. (1982)J. Chem. Soc. Perkin Trans. I. 307-314 (—CH—CH—, cis and trans); Almquist, R. G. et al. (1980)J. Med. Chem. 23:1392-1398 (—COCH2-); Jennings-White, C. et al. (1982) Tetrahedron Lett. 23:2533 (—COCH2-); Szelke, M. et al. (1982) European Appln. EP 45665 CA: 97:39405 (—CH(OH)CH2-); Holladay, M. W. et al. (1983) Tetrahedron Lett. 24:4401-4404 (—C(OH)CH2-); and Hruby, V. J. (1982) Life Sci. 31:189-199 (—CH2-S—); each of which is incorporated herein by reference. A particularly preferred non-peptide linkage is —CH2NH—. Such peptide mimetics may have significant advantages over polypeptide embodiments, including, for example: more economical production, greater chemical stability, enhanced pharmacological properties (half-life, absorption, potency, efficacy, etc.), altered specificity (e.g., a broad-spectrum of biological activities), reduced antigenicity, and others. Labeling of peptidomimetics usually involves covalent attachment of one or more labels, directly or through a spacer (e.g., an amide group), to non-interfering position(s) on the peptidomimetic that are predicted by quantitative structure-activity data and/or molecular modeling. Such non-interfering positions generally are positions that do not form direct contacts with the macropolypeptides(s) to which the peptidomimetic binds to produce the therapeutic effect. Derivatization (e.g., labeling) of peptidomimetics should not substantially interfere with the desired biological or pharmacological activity of the peptidomimetic.

Also encompassed by the present invention are small molecules which can modulate (either enhance or inhibit) activity of one or more DNA damage checkpoints and/or DNA damage repair genes or their interactions with their natural binding partners. The small molecules of the present invention can be obtained using any of the numerous approaches in combinatorial library methods known in the art, including: spatially addressable parallel solid phase or solution phase libraries; synthetic library methods requiring deconvolution; the ‘one-bead one-compound’ library method; and synthetic library methods using affinity chromatography selection. (Lam, K. S. (1997) Anticancer Drug Des. 12:145).

Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of compounds can be presented in solution (e.g., Houghten (1992) Biotechniques 13:412-421), or on beads (Lam (1991) Nature 354:82-84), chips (Fodor (1993) Nature 364:555-556), bacteria (Ladner U.S. Pat. No. 5,223,409), spores (Ladner USP '409), plasmids (Cull et al. (1992) Proc. Natl. Acad. Sci. USA 89:1865-1869) or on phage (Scott and Smith (1990) Science 249:386-390); (Devlin (1990) Science 249:404-406); (Cwirla et al. (1990) Proc. Natl. Acad. Sci. USA 87:6378-6382); (Felici (1991) J. Mol. Biol. 222:301-310); (Ladner supra.). Compounds can be screened in cell based or non-cell based assays. Compounds can be screened in pools (e.g. multiple compounds in each testing sample) or as individual compounds.

Also provided herein are compositions comprising one or more nucleic acids comprising or capable of expressing at least 1, 2, 3, 4, 5, 10, 20 or more small nucleic acids or antisense oligonucleotides or derivatives thereof, wherein said small nucleic acids or antisense oligonucleotides or derivatives thereof in a cell specifically hybridize (e.g., bind) under cellular conditions, with cellular nucleic acids (e.g., small non-coding RNAS such as miRNAs, pre-miRNAs, pri-miRNAs, miRNA*, anti-miRNA, a miRNA binding site, a variant and/or functional variant thereof, cellular mRNAs or a fragments thereof). In one embodiment, expression of the small nucleic acids or antisense oligonucleotides or derivatives thereof in a cell can inhibit expression or biological activity of cellular nucleic acids and/or proteins, e.g., by inhibiting transcription, translation and/or small nucleic acid processing of, for example, one or more DNA damage checkpoints and/or DNA damage repair genes. In one embodiment, the small nucleic acids or antisense oligonucleotides or derivatives thereof are small RNAs (e.g., microRNAs) or complements of small RNAs. In another embodiment, the small nucleic acids or antisense oligonucleotides or derivatives thereof can be single or double stranded and are at least six nucleotides in length and are less than about 1000, 900, 800, 700, 600, 500, 400, 300, 200, 100, 50, 40, 30, 25, 24, 23, 22, 21, 20, 19, 18, 17, 16, 15, or 10 nucleotides in length. In another embodiment, a composition may comprise a library of nucleic acids comprising or capable of expressing small nucleic acids or antisense oligonucleotides or derivatives thereof, or pools of said small nucleic acids or antisense oligonucleotides or derivatives thereof. A pool of nucleic acids may comprise about 2-5, 5-10, 10-20, 10-30 or more nucleic acids comprising or capable of expressing small nucleic acids or antisense oligonucleotides or derivatives thereof.

In one embodiment, binding may be by conventional base pair complementarity, or, for example, in the case of binding to DNA duplexes, through specific interactions in the major groove of the double helix. In general, “antisense” refers to the range of techniques generally employed in the art, and includes any process that relies on specific binding to oligonucleotide sequences.

It is well-known in the art that modifications can be made to the sequence of a miRNA or a pre-miRNA without disrupting miRNA activity. As used herein, the term “functional variant” of a miRNA sequence refers to an oligonucleotide sequence that varies from the natural miRNA sequence, but retains one or more functional characteristics of the miRNA (e.g. cancer cell proliferation inhibition, induction of cancer cell apoptosis, enhancement of cancer cell susceptibility to chemotherapeutic agents, specific miRNA target inhibition). In some embodiments, a functional variant of a miRNA sequence retains all of the functional characteristics of the miRNA. In certain embodiments, a functional variant of a miRNA has a nucleobase sequence that is a least about 60%, 65%, 70%, 75%, 80%, 85%, 90%, 91%, 92%, 93%, 94%, 95%, 96%, 97%, 98% or 99% identical to the miRNA or precursor thereof over a region of about 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleobases, or that the functional variant hybridizes to the complement of the miRNA or precursor thereof under stringent hybridization conditions. Accordingly, in certain embodiments the nucleobase sequence of a functional variant is capable of hybridizing to one or more target sequences of the miRNA.

MicroRNAs and their corresponding stem-loop sequences described herein may be found in miRBase, an online searchable database of miRNA sequences and annotation, found on the world wide web at microrna.sanger.ac.uk. Entries in the miRBase Sequence database represent a predicted hairpin portion of a miRNA transcript (the stem-loop), with information on the location and sequence of the mature miRNA sequence. The miRNA stem-loop sequences in the database are not strictly precursor miRNAs (pre-miRNAs), and may in some instances include the pre-miRNA and some flanking sequence from the presumed primary transcript. The miRNA nucleobase sequences described herein encompass any version of the miRNA, including the sequences described in Release 10.0 of the miRBase sequence database and sequences described in any earlier Release of the miRBase sequence database. A sequence database release may result in the re-naming of certain miRNAs. A sequence database release may result in a variation of a mature miRNA sequence.

In some embodiments, miRNA sequences of the invention may be associated with a second RNA sequence that may be located on the same RNA molecule or on a separate RNA molecule as the miRNA sequence. In such cases, the miRNA sequence may be referred to as the active strand, while the second RNA sequence, which is at least partially complementary to the miRNA sequence, may be referred to as the complementary strand. The active and complementary strands are hybridized to create a double-stranded RNA that is similar to a naturally occurring miRNA precursor. The activity of a miRNA may be optimized by maximizing uptake of the active strand and minimizing uptake of the complementary strand by the miRNA protein complex that regulates gene translation. This can be done through modification and/or design of the complementary strand.

In some embodiments, the complementary strand is modified so that a chemical group other than a phosphate or hydroxyl at its 5′ terminus. The presence of the 5′ modification apparently eliminates uptake of the complementary strand and subsequently favors uptake of the active strand by the miRNA protein complex. The 5′ modification can be any of a variety of molecules known in the art, including NH₂, NHCOCH₃, and biotin.

In another embodiment, the uptake of the complementary strand by the miRNA pathway is reduced by incorporating nucleotides with sugar modifications in the first 2-6 nucleotides of the complementary strand. It should be noted that such sugar modifications can be combined with the 5′ terminal modifications described above to further enhance miRNA activities.

In some embodiments, the complementary strand is designed so that nucleotides in the 3′ end of the complementary strand are not complementary to the active strand. This results in double-strand hybrid RNAs that are stable at the 3′ end of the active strand but relatively unstable at the 5′ end of the active strand. This difference in stability enhances the uptake of the active strand by the miRNA pathway, while reducing uptake of the complementary strand, thereby enhancing miRNA activity.

Small nucleic acid and/or antisense constructs of the methods and compositions presented herein can be delivered, for example, as an expression plasmid which, when transcribed in the cell, produces RNA which is complementary to at least a unique portion of cellular nucleic acids (e.g., small RNAs, mRNA, and/or genomic DNA). Alternatively, the small nucleic acid molecules can produce RNA which encodes mRNA, miRNA, pre-miRNA, pri-miRNA, miRNA*, anti-miRNA, or a miRNA binding site, or a variant thereof. For example, selection of plasmids suitable for expressing the miRNAs, methods for inserting nucleic acid sequences into the plasmid, and methods of delivering the recombinant plasmid to the cells of interest are within the skill in the art. See, for example, Zeng et al. (2002) Mol. Cell 9:1327-1333; Tuschl (2002), Nat. Biotechnol. 20:446-448; Brummelkamp et al. (2002) Science 296:550-553; Miyagishi et al. (2002) Nat. Biotechnol. 20:497-500; Paddison et al. (2002) Genes Dev. 16:948-958; Lee et al. (2002) Nat. Biotechnol. 20:500-505; and Paul et al. (2002) Nat. Biotechnol. 20:505-508, the entire disclosures of which are herein incorporated by reference.

Alternatively, small nucleic acids and/or antisense constructs are oligonucleotide probes that are generated ex vivo and which, when introduced into the cell, results in hybridization with cellular nucleic acids. Such oligonucleotide probes are preferably modified oligonucleotides that are resistant to endogenous nucleases, e.g., exonucleases and/or endonucleases, and are therefore stable in vivo. Exemplary nucleic acid molecules for use as small nucleic acids and/or antisense oligonucleotides are phosphoramidate, phosphothioate and methylphosphonate analogs of DNA (see also U.S. Pat. Nos. 5,176,996; 5,264,564; and 5,256,775). Additionally, general approaches to constructing oligomers useful in antisense therapy have been reviewed, for example, by Van der Krol et al. (1988) BioTechniques 6:958-976; and Stein et al. (1988) Cancer Res 48:2659-2668.

Antisense approaches may involve the design of oligonucleotides (either DNA or RNA) that are complementary to cellular nucleic acids (e.g., complementary to one or more DNA damage checkpoints and/or DNA damage repair genes). Absolute complementarity is not required. In the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with a nucleic acid (e.g., RNA) it may contain and still form a stable duplex (or triplex, as the case may be). One skilled in the art can ascertain a tolerable degree of mismatch by use of standard procedures to determine the melting point of the hybridized complex.

Oligonucleotides that are complementary to the 5′ end of the mRNA, e.g., the 5′ untranslated sequence up to and including the AUG initiation codon, should work most efficiently at inhibiting translation. However, sequences complementary to the 3′ untranslated sequences of mRNAs have recently been shown to be effective at inhibiting translation of mRNAs as well (Wagner (1994) Nature 372:333). Therefore, oligonucleotides complementary to either the 5′ or 3′ untranslated, non-coding regions of genes could be used in an antisense approach to inhibit translation of endogenous mRNAs. Oligonucleotides complementary to the 5′ untranslated region of the mRNA may include the complement of the AUG start codon. Antisense oligonucleotides complementary to mRNA coding regions are less efficient inhibitors of translation but could also be used in accordance with the methods and compositions presented herein. Whether designed to hybridize to the 5′, 3′ or coding region of cellular mRNAs, small nucleic acids and/or antisense nucleic acids should be at least six nucleotides in length, and can be less than about 1000, 900, 800, 700, 600, 500, 400, 300, 200, 100, 50, 40, 30, 25, 24, 23, 22, 21, 20, 19, 18, 17, 16, 15, or 10 nucleotides in length.

Regardless of the choice of target sequence, it is preferred that in vitro studies are first performed to quantitate the ability of the antisense oligonucleotide to inhibit gene expression. In one embodiment these studies utilize controls that distinguish between antisense gene inhibition and nonspecific biological effects of oligonucleotides. In another embodiment these studies compare levels of the target nucleic acid or protein with that of an internal control nucleic acid or protein. Additionally, it is envisioned that results obtained using the antisense oligonucleotide are compared with those obtained using a control oligonucleotide. It is preferred that the control oligonucleotide is of approximately the same length as the test oligonucleotide and that the nucleotide sequence of the oligonucleotide differs from the antisense sequence no more than is necessary to prevent specific hybridization to the target sequence.

Small nucleic acids and/or antisense oligonucleotides can be DNA or RNA or chimeric mixtures or derivatives or modified versions thereof, single-stranded or double-stranded. Small nucleic acids and/or antisense oligonucleotides can be modified at the base moiety, sugar moiety, or phosphate backbone, for example, to improve stability of the molecule, hybridization, etc., and may include other appended groups such as peptides (e.g., for targeting host cell receptors), or agents facilitating transport across the cell membrane (see, e.g., Letsinger et al. (1989) Proc. Natl. Acad. Sci. U.S.A. 86:6553-6556; Lemaitre et al. (1987) Proc. Natl. Acad. Sci. U.S.A. 84:648-652; PCT Publication No. W088/09810) or the blood-brain barrier (see, e.g., PCT Publication No. W089/10134), hybridization-triggered cleavage agents. (See, e.g., Krol et al. (1988) BioTech. 6:958-976) or intercalating agents. (See, e.g., Zon (1988) Pharm. Res. 5:539-549). To this end, small nucleic acids and/or antisense oligonucleotides may be conjugated to another molecule, e.g., a peptide, hybridization triggered cross-linking agent, transport agent, hybridization-triggered cleavage agent, etc.

Small nucleic acids and/or antisense oligonucleotides may comprise at least one modified base moiety which is selected from the group including but not limited to 5-fluorouracil, 5-bromouracil, 5-chlorouracil, 5-iodouracil, hypoxanthine, xantine, 4-acetylcytosine, 5-(carboxyhydroxytiethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Small nucleic acids and/or antisense oligonucleotides may also comprise at least one modified sugar moiety selected from the group including but not limited to arabinose, 2-fluoroarabinose, xylulose, and hexose.

In certain embodiments, a compound comprises an oligonucleotide (e.g., a miRNA or miRNA encoding oligonucleotide) conjugated to one or more moieties which enhance the activity, cellular distribution or cellular uptake of the resulting oligonucleotide. In certain such embodiments, the moiety is a cholesterol moiety (e.g., antagomirs) or a lipid moiety or liposome conjugate. Additional moieties for conjugation include carbohydrates, phospholipids, biotin, phenazine, folate, phenanthridine, anthraquinone, acridine, fluoresceins, rhodamines, coumarins, and dyes. In certain embodiments, a conjugate group is attached directly to the oligonucleotide. In certain embodiments, a conjugate group is attached to the oligonucleotide by a linking moiety selected from amino, hydroxyl, carboxylic acid, thiol, unsaturations (e.g., double or triple bonds), 8-amino-3,6-dioxaoctanoic acid (ADO), succinimidyl 4-(N-maleimidomethyl) cyclohexane-1-carboxylate (SMCC), 6-aminohexanoic acid (AHEX or AHA), substituted C1-C10 alkyl, substituted or unsubstituted C2-C10 alkenyl, and substituted or unsubstituted C2-C10 alkynyl. In certain such embodiments, a substituent group is selected from hydroxyl, amino, alkoxy, carboxy, benzyl, phenyl, nitro, thiol, thioalkoxy, halogen, alkyl, aryl, alkenyl and alkynyl.

In certain such embodiments, the compound comprises the oligonucleotide having one or more stabilizing groups that are attached to one or both termini of the oligonucleotide to enhance properties such as, for example, nuclease stability. Included in stabilizing groups are cap structures. These terminal modifications protect the oligonucleotide from exonuclease degradation, and can help in delivery and/or localization within a cell. The cap can be present at the 5′-terminus (5′-cap), or at the 3′-terminus (3′-cap), or can be present on both termini. Cap structures include, for example, inverted deoxy abasic caps.

Suitable cap structures include a 4′,5′-methylene nucleotide, a 1-(beta-D-erythrofuranosyl) nucleotide, a 4′-thio nucleotide, a carbocyclic nucleotide, a 1,5-anhydrohexitol nucleotide, an L-nucleotide, an alpha-nucleotide, a modified base nucleotide, a phosphorodithioate linkage, a threo-pentofuranosyl nucleotide, an acyclic 3′,4′-seco nucleotide, an acyclic 3,4-dihydroxybutyl nucleotide, an acyclic 3,5-dihydroxypentyl nucleotide, a 3′-3′-inverted nucleotide moiety, a 3′-3′-inverted abasic moiety, a 3′-2′-inverted nucleotide moiety, a 3′-2′-inverted abasic moiety, a 1,4-butanediol phosphate, a 3′-phosphoramidate, a hexylphosphate, an aminohexyl phosphate, a 3′-phosphate, a 3′-phosphorothioate, a phosphorodithioate, a bridging methylphosphonate moiety, and a non-bridging methylphosphonate moiety 5′-amino-alkyl phosphate, a 1,3-diamino-2-propyl phosphate, 3-aminopropyl phosphate, a 6-aminohexyl phosphate, a 1,2-aminododecyl phosphate, a hydroxypropyl phosphate, a 5′-5′-inverted nucleotide moiety, a 5′-5′-inverted abasic moiety, a 5′-phosphoramidate, a 5′-phosphorothioate, a 5′-amino, a bridging and/or non-bridging 5′-phosphoramidate, a phosphorothioate, and a 5′-mercapto moiety.

Small nucleic acids and/or antisense oligonucleotides can also contain a neutral peptide-like backbone. Such molecules are termed peptide nucleic acid (PNA)-oligomers and are described, e.g., in Perry-O'Keefe et al. (1996) Proc. Natl. Acad. Sci. U.S.A. 93:14670 and in Eglom et al. (1993) Nature 365:566. One advantage of PNA oligomers is their capability to bind to complementary DNA essentially independently from the ionic strength of the medium due to the neutral backbone of the DNA. In yet another embodiment, small nucleic acids and/or antisense oligonucleotides comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In a further embodiment, small nucleic acids and/or antisense oligonucleotides are α-anomeric oligonucleotides. An α-anomeric oligonucleotide forms specific double-stranded hybrids with complementary RNA in which, contrary to the usual b-units, the strands run parallel to each other (Gautier et al. (1987) Nucl. Acids Res. 15:6625-6641). The oligonucleotide is a 2′-O-methylribonucleotide (Inoue et al. (1987) Nucl. Acids Res. 15:6131-6148), or a chimeric RNA-DNA analogue (Inoue et al. (1987) FEBS Lett. 215:327-330).

Small nucleic acids and/or antisense oligonucleotides of the methods and compositions presented herein may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. (1988) Nucl. Acids Res. 16:3209, methylphosphonate oligonucleotides can be prepared by use of controlled pore glass polymer supports (Sarin et al. (1988) Proc. Natl. Acad. Sci. U.S.A. 85:7448-7451), etc. For example, an isolated miRNA can be chemically synthesized or recombinantly produced using methods known in the art. In some instances, miRNA are chemically synthesized using appropriately protected ribonucleoside phosphoramidites and a conventional DNA/RNA synthesizer. Commercial suppliers of synthetic RNA molecules or synthesis reagents include, e.g., Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), Cruachem (Glasgow, UK), and Exiqon (Vedbaek, Denmark).

Small nucleic acids and/or antisense oligonucleotides can be delivered to cells in vivo. A number of methods have been developed for delivering small nucleic acids and/or antisense oligonucleotides DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systematically.

In one embodiment, small nucleic acids and/or antisense oligonucleotides may comprise or be generated from double stranded small interfering RNAs (siRNAs), in which sequences fully complementary to cellular nucleic acids (e.g. mRNAs) sequences mediate degradation or in which sequences incompletely complementary to cellular nucleic acids (e.g., mRNAs) mediate translational repression when expressed within cells. In another embodiment, double stranded siRNAs can be processed into single stranded antisense RNAs that bind single stranded cellular RNAs (e.g., microRNAs) and inhibit their expression. RNA interference (RNAi) is the process of sequence-specific, post-transcriptional gene silencing in animals and plants, initiated by double-stranded RNA (dsRNA) that is homologous in sequence to the silenced gene. In vivo, long dsRNA is cleaved by ribonuclease III to generate 21- and 22-nucleotide siRNAs. It has been shown that 21-nucleotide siRNA duplexes specifically suppress expression of endogenous and heterologous genes in different mammalian cell lines, including human embryonic kidney (293) and HeLa cells (Elbashir et al. (2001) Nature 411:494-498). Accordingly, translation of a gene in a cell can be inhibited by contacting the cell with short double stranded RNAs having a length of about 15 to 30 nucleotides or of about 18 to 21 nucleotides or of about 19 to 21 nucleotides. Alternatively, a vector encoding for such siRNAs or short hairpin RNAs (shRNAs) that are metabolized into siRNAs can be introduced into a target cell (see, e.g., McManus et al. (2002) RNA 8:842; Xia et al. (2002) Nature Biotechnology 20:1006; and Brummelkamp et al. (2002) Science 296:550). Vectors that can be used are commercially available, e.g., from OligoEngine under the name pSuper RNAi System™.

Ribozyme molecules designed to catalytically cleave cellular mRNA transcripts can also be used to prevent translation of cellular mRNAs and expression of cellular polypeptides, or both (See, e.g., PCT International Publication WO90/11364, published Oct. 4, 1990; Sarver et al. (1990) Science 247:1222-1225 and U.S. Pat. No. 5,093,246). While ribozymes that cleave mRNA at site-specific recognition sequences can be used to destroy cellular mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well-known in the art and is described more fully in Haseloff and Gerlach (1988) Nature 334:585-591. The ribozyme may be engineered so that the cleavage recognition site is located near the 5′ end of cellular mRNAs; i.e., to increase efficiency and minimize the intracellular accumulation of non-functional mRNA transcripts.

The ribozymes of the methods presented herein also include RNA endoribonucleases (hereinafter “Cech-type ribozymes”) such as the one which occurs naturally in Tetrahymena thermophila (known as the IVS, or L-19 IVS RNA) and which has been extensively described by Thomas Cech and collaborators (Zaug et al. (1984) Science 224:574-578; Zaug et al. (1986) Science 231:470-475; Zaug et al. (1986) Nature 324:429-433; WO 88/04300; and Been et al. (1986) Cell 47:207-216). The Cech-type ribozymes have an eight base pair active site which hybridizes to a target RNA sequence whereafter cleavage of the target RNA takes place. The methods and compositions presented herein encompasses those Cech-type ribozymes which target eight base-pair active site sequences that are present in cellular genes.

As in the antisense approach, the ribozymes can be composed of modified oligonucleotides (e.g., for improved stability, targeting, etc.). A preferred method of delivery involves using a DNA construct “encoding” the ribozyme under the control of a strong constitutive pol III or pol II promoter, so that transfected cells will produce sufficient quantities of the ribozyme to destroy endogenous cellular messages and inhibit translation. Because ribozymes unlike antisense molecules, are catalytic, a lower intracellular concentration is required for efficiency.

Nucleic acid molecules to be used in triple helix formation for the inhibition of transcription of cellular genes are preferably single stranded and composed of deoxyribonucleotides. The base composition of these oligonucleotides should promote triple helix formation via Hoogsteen base pairing rules, which generally require sizable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in CGC triplets across the three strands in the triplex.

Alternatively, the potential sequences that can be targeted for triple helix formation may be increased by creating a so-called “switchback” nucleic acid molecule. Switchback molecules are synthesized in an alternating 5′-3′, 3′-5′ manner, such that they base pair with first one strand of a duplex and then the other, eliminating the necessity for a sizable stretch of either purines or pyrimidines to be present on one strand of a duplex.

Small nucleic acids (e.g., miRNAs, pre-miRNAs, pri-miRNAs, miRNA*, anti-miRNA, or a miRNA binding site, or a variant thereof), antisense oligonucleotides, ribozymes, and triple helix molecules of the methods and compositions presented herein may be prepared by any method known in the art for the synthesis of DNA and RNA molecules. These include techniques for chemically synthesizing oligodeoxyribonucleotides and oligoribonucleotides well-known in the art such as for example solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines.

Moreover, various well-known modifications to nucleic acid molecules may be introduced as a means of increasing intracellular stability and half-life. Possible modifications include but are not limited to the addition of flanking sequences of ribonucleotides or deoxyribonucleotides to the 5′ and/or 3′ ends of the molecule or the use of phosphorothioate or 2′ O-methyl rather than phosphodiesterase linkages within the oligodeoxyribonucleotide backbone. One of skill in the art will readily understand that polypeptides, small nucleic acids, and antisense oligonucleotides can be further linked to another peptide or polypeptide (e.g., a heterologous peptide), e.g., that serves as a means of protein detection. Non-limiting examples of label peptide or polypeptide moieties useful for detection in the invention include, without limitation, suitable enzymes such as horseradish peroxidase, alkaline phosphatase, beta-galactosidase, or acetylcholinesterase; epitope tags, such as FLAG, MYC, HA, or HIS tags; fluorophores such as green fluorescent protein; dyes; radioisotopes; digoxygenin; biotin; antibodies; polymers; as well as others known in the art, for example, in Principles of Fluorescence Spectroscopy, Joseph R. Lakowicz (Editor), Plenum Pub Corp, 2nd edition (July 1999).

The present invention also contemplates well-known methods for genetically modifying the genome of an organism or cell to modify the expression and/or activity of one or more DNA damage checkpoints and/or DNA damage repair genes without contacting the organism or cell with agent once the genetic modification has been completed. For example, cancer cells can be genetically modified using recombinant techniques in order to modulate the expression and/or activity of one or more DNA damage checkpoints and/or DNA damage repair genes, such that no agent needs to contact the cancer cells in order for the expression and/or activity one or more DNA damage checkpoints and/or DNA damage repair genes to be modulated. For example, targeted or untargeted gene knockout methods can be used, such as to recombinantly engineer subject cancer cell ex vivo prior to infusion into the subject. For example, the target DNA in the genome can be manipulated by deletion, insertion, and/or mutation using retroviral insertion, artificial chromosome techniques, gene insertion, random insertion with tissue specific promoters, gene targeting, transposable elements and/or any other method for introducing foreign DNA or producing modified DNA/modified nuclear DNA. Other modification techniques include deleting DNA sequences from a genome and/or altering nuclear DNA sequences. Nuclear DNA sequences, for example, may be altered by site-directed mutagenesis. Such methods generally use host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein. Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection, or electroporation. Suitable methods for transforming or transfecting host cells can be found in Sambrook, et al. (supra), and other laboratory manuals. For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Preferred selectable markers include those which confer resistance to drugs, such as G418, hygromycin and methotrexate. Cells stably transfected with the introduced nucleic acid can be identified by drug selection (e.g., cells that have incorporated the selectable marker gene will survive, while the other cells die).

Similarly, the CRISPR-Cas system can be used for precise editing of genomic nucleic acids (e.g., for creating null mutations). In such embodiments, the CRISPR guide RNA and/or the Cas enzyme may be expressed. For example, a vector containing only the guide RNA can be administered to an animal or cells transgenic for the Cas9 enzyme. Similar strategies may be used (e.g., designer zinc finger, transcription activator-like effectors (TALEs) or homing meganucleases). Such systems are well-known in the art (see, for example, U.S. Pat. No. 8,697,359; Sander and Joung (2014) Nat. Biotech. 32:347-355; Hale et al. (2009) Cell 139:945-956; Karginov and Hannon (2010) Mol. Cell 37:7; U.S. Pat. Publ. 2014/0087426 and 2012/0178169; Boch et al. (2011) Nat. Biotech. 29:135-136; Boch et al. (2009) Science 326:1509-1512; Moscou and Bogdanove (2009) Science 326:1501; Weber et al. (2011) PLoS One 6:e19722; Li et al. (2011) Nucl. Acids Res. 39:6315-6325; Zhang et al. (2011) Nat. Biotech. 29:149-153; Miller et al. (2011) Nat. Biotech. 29:143-148; Lin et al. (2014) Nucl. Acids Res. 42:e47). Such genetic strategies can use constitutive expression systems or inducible expression systems according to well-known methods in the art.

In some embodiments, the cancer cells are non-replicative. In certain embodiments, the cancer cells are non-replicative due to irradiation (e.g., γ and/or UV irradiation), and/or administration of an agent rendering cell replication incompetent (e.g., compounds that disrupt the cell membrane, inhibitors of DNA replication, inhibitors of spindle formation during cell division, etc.). Typically a minimum dose of about 3500 rads radiation is sufficient, although doses up to about 30,000 rads are acceptable. For example, the cancer cells may be irradiated to suppress cell proliferation before administration of the cancer vaccine to reduce the risk of giving rise to new neoplastic lesions. It is understood that irradiation is only one way to render the cells non-replicative, and that other methods which result in cancer cells incapable of cell division but that retain the ability to trigger the STING-dependent antitumor immunity upon PARP inhibition are included in the present invention.

c. PARP Inhibitor-Induced DNA Breaks

In one embodiment, DNA-repair deficient cancer cells described herein are contacted with a PARP inhibitor to induced DNA breaks. The PARP inhibitor is selected from the group consisting of olaparib, rucaparib, niraparib, veliparib (ABT-888), talazoparib (BMN 673), iniparib (BSI-201), E7449, INO-1001, AZD2461, ME0328, TNKS49, TNKS22, JW55, PJ34, INO-1001, WIKI4, NU 1025, DR 2313, BYK 49187, BYK 204165, MK-4827, UPF 1069, A-966492, 4-HQN, EB47, MK-4827 hydrochloride, MK-4827 tosylate, and MK-4827 racemate. PARP inhibitor-induced DNA breaks may comprise double-strand DNA breaks and/or single-strand DNA breaks. The DNA-repair deficient cancer cells described herein may not be able to repair the excess DNA breaks induced by PARP inhibitors, and thereby may release DNA fragments from the cells. The cancer cells may be contacted with a PARP inhibitor alone in vitro, in vivo, and/or ex vivo. In one embodiment, the cancer cells are contacted with a PARP inhibitor in vitro or ex vivo, and then the cancer cells are administered to a subject without administration of the PARP inhibitor to the subject in vivo. In another embodiment, the cancer cells are administered to a subject, wherein the PARP inhibitor is administered to the subject to thereby contact the cancer cells in vivo. In still another embodiment, the cancer cells are contacted with a PARP inhibitor in vitro or ex vivo, and then the cancer cells are administered to a subject with administration of the PARP inhibitor to the subject in vivo. The PARP inhibitor may be administered to the subject before, after, and/or concurrently with administration of the cancer cells. In some embodiments, the cancer cells are contacted with the PARP inhibitor in combination with an immune checkpoint blockade in vitro, in vivo, and/or ex vivo. The subject may be administered with an immune checkpoint blockade before, after, and/or concurrently with administration of the cancer vaccine.

### III. Subjects

In one embodiment, the subject for whom a cancer vaccine comprising DNA repair-deficient cancer cells, wherein the cancer cells are contacted with a PARP inhibitor to induce DNA breaks is administered, or whose predicted likelihood of efficacy of the cancer vaccine for treating a cancer is determined, is a mammal (e.g., rat, primate, non-human mammal, domestic animal, such as a dog, cat, cow, horse, and the like), and is preferably a human. In another embodiment, the subject is an animal model of cancer. For example, the animal model can be an orthotopic xenograft animal model of a human-derived cancer or allograft of syngeneic cancer models.

In another embodiment of the methods of the present invention, the subject has not undergone treatment, such as chemotherapy, radiation therapy, targeted therapy, and/or immunotherapies. In still another embodiment, the subject has undergone treatment, such as chemotherapy, radiation therapy, targeted therapy, and/or immunotherapies.

In certain embodiments, the subject has had surgery to remove cancerous or precancerous tissue. In other embodiments, the cancerous tissue has not been removed, e.g., the cancerous tissue may be located in an inoperable region of the body, such as in a tissue that is essential for life, or in a region where a surgical procedure would cause considerable risk of harm to the patient.

The methods of the present invention can be used to determine the responsiveness to the cancer vaccine for treating a cancer.

### IV. Methods of Treatment

The present invention provides for both prophylactic and therapeutic methods of treating a subject at risk of (or susceptible to) a cancer. The cancer may be a solid or hematological cancer. In certain embodiments, the cancer is ovarian cancer or breast cancer.

a. Prophylactic Methods

In one aspect, the present invention provides a method for preventing a subject afflicted with cancer, by administering to the subject a therapeutically effective amount of a cancer vaccine comprising DNA repair-deficient cancer cells, wherein the cancer cells are contacted with a PARP inhibitor to induce DNA breaks. Administration of a prophylactic agent can occur prior to the manifestation of symptoms characteristic of cancer, such that a cancer is prevented or, alternatively, delayed in its progression.

b. Therapeutic Methods Another aspect of the present invention pertains to methods treating a subject afflicted with cancer, by administering to the subject a therapeutically effective amount of a cancer vaccine comprising DNA repair-deficient cancer cells, wherein the cancer cells are contacted with a PARP inhibitor to induce DNA breaks.

As described below and in some embodiments, a cancer vaccine comprising DNA repair-deficient cancer cells, wherein the cancer cells are contacted with a PARP inhibitor to induce DNA breaks is administered to a subject. Thus, the cancer cells will have an immunocompatibility relationship to the subject host and any such relationship is contemplated for use according to the present invention. For example, the cancer cells can be syngeneic. The term “syngeneic” can refer to the state of deriving from, originating in, or being members of the same species that are genetically identical, particularly with respect to antigens or immunological reactions. These include identical twins having matching MHC types. Thus, a “syngeneic transplant” refers to transfer of cells from a donor to a recipient who is genetically identical to the donor or is sufficiently immunologically compatible as to allow for transplantation without an undesired adverse immunogenic response (e.g., such as one that would work against interpretation of immunological screen results described herein).

A syngeneic transplant can be “autologous” if the transferred cells are obtained from and transplanted to the same subject. An “autologous transplant” refers to the harvesting and reinfusion or transplant of a subject's own cells or organs. Exclusive or supplemental use of autologous cells may eliminate or reduce many adverse effects of administration of the cells back to the host, particular graft versus host reaction.

A syngeneic transplant can be “matched allogeneic” if the transferred cells are obtained from and transplanted to different members of the same species yet have sufficiently matched major histocompatibility complex (MHC) antigens to avoid an adverse immunogenic response. Determining the degree of MHC mismatch may be accomplished according to standard tests known and used in the art. For instance, there are at least six major categories of MHC genes in humans, identified as being important in transplant biology. HLA-A, HLA-B, HLA-C encode the HLA class I proteins while HLA-DR, HLA-DQ, and HLA-DP encode the HLA class II proteins. Genes within each of these groups are highly polymorphic, as reflected in the numerous HLA alleles or variants found in the human population, and differences in these groups between individuals is associated with the strength of the immune response against transplanted cells. Standard methods for determining the degree of MHC match examine alleles within HLA-B and HLA-DR, or HLA-A, HLA-B and HLA-DR groups. Thus, tests may be made of at least 4, and even 5 or 6 MHC antigens within the two or three HLA groups, respectively. In serological MHC tests, antibodies directed against each HLA antigen type are reacted with cells from one subject (e.g., donor) to determine the presence or absence of certain MHC antigens that react with the antibodies. This is compared to the reactivity profile of the other subject (e.g., recipient). Reaction of the antibody with an MHC antigen is typically determined by incubating the antibody with cells, and then adding complement to induce cell lysis (i.e., lymphocytotoxicity testing). The reaction is examined and graded according to the amount of cells lysed in the reaction (see, for example, Mickelson and Petersdorf (1999) Hematopoietic Cell Transplantation, Thomas, E. D. et al. eds., pg 28-37, Blackwell Scientific, Malden, Mass.). Other cell-based assays include flow cytometry using labeled antibodies or enzyme linked immunoassays (ELISA). Molecular methods for determining MHC type are well-known and generally employ synthetic probes and/or primers to detect specific gene sequences that encode the HLA protein. Synthetic oligonucleotides may be used as hybridization probes to detect restriction fragment length polymorphisms associated with particular HLA types (Vaughn (2002) Method. Mol. Biol. MHC Protocol. 210:45-60). Alternatively, primers may be used for amplifying the HLA sequences (e.g., by polymerase chain reaction or ligation chain reaction), the products of which may be further examined by direct DNA sequencing, restriction fragment polymorphism analysis (RFLP), or hybridization with a series of sequence specific oligonucleotide primers (SSOP) (Petersdorf et al. (1998) Blood 92:3515-3520; Morishima et al. (2002) Blood 99:4200-4206; and Middleton and Williams (2002) Method. Mol. Biol. MHC Protocol. 210:67-112).

A syngeneic transplant can be “congenic” if the transferred cells and cells of the subject differ in defined loci, such as a single locus, typically by inbreeding. The term “congenic” refers to deriving from, originating in, or being members of the same species, where the members are genetically identical except for a small genetic region, typically a single genetic locus (i.e., a single gene). A “congenic transplant” refers to transfer of cells or organs from a donor to a recipient, where the recipient is genetically identical to the donor except for a single genetic locus. For example, CD45 exists in several allelic forms and congenic mouse lines exist in which the mouse lines differ with respect to whether the CD45.1 or CD45.2 allelic versions are expressed.

By contrast, “mismatched allogeneic” refers to deriving from, originating in, or being members of the same species having non-identical major histocompatibility complex (MHC) antigens (i.e., proteins) as typically determined by standard assays used in the art, such as serological or molecular analysis of a defined number of MHC antigens, sufficient to elicit adverse immunogenic responses. A “partial mismatch” refers to partial match of the MHC antigens tested between members, typically between a donor and recipient. For instance, a “half mismatch” refers to 50% of the MHC antigens tested as showing different MHC antigen type between two members. A “full” or “complete” mismatch refers to all MHC antigens tested as being different between two members.

Similarly, in contrast, “xenogeneic” refers to deriving from, originating in, or being members of different species, e.g., human and rodent, human and swine, human and chimpanzee, etc. A “xenogeneic transplant” refers to transfer of cells or organs from a donor to a recipient where the recipient is a species different from that of the donor.

In addition, cancer cells can be obtained from a single source or a plurality of sources (e.g., a single subject or a plurality of subjects). A plurality refers to at least two (e.g., more than one). In still another embodiment, the non-human mammal is a mouse. The animals from which cell types of interest are obtained may be adult, newborn (e.g., less than 48 hours old), immature, or in utero. Cell types of interest may be primary cancer cells, cancer stem cells, established cancer cell lines, immortalized primary cancer cells, and the like. In certain embodiments, the immune systems of host subjects can be engineered or otherwise elected to be immunological compatible with transplanted cancer cells. For example, in one embodiment, the subject may be “humanized” in order to be compatible with human cancer cells. The term “immune-system humanized” refers to an animal, such as a mouse, comprising human HSC lineage cells and human acquired and innate immune cells, survive without being rejected from the host animal, thereby allowing human hematopoiesis and both acquired and innate immunity to be reconstituted in the host animal. Acquired immune cells include T cells and B cells. Innate immune cells include macrophages, granulocytes (basophils, eosinophils, neutrophils), DCs, NK cells and mast cells. Representative, non-limiting examples include SCID-hu, Hu-PBL-SCID, Hu-SRC-SCID, NSG (NOD-SCID IL2r-gamma(null) lack an innate immune system, B cells, T cells, and cytokine signaling), NOG (NOD-SCID IL2r-gamma(truncated)), BRG (BALB/c-Rag2(null)IL2r-gamma(null)), and H2dRG (Stock-H2d-Rag2(null)IL2r-gamma(null)) mice (see, for example, Shultz et al. (2007) Nat. Rev. Immunol. 7:118; Pearson et al. (2008) Curr. Protocol. Immunol. 15:21; Brehm et al. (2010) Clin. Immunol. 135:84-98; McCune et al. (1988) Science 241:1632-1639, U.S. Pat. No. 7,960,175, and U.S. Pat. Publ. 2006/0161996), as well as related null mutants of immune-related genes like Rag1 (lack B and T cells), Rag2 (lack B and T cells), TCR alpha (lack T cells), perforin (cD8+ T cells lack cytotoxic function), FoxP3 (lack functional CD4+ T regulatory cells), IL2rg, or Prfl, as well as mutants or knockouts of PD-1, PD-L1, Tim3, and/or 2B4, allow for efficient engraftment of human immune cells in and/or provide compartment-specific models of immunocompromised animals like mice (see, for example, PCT Publ. WO2013/062134). In addition, NSG-CD34+ (NOD-SCID IL2r-gamma(null) CD34+) humanized mice are useful for studying human gene and tumor activity in animal models like mice.

As used herein, “obtained” from a biological material source means any conventional method of harvesting or partitioning a source of biological material from a donor. For example, biological material may obtained from a solid tumor, a blood sample, such as a peripheral or cord blood sample, or harvested from another body fluid, such as bone marrow or amniotic fluid. Methods for obtaining such samples are well-known to the artisan. In the present invention, the samples may be fresh (i.e., obtained from a donor without freezing). Moreover, the samples may be further manipulated to remove extraneous or unwanted components prior to expansion. The samples may also be obtained from a preserved stock. For example, in the case of cell lines or fluids, such as peripheral or cord blood, the samples may be withdrawn from a cryogenically or otherwise preserved bank of such cell lines or fluid. Such samples may be obtained from any suitable donor.

The obtained populations of cells may be used directly or frozen for use at a later date. A variety of mediums and protocols for cryopreservation are known in the art. Generally, the freezing medium will comprise DMSO from about 5-10%, 10-90% serum albumin, and 50-90% culture medium. Other additives useful for preserving cells include, by way of example and not limitation, disaccharides such as trehalose (Scheinkonig et al. (2004) Bone Marrow Transplant. 34:531-536), or a plasma volume expander, such as hetastarch (i.e., hydroxyethyl starch). In some embodiments, isotonic buffer solutions, such as phosphate-buffered saline, may be used. An exemplary cryopreservative composition has cell-culture medium with 4% HSA, 7.5% dimethyl sulfoxide (DMSO), and 2% hetastarch. Other compositions and methods for cryopreservation are well-known and described in the art (see, e.g., Broxmeyer et al. (2003) Proc. Natl. Acad. Sci. U.S.A. 100:645-650). Cells are preserved at a final temperature of less than about −135° C.

c. Combination Therapy

The cancer vaccine can be administered in combination therapy with, e.g., chemotherapeutic agents, hormones, antiangiogens, radiolabelled, compounds, or with surgery, cryotherapy, and/or radiotherapy. The preceding treatment methods can be administered in conjunction with other forms of conventional therapy (e.g., standard-of-care treatments for cancer well-known to the skilled artisan), either consecutively with, pre- or post-conventional therapy. For example, the cancer vaccine can be administered with a therapeutically effective dose of chemotherapeutic agent. In another embodiment, the cancer vaccine is administered in conjunction with chemotherapy to enhance the activity and efficacy of the chemotherapeutic agent. The Physicians' Desk Reference (PDR) discloses dosages of chemotherapeutic agents that have been used in the treatment of various cancers. The dosing regimen and dosages of these aforementioned chemotherapeutic drugs that are therapeutically effective will depend on the particular cancer being treated, the extent of the disease and other factors familiar to the physician of skill in the art, and can be determined by the physician.

The cancer vaccine can also be administered in combination with targeted therapy, e.g., immunotherapy. The term “targeted therapy” refers to administration of agents that selectively interact with a chosen biomolecule to thereby treat cancer. For example, targeted therapy regarding the inhibition of immune checkpoint inhibitor is useful in combination with the methods of the present invention. The term “immune checkpoint inhibitor” means a group of molecules on the cell surface of CD4+ and/or CD8+ T cells that fine-tune immune responses by down-modulating or inhibiting an anti-tumor immune response. Immune checkpoint proteins are well-known in the art and include, without limitation, CTLA-4, PD-1, VISTA, B7-H2, B7-H3, PD-L1, B7-H4, B7-H6, 2B4, ICOS, HVEM, PD-L2, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, and A2aR (see, for example, WO 2012/177624). Inhibition of one or more immune checkpoint inhibitors can block or otherwise neutralize inhibitory signaling to thereby upregulate an immune response in order to more efficaciously treat cancer. In some embodiments, the cancer vaccine is administered in combination with one or more inhibitors of immune checkpoints, such as PD1, PD-L1, and/or CD47 inhibitors.

Immunotherapy is one form of targeted therapy that may comprise, for example, the use of additional cancer vaccines and/or sensitized antigen presenting cells. For example, an oncolytic virus is a virus that is able to infect and lyse cancer cells, while leaving normal cells unharmed, making them potentially useful in cancer therapy. Replication of oncolytic viruses both facilitates tumor cell destruction and also produces dose amplification at the tumor site. They may also act as vectors for anticancer genes, allowing them to be specifically delivered to the tumor site. The immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). For example, anti-VEGF and mTOR inhibitors are known to be effective in treating renal cell carcinoma. Immunotherapy can also focus on using the cytotoxic lymphocyte-recognized epitopes of cancer cell lines. Alternatively, antisense polynucleotides, ribozymes, RNA interference molecules, triple helix polynucleotides and the like, can be used to selectively modulate biomolecules that are linked to the initiation, progression, and/or pathology of a tumor or cancer.

The term “untargeted therapy” refers to administration of agents that do not selectively interact with a chosen biomolecule yet treat cancer. Representative examples of untargeted therapies include, without limitation, chemotherapy, gene therapy, and radiation therapy.

In one embodiment, chemotherapy is used. Chemotherapy includes the administration of a chemotherapeutic agent. Such a chemotherapeutic agent may be, but is not limited to, those selected from among the following groups of compounds: platinum compounds, cytotoxic antibiotics, antimetabolities, anti-mitotic agents, alkylating agents, arsenic compounds, DNA topoisomerase inhibitors, taxanes, nucleoside analogues, plant alkaloids, and toxins; and synthetic derivatives thereof. Exemplary compounds include, but are not limited to, alkylating agents: cisplatin, treosulfan, and trofosfamide; plant alkaloids: vinblastine, paclitaxel, docetaxol; DNA topoisomerase inhibitors: teniposide, crisnatol, and mitomycin; anti-folates: methotrexate, mycophenolic acid, and hydroxyurea; pyrimidine analogs: 5-fluorouracil, doxifluridine, and cytosine arabinoside; purine analogs: mercaptopurine and thioguanine; DNA antimetabolites: 2′-deoxy-5-fluorouridine, aphidicolin glycinate, and pyrazoloimidazole; and antimitotic agents: halichondrin, colchicine, and rhizoxin. Compositions comprising one or more chemotherapeutic agents (e.g., FLAG, CHOP) may also be used. FLAG comprises fludarabine, cytosine arabinoside (Ara-C) and G-CSF. CHOP comprises cyclophosphamide, vincristine, doxorubicin, and prednisone. The foregoing examples of chemotherapeutic agents are illustrative, and are not intended to be limiting.

In another embodiment, radiation therapy is used. The radiation used in radiation therapy can be ionizing radiation. Radiation therapy can also be gamma rays, X-rays, or proton beams. Examples of radiation therapy include, but are not limited to, external-beam radiation therapy, interstitial implantation of radioisotopes (1-125, palladium, iridium), radioisotopes such as strontium-89, thoracic radiation therapy, intraperitoneal P-32 radiation therapy, and/or total abdominal and pelvic radiation therapy. For a general overview of radiation therapy, see Hellman, Chapter 16: Principles of Cancer Management: Radiation Therapy, 6th edition, 2001, DeVita et al., eds., J. B. Lippencott Company, Philadelphia. The radiation therapy can be administered as external beam radiation or teletherapy wherein the radiation is directed from a remote source. The radiation treatment can also be administered as internal therapy or brachytherapy wherein a radioactive source is placed inside the body close to cancer cells or a tumor mass. Also encompassed is the use of photodynamic therapy comprising the administration of photosensitizers, such as hematoporphyrin and its derivatives, Vertoporfin (BPD-MA), phthalocyanine, photosensitizer Pc4, demethoxy-hypocrellin A; and 2BA-2-DMHA.

In another embodiment, hormone therapy is used. Hormonal therapeutic treatments can comprise, for example, hormonal agonists, hormonal antagonists (e.g., flutamide, bicalutamide, tamoxifen, raloxifene, leuprolide acetate (LUPRON), LH-RH antagonists), inhibitors of hormone biosynthesis and processing, and steroids (e.g., dexamethasone, retinoids, deltoids, betamethasone, cortisol, cortisone, prednisone, dehydrotestosterone, glucocorticoids, mineralocorticoids, estrogen, testosterone, progestins), vitamin A derivatives (e.g., all-trans retinoic acid (ATRA)); vitamin D3 analogs; antigestagens (e.g., mifepristone, onapristone), or antiandrogens (e.g., cyproterone acetate).

In another embodiment, hyperthermia, a procedure in which body tissue is exposed to high temperatures (up to 106° F.) is used. Heat may help shrink tumors by damaging cells or depriving them of substances they need to live. Hyperthermia therapy can be local, regional, and whole-body hyperthermia, using external and internal heating devices. Hyperthermia is almost always used with other forms of therapy (e.g., radiation therapy, chemotherapy, and biological therapy) to try to increase their effectiveness. Local hyperthermia refers to heat that is applied to a very small area, such as a tumor. The area may be heated externally with high-frequency waves aimed at a tumor from a device outside the body. To achieve internal heating, one of several types of sterile probes may be used, including thin, heated wires or hollow tubes filled with warm water; implanted microwave antennae; and radiofrequency electrodes. In regional hyperthermia, an organ or a limb is heated. Magnets and devices that produce high energy are placed over the region to be heated. In another approach, called perfusion, some of the patient's blood is removed, heated, and then pumped (perfused) into the region that is to be heated internally. Whole-body heating is used to treat metastatic cancer that has spread throughout the body. It can be accomplished using warm-water blankets, hot wax, inductive coils (like those in electric blankets), or thermal chambers (similar to large incubators). Hyperthermia does not cause any marked increase in radiation side effects or complications. Heat applied directly to the skin, however, can cause discomfort or even significant local pain in about half the patients treated. It can also cause blisters, which generally heal rapidly.

In still another embodiment, photodynamic therapy (also called PDT, photoradiation therapy, phototherapy, or photochemotherapy) is used for the treatment of some types of cancer. It is based on the discovery that certain chemicals known as photosensitizing agents can kill one-celled organisms when the organisms are exposed to a particular type of light. PDT destroys cancer cells through the use of a fixed-frequency laser light in combination with a photosensitizing agent. In PDT, the photosensitizing agent is injected into the bloodstream and absorbed by cells all over the body. The agent remains in cancer cells for a longer time than it does in normal cells. When the treated cancer cells are exposed to laser light, the photosensitizing agent absorbs the light and produces an active form of oxygen that destroys the treated cancer cells. Light exposure must be timed carefully so that it occurs when most of the photosensitizing agent has left healthy cells but is still present in the cancer cells. The laser light used in PDT can be directed through a fiber-optic (a very thin glass strand). The fiber-optic is placed close to the cancer to deliver the proper amount of light. The fiber-optic can be directed through a bronchoscope into the lungs for the treatment of lung cancer or through an endoscope into the esophagus for the treatment of esophageal cancer. An advantage of PDT is that it causes minimal damage to healthy tissue. However, because the laser light currently in use cannot pass through more than about 3 centimeters of tissue (a little more than one and an eighth inch), PDT is mainly used to treat tumors on or just under the skin or on the lining of internal organs. Photodynamic therapy makes the skin and eyes sensitive to light for 6 weeks or more after treatment. Patients are advised to avoid direct sunlight and bright indoor light for at least 6 weeks. If patients must go outdoors, they need to wear protective clothing, including sunglasses. Other temporary side effects of PDT are related to the treatment of specific areas and can include coughing, trouble swallowing, abdominal pain, and painful breathing or shortness of breath. In December 1995, the U.S. Food and Drug Administration (FDA) approved a photosensitizing agent called porfimer sodium, or Photofrin®, to relieve symptoms of esophageal cancer that is causing an obstruction and for esophageal cancer that cannot be satisfactorily treated with lasers alone. In January 1998, the FDA approved porfimer sodium for the treatment of early non-small cell lung cancer in patients for whom the usual treatments for lung cancer are not appropriate. The National Cancer Institute and other institutions are supporting clinical trials (research studies) to evaluate the use of photodynamic therapy for several types of cancer, including cancers of the bladder, brain, larynx, and oral cavity.

In yet another embodiment, laser therapy is used to harness high-intensity light to destroy cancer cells. This technique is often used to relieve symptoms of cancer such as bleeding or obstruction, especially when the cancer cannot be cured by other treatments. It may also be used to treat cancer by shrinking or destroying tumors. The term “laser” stands for light amplification by stimulated emission of radiation. Ordinary light, such as that from a light bulb, has many wavelengths and spreads in all directions. Laser light, on the other hand, has a specific wavelength and is focused in a narrow beam. This type of high-intensity light contains a lot of energy. Lasers are very powerful and may be used to cut through steel or to shape diamonds. Lasers also can be used for very precise surgical work, such as repairing a damaged retina in the eye or cutting through tissue (in place of a scalpel). Although there are several different kinds of lasers, only three kinds have gained wide use in medicine: Carbon dioxide (CO₂) laser—This type of laser can remove thin layers from the skin's surface without penetrating the deeper layers. This technique is particularly useful in treating tumors that have not spread deep into the skin and certain precancerous conditions. As an alternative to traditional scalpel surgery, the CO₂ laser is also able to cut the skin. The laser is used in this way to remove skin cancers. Neodymium:yttrium-aluminum-garnet (Nd:YAG) laser—Light from this laser can penetrate deeper into tissue than light from the other types of lasers, and it can cause blood to clot quickly. It can be carried through optical fibers to less accessible parts of the body. This type of laser is sometimes used to treat throat cancers. Argon laser—This laser can pass through only superficial layers of tissue and is therefore useful in dermatology and in eye surgery. It also is used with light-sensitive dyes to treat tumors in a procedure known as photodynamic therapy (PDT). Lasers have several advantages over standard surgical tools, including: Lasers are more precise than scalpels. Tissue near an incision is protected, since there is little contact with surrounding skin or other tissue. The heat produced by lasers sterilizes the surgery site, thus reducing the risk of infection. Less operating time may be needed because the precision of the laser allows for a smaller incision. Healing time is often shortened; since laser heat seals blood vessels, there is less bleeding, swelling, or scarring. Laser surgery may be less complicated. For example, with fiber optics, laser light can be directed to parts of the body without making a large incision. More procedures may be done on an outpatient basis. Lasers can be used in two ways to treat cancer: by shrinking or destroying a tumor with heat, or by activating a chemical—known as a photosensitizing agent—that destroys cancer cells. In PDT, a photosensitizing agent is retained in cancer cells and can be stimulated by light to cause a reaction that kills cancer cells. CO₂ and Nd:YAG lasers are used to shrink or destroy tumors. They may be used with endoscopes, tubes that allow physicians to see into certain areas of the body, such as the bladder. The light from some lasers can be transmitted through a flexible endoscope fitted with fiber optics. This allows physicians to see and work in parts of the body that could not otherwise be reached except by surgery and therefore allows very precise aiming of the laser beam. Lasers also may be used with low-power microscopes, giving the doctor a clear view of the site being treated. Used with other instruments, laser systems can produce a cutting area as small as 200 microns in diameter—less than the width of a very fine thread. Lasers are used to treat many types of cancer. Laser surgery is a standard treatment for certain stages of glottis (vocal cord), cervical, skin, lung, vaginal, vulvar, and penile cancers. In addition to its use to destroy the cancer, laser surgery is also used to help relieve symptoms caused by cancer (palliative care). For example, lasers may be used to shrink or destroy a tumor that is blocking a patient's trachea (windpipe), making it easier to breathe. It is also sometimes used for palliation in colorectal and anal cancer. Laser-induced interstitial thermotherapy (LITT) is one of the most recent developments in laser therapy. LITT uses the same idea as a cancer treatment called hyperthermia; that heat may help shrink tumors by damaging cells or depriving them of substances they need to live. In this treatment, lasers are directed to interstitial areas (areas between organs) in the body. The laser light then raises the temperature of the tumor, which damages or destroys cancer cells.

The immunotherapy and/or cancer therapy may be administered before, after, or concurrently with the cancer vaccine described herein. The duration and/or dose of treatment with the cancer vaccine may vary according to the particular cancer vaccine, or the particular combinatory therapy. An appropriate treatment time for a particular cancer therapeutic agent will be appreciated by the skilled artisan. The invention contemplates the continued assessment of optimal treatment schedules for each cancer therapeutic agent, where the phenotype of the cancer of the subject as determined by the methods of the invention is a factor in determining optimal treatment doses and schedules.

### V. Clinical Efficacy

Clinical efficacy can be measured by any method known in the art. For example, the response to an cancer therapy (e.g., a cancer vaccine comprising DNA repair-deficient cancer cells, wherein the cancer cells are contacted with a PARP inhibitor to induce DNA breaks), relates to any response of the cancer, e.g., a tumor, to the therapy, preferably to a change in tumor mass and/or volume after initiation of neoadjuvant or adjuvant chemotherapy. Tumor response may be assessed in a neoadjuvant or adjuvant situation where the size of a tumor after systemic intervention can be compared to the initial size and dimensions as measured by CT, PET, mammogram, ultrasound or palpation and the cellularity of a tumor can be estimated histologically and compared to the cellularity of a tumor biopsy taken before initiation of treatment. Response may also be assessed by caliper measurement or pathological examination of the tumor after biopsy or surgical resection. Response may be recorded in a quantitative fashion like percentage change in tumor volume or cellularity or using a semi-quantitative scoring system such as residual cancer burden (Symmans et al. (2007) J. Clin. Oncol. 25:4414-4422) or Miller-Payne score (Ogston et al. (2003) Breast (Edinburgh, Scotland) 12:320-327) in a qualitative fashion like “pathological complete response” (pCR), “clinical complete remission” (cCR), “clinical partial remission” (cPR), “clinical stable disease” (cSD), “clinical progressive disease” (cPD) or other qualitative criteria. Assessment of tumor response may be performed early after the onset of neoadjuvant or adjuvant therapy, e.g., after a few hours, days, weeks or preferably after a few months. A typical endpoint for response assessment is upon termination of neoadjuvant chemotherapy or upon surgical removal of residual tumor cells and/or the tumor bed.

In some embodiments, clinical efficacy of the therapeutic treatments described herein may be determined by measuring the clinical benefit rate (CBR). The clinical benefit rate is measured by determining the sum of the percentage of patients who are in complete remission (CR), the number of patients who are in partial remission (PR) and the number of patients having stable disease (SD) at a time point at least 6 months out from the end of therapy. The shorthand for this formula is CBR=CR+PR+SD over 6 months. In some embodiments, the CBR for a particular cancer vaccine therapeutic regimen is at least 25%, 30%, 35%, 40%, 45%, 50%, 55%, 60%, 65%, 70%, 75%, 80%, 85%, or more.

Additional criteria for evaluating the response to cancer therapy (e.g., a cancer vaccine comprising DNA repair-deficient cancer cells, wherein the cancer cells are contacted with a PARP inhibitor to induce DNA breaks) are related to “survival,” which includes all of the following: survival until mortality, also known as overall survival (wherein said mortality may be either irrespective of cause or tumor related); “recurrence-free survival” (wherein the term recurrence shall include both localized and distant recurrence); metastasis free survival; disease free survival (wherein the term disease shall include cancer and diseases associated therewith). The length of said survival may be calculated by reference to a defined start point (e.g., time of diagnosis or start of treatment) and end point (e.g., death, recurrence or metastasis). In addition, criteria for efficacy of treatment can be expanded to include response to chemotherapy, probability of survival, probability of metastasis within a given time period, and probability of tumor recurrence.

For example, in order to determine appropriate threshold values, a particular agent encompassed by the present invention can be administered to a population of subjects and the outcome can be correlated to biomarker measurements that were determined prior to administration of any cancer therapy (e.g., a cancer vaccine comprising DNA repair-deficient cancer cells, wherein the cancer cells are contacted with a PARP inhibitor to induce DNA breaks). The outcome measurement may be pathologic response to therapy given in the neoadjuvant setting. Alternatively, outcome measures, such as overall survival and disease-free survival can be monitored over a period of time for subjects following cancer therapy (e.g., a cancer vaccine comprising DNA repair-deficient cancer cells, wherein the cancer cells are contacted with a PARP inhibitor to induce DNA breaks) for whom biomarker measurement values are known. In certain embodiments, the same doses of the agent are administered to each subject. In related embodiments, the doses administered are standard doses known in the art for the agent. The period of time for which subjects are monitored can vary. For example, subjects may be monitored for at least 2, 4, 6, 8, 10, 12, 14, 16, 18, 20, 25, 30, 35, 40, 45, 50, 55, or 60 months. Biomarker measurement threshold values that correlate to outcome of a cancer therapy (e.g., a cancer vaccine comprising DNA repair-deficient cancer cells, wherein the cancer cells are contacted with a PARP inhibitor to induce DNA breaks) can be determined using methods such as those described in the Examples section.

### VI. Pharmaceutical Compositions and Administration

For cancer vaccine of present invention, cancer cells can be administered at 0.1×10⁶, 0.2×10⁶, 0.3×10⁶, 0.4×10⁶, 0.5×10⁶, 0.6×10⁶, 0.7×10⁶, 0.8×10⁶, 0.9×10⁶, 1.0×10⁶, 5.0×10⁶, 1.0×10⁷, 5.0×10⁷, 1.0×10⁸, 5.0×10⁸, or more, or any range in between or any value in between, cells per kilogram of subject body weight. The number of cells transplanted may be adjusted based on the desired level of engraftment in a given amount of time. Generally, 1×10⁵ to about 1×10⁹ cells/kg of body weight, from about 1×10⁶ to about 1×10⁸ cells/kg of body weight, or about 1×10⁷ cells/kg of body weight, or more cells, as necessary, may be transplanted. In some embodiment, transplantation of at least about 0.1×10⁶, 0.5×10⁶, 1.0×10⁶, 2.0×10⁶, 3.0×10⁶, 4.0×10⁶, or 5.0×10⁶ total cells relative to an average size mouse is effective.

Cancer vaccine can be administered in any suitable route as described herein, such as by infusion. Cancer vaccine can also be administered before, concurrently with, or after, other anti-cancer agents.

Administration can be accomplished using methods generally known in the art. Agents, including cells, may be introduced to the desired site by direct injection, or by any other means used in the art including, but are not limited to, intravascular, intracerebral, parenteral, intraperitoneal, intravenous, epidural, intraspinal, intrasternal, intra-articular, intra-synovial, intrathecal, intra-arterial, intracardiac, or intramuscular administration. For example, subjects of interest may be engrafted with the transplanted cells by various routes. Such routes include, but are not limited to, intravenous administration, subcutaneous administration, administration to a specific tissue (e.g., focal transplantation), injection into the femur bone marrow cavity, injection into the spleen, administration under the renal capsule of fetal liver, and the like. In certain embodiment, the cancer vaccine of the present invention is injected to the subject intratumorally or subcutaneously. Cells may be administered in one infusion, or through successive infusions over a defined time period sufficient to generate a desired effect. Exemplary methods for transplantation, engraftment assessment, and marker phenotyping analysis of transplanted cells are well-known in the art (see, for example, Pearson et al. (2008) Curr. Protoc. Immunol. 81:15.21.1-15.21.21; Ito et al. (2002) Blood 100:3175-3182; Traggiai et al. (2004) Science 304:104-107; Ishikawa et al. Blood (2005) 106:1565-1573; Shultz et al. (2005) J. Immunol. 174:6477-6489; and Holyoake et al. (1999) Exp. Hematol. 27:1418-1427).

Two or more cell types can be combined and administered, such as cancer vaccine and adoptive cell transfer of stem cells, cancer vaccine and other cell-based vaccines, and the like. For example adoptive cell-based immunotherapies can be combined with the cancer vaccine of the present invention. Well-known adoptive cell-based immunotherapeutic modalities, including, without limitation, irradiated autologous or allogeneic tumor cells, tumor lysates or apoptotic tumor cells, antigen-presenting cell-based immunotherapy, dendritic cell-based immunotherapy, adoptive T cell transfer, adoptive CAR T cell therapy, autologous immune enhancement therapy (AIET), cancer vaccines, and/or antigen presenting cells. Such cell-based immunotherapies can be further modified to express one or more gene products to further modulate immune responses, such as expressing cytokines like GM-CSF, and/or to express tumor-associated antigen (TAA) antigens, such as Mage-1, gp-100, and the like. The ratio of cancer cells in the cancer vaccine described herein to other cell types can be 1:1, but can modulated in any amount desired (e.g., 1:1, 1.1:1, 1.2:1, 1.3:1, 1.4:1, 1.5:1, 2:1, 2.5:1, 3:1, 3.5:1, 4:1, 4.5:1, 5:1, 5.5:1, 6:1, 6.5:1, 7:1, 7.5:1, 8:1, 8.5:1, 9:1, 9.5:1, 10:1, or greater).

Engraftment of transplanted cells may be assessed by any of various methods, such as, but not limited to, tumor volume, cytokine levels, time of administration, flow cytometric analysis of cells of interest obtained from the subject at one or more time points following transplantation, and the like. For example, a time-based analysis of waiting 1, 2, 3, 4, 5, 6, 7, 8, 9, 10, 11, 12, 13, 14, 15, 16, 17, 18, 19, 20, 21, 22, 23, 24, 25, 26, 27, 28 days or can signal the time for tumor harvesting. Any such metrics are variables that can be adjusted according to well-known parameters in order to determine the effect of the variable on a response to anti-cancer immunotherapy. In addition, the transplanted cells can be co-transplanted with other agents, such as cytokines, extracellular matrices, cell culture supports, and the like.

In addition, anti-cancer agents (e.g., PARP inhibitors and/or immune checkpoint inhibitors) of the present invention can be administered to subjects or otherwise applied outside of a subject body in a biologically compatible form suitable for pharmaceutical administration. By “biologically compatible form suitable for administration in vivo” is meant a form to be administered in which any toxic effects are outweighed by the therapeutic effects. Administration of an anti-cancer agent as described herein can be in any pharmacological form including a therapeutically active amount of an agent alone or in combination with a pharmaceutically acceptable carrier. The phrase “therapeutically-effective amount” as used herein means that amount of an agent that is effective for producing some desired therapeutic effect, e.g., cancer treatment, at a reasonable benefit/risk ratio.

Administration of a therapeutically active amount of the therapeutic composition of the present invention is defined as an amount effective, at dosages and for periods of time necessary, to achieve the desired result. For example, a therapeutically active amount of an agent may vary according to factors such as the disease state, age, sex, and weight of the individual, and the ability of peptide to elicit a desired response in the individual. Dosage regimens can be adjusted to provide the optimum therapeutic response. For example, several divided doses can be administered daily or the dose can be proportionally reduced as indicated by the exigencies of the therapeutic situation.

A combination dosage form or simultaneous administration of single agents can result in effective amounts of each desired modulatory agent present in the patient at the same time.

The therapeutic agents described herein can be administered in a convenient manner such as by injection (subcutaneous, intravenous, etc.), oral administration, inhalation, transdermal application, or rectal administration. Depending on the route of administration, the active compound can be coated in a material to protect the compound from the action of enzymes, acids and other natural conditions which may inactivate the compound. For example, for administration of agents, by other than parenteral administration, it may be desirable to coat the agent with, or co-administer the agent with, a material to prevent its inactivation.

An agent can be administered to an individual in an appropriate carrier, diluent or adjuvant, co-administered with enzyme inhibitors or in an appropriate carrier such as liposomes. Pharmaceutically acceptable diluents include saline and aqueous buffer solutions. Adjuvant is used in its broadest sense and includes any immune stimulating compound such as interferon. Adjuvants contemplated herein include resorcinols, non-ionic surfactants such as polyoxyethylene oleyl ether and n-hexadecyl polyethylene ether. Enzyme inhibitors include pancreatic trypsin inhibitor, diisopropylfluorophosphate (DEEP) and trasylol. Liposomes include water-in-oil-in-water emulsions as well as conventional liposomes (Sterna et al. (1984) J. Neuroimmunol. 7:27).

The agent may also be administered parenterally or intraperitoneally. Dispersions can also be prepared in glycerol, liquid polyethylene glycols, and mixtures thereof, and in oils. Under ordinary conditions of storage and use, these preparations may contain a preservative to prevent the growth of microorganisms.

Pharmaceutical compositions of agents suitable for injectable use include sterile aqueous solutions (where water soluble) or dispersions and sterile powders for the extemporaneous preparation of sterile injectable solutions or dispersion. In all cases the composition will preferably be sterile and must be fluid to the extent that easy syringeability exists. It will preferably be stable under the conditions of manufacture and storage and preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it is preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating an agent of the invention in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the agent plus any additional desired ingredient from a previously sterile-filtered solution thereof.

When the agent is suitably protected, as described above, the protein can be orally administered, for example, with an inert diluent or an assimilable edible carrier. As used herein “pharmaceutically acceptable carrier” includes any and all solvents, dispersion media, coatings, antibacterial and antifungal agents, isotonic and absorption delaying agents, and the like. The use of such media and agents for pharmaceutically active substances is well-known in the art. Except insofar as any conventional media or agent is incompatible with the active compound, use thereof in the therapeutic compositions is contemplated. Supplementary active compounds can also be incorporated into the compositions.

It is especially advantageous to formulate parenteral compositions in dosage unit form for ease of administration and uniformity of dosage. “Dosage unit form”, as used herein, refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by, and directly dependent on, (a) the unique characteristics of the active compound and the particular therapeutic effect to be achieved, and (b) the limitations inherent in the art of compounding such an active compound for the treatment of sensitivity in individuals.

### VII. Kits

The present invention also encompasses kits. For example, the kit can comprise DNA repair-deficient cancer cells modified as described herein, PARP inhibitors, immune checkpoint inhibitors, and combinations thereof, packaged in a suitable container and can further comprise instructions for using such reagents. The kit may also contain other components, such as administration tools packaged in a separate container.

Other embodiments of the present invention are described in the following Examples. The present invention is further illustrated by the following examples which should not be construed as further limiting. The contents of all references, patents and published patent applications cited throughout this application, as well as the Figures, are incorporated herein by reference.

### EXAMPLES

### Example 1: Materials and Methods for Examples 2-11

a. Experimental Model and Subject Details

i. Mice

All animal experiments described herein were performed according to the animal protocols approved by the DFCI Institutional Animal Care and Use Committee. Brca1loxP/loxP mouse line was kindly provided by Dr. Jos Jonkers's laboratory (Netherlands Cancer Institute, Amsterdam, The Netherlands). Trp53loxP/loxP mouse line was obtained from National Cancer Institute Mouse Repository. PtenloxP/loxP mouse line was kindly provided by Dr. Hong Wu (Peking University, Beijing, China). All these mouse lines were backcrossed for more than 10 generations to the FVB/N background before intercrossed to make homozygous mouse lines. STING knock out mice (C57BL/6J-Tmem173gt/J, Stock No: 017537) were purchased from the Jackson Laboratory.

ii. Cell Lines

The 293T cell line was obtained from the American Type Culture Collection (ATCC, Manassas, Va.) and cultured in DMEM supplemented with 10% FBS and 100 μg/ml penicillin-streptomycin. PPM and PBM cells were generated from primary ovarian tumors and cultured in MOT media (DMEM/F12, 0.6% FBS, 10 ng/ml EGF, hydrocortisone 1 μg/ml, cholera toxin 1 ng/ml, 100 μg/ml penicillin-streptomycin, 5 μM Y27632). UWB1.289 and UWB1.289+BRCA1 were purchased from ATCC and cultured in complete growth medium (50% ATCC-formulated RPMI-1640 medium, 50% MEGM medium and 3% fetal bovine serum). All cell lines were cultured at 37° C. in a fully humidified atmosphere of 5% CO2.

b. Development of Brca1-Deficient and -Proficient HGSOC Mouse Model (GEMMs)

All animal experiments described in this study were performed according to the animal protocols approved by the DFCI Institutional Animal Care and Use Committee. Brca1^{loxP/loxP} [FVB/N] mice, bearing loxP sites in introns 4 and 13 of the Brca1 gene, and Trp53^{loxP/loxP}[FVB/N] mice bearing loxP in introns 2 and 10 of the p53 gene, were obtained from Dr. Jos Jonkers's laboratory (Netherlands Cancer Institute, Amsterdam, The Netherlands). These mice were intercrossed through multiple generations to create a colony of homozygous Trp53^{loxP/loxP}/Brca1^{loxP/loxP} double conditional knockout mice. Ovaries were harvested from 8-week-old female Trp53^{loxP/loxP}/Brca1^{loxP/loxP} mice. The ovaries were washed twice with phosphate-buffered and incubated in DMEM/F12 (Ham's) medium containing collagenase and dispase (stem cell technologies) for 40 min at 37° C. The epithelial cells were pelleted by centrifugation at 800×g and cultured in DMEM/F12 (Life Technologies) supplemented with 4% (vol/vol) FBS, 1% penicillin/streptomycin, 10 ng/mL EGF, 5 μg/mL insulin, 5 Cpg/mL transferrin, and 5 ng/mL sodium selenite. Ovarian surface epithelial (OSE) cells were cultured for 48 hr and then introduced with Adeno-Cre (University of Iowa) and lentiviruses expressing C-Myc gene (Addgene #36980) or control GFP. After two days of infection in vitro, OSE cells are aggregated and implanted into recipient mice. About 5×10⁵ OSE cells (Trp53^{-/-}; Brca1^{-/-}; c-Myc) were injected into the right ovarian bursal cavities of 6- to 7-week-old female nude mice. In each mouse, the left ovarian bursa was injected with GFP-expressing OSE cells (Trp53^{-/-}; Brca1^{-/-}; GFP). PBM (Trp53^{-/-}; Brca1^{-/-}; c-Myc) ovarian tumors developed within three to six months after implantation of the genetically engineered OSE cells. The primary ovarian tumors were digested and orthotopically transplanted to FVB/NJ mice. The PPM GEM model (Trp53^{-/-}; Pten^{-/-}; c-Myc) was developed with OSE cells isolated from homozygous Trp53^{loxP/loxP}/Pten^{loxP/loxP}(FVB/NJ) double conditional knockout mice using the same strategy for generation of the PBM GEM model described above. For histological analysis, tumor pieces were fixed in 10% Formalin overnight and transferred to 70% ethanol. Embedding, sectioning and H&E staining was performed by scientists at the Harvard rodent histopathology core facility. The histological characteristics of high grade ovarian tumor were confirmed by two independent pathologists at Harvard Medical School.

c. Lentiviral Production and Transduction

The 293T cell line was obtained from the American Type Culture Collection (ATCC, Manassas, Va.) and cultured in DMEM supplemented with 10% FBS and 100 jkg/ml penicillin-streptomycin. The pLenti-blasticidin-Luciferase vector or pWPXL-c-Myc were co-transfected with pCMV-delta8.9 and pVSVG at the ratio of 2:2:1 into HEK293T cells by PEI (1 μl/l) (4:1 to DNA). The medium was changed 24 hr after transfection and the viral supernatants were collected 48 hr later by filtering through a 0.45-μm filter and ultracentrifugation (SW28, 16,600 rpm, 2 h). Viral pellets were resuspended in RPMI-1640 media and aliquoted and stored at −80° C. for future use.

d. Tumor Growth and Treatment

Single cells were obtained by digestion of primary ovarian tumors in collagenase buffer and then cultured in MOT media (DMEM/F12, 0.6% FBS, 10 ng/ml EGF, hydrocortisone 1 μg/ml, cholera toxin 1 ng/ml, 100 μg/ml penicillin-streptomycin, 5 μM Y27632). Tumor cells were transduced with lentiviral vector encoding luciferase (pLenti-blasticidin-luciferase) and then subjected to 3 days antibiotic selection with blasticidin 2 μg/ml. These luciferased tumors were transplanted orthotopically into syngeneic FVB/NJ mice to generated tumors for drug evaluation. Olaparib (AZD2281) was used by diluting 100 mg/ml stocks in DMSO with 10% 2-hydroxyl-propyl-β-cyclodextrine/PBS and administered daily by i.p. injection at dose of 50 mg/kg body weight. Mouse anti-PD-1 monoclonal antibody kindly provided by Gordon Freeman at DFCI was diluted in PBS (250 μg/100 μl/mouse) and injected by i.p. every 3 days. BX795 (Cat # HY-10514/CS-0259, MedChem Express) was prepared in DMSO (100 mg/ml) as stock solution and diluted in (25% PEG400+0.5% Tween)/PBS and administered daily by i.p. injection at dose of 30 mg/kg body weight. Anti-IFNAR1 antibody (Cat # BE0241; clone, MAR1-5A3; InVivoMab) was diluted in PBS (200 μg/100 μl/mouse) and injected by i.p. every 3 days. Tumor-bearing mice were equivalently divided into control and treatment groups according to the luminescent intensity. The endpoints were determined by tumor burden and ascites. For the CD8 depletion experiment, mice were injected intraperitoneally with anti-CD8 antibody (400 μg; clone YTS 169.4, BioXcell) 24 and 48 h before beginning olaparib treatment (50 mg/kg/day) and every 4 days thereafter.

e. Bioluminescence Imaging

Mice were injected i.p. with D-luciferin (Gold Biotechnology) (˜120 mg/kg) and the luciferase signal was detected 10 min later using an IVIS® imaging system. Images were obtained and analyzed using Living Image® Software.

f. Flow Cytometry Analysis

Tumors were first mechanically disrupted by chopping, then chemically digested in collagenase buffer (8 mL DMEM, 0.1 mL 1M HEPES, 0.5 mL FBS, 0.2 mL Pen/Strep, 1 m, 10× collagenase/hyaluronidase (Stemcell Technologies), 0.2 mL of 1 mg/mL DNase I (Stemcell Technologies) at 37° C. for 45 min. Single-cell suspensions of spleen and lymph nodes were obtained by mashed through a 70 um strainer using the plunger of a 3 mL or 5 mL syringe. Single cells suspensions were treated with red blood lysis buffer (4 mL NH4Cl+1 mL PBS with 2% calf serum) and washed with FACS buffer. Single cells suspensions were incubated with LIVE/DEAD Fixable Aqua Dead Cell Stain (Life Technologies, Cat # L34965) for 30 min and then blocked with anti-CD16/32 (BioLegend, clone 93) for 20 min on ice. Samples were then incubated with appropriate antibodies for 30 min. on ice. The Foxp3 staining buffer set (eBioscience, Cat #00-5523-00) was applied for intracellular markers staining. For the intracellular cytokine analysis, cells were stimulated with Leukocyte Activation Cocktail (BD Biosciences, Cat #550583) at 37° C. for 5 hours prior to FACS staining. The following antibodies were used in the experiments and were purchased from BioLegend unless otherwise indicated: CD45 (clone 30-F11), TCRβ (clone H57-597), CD4 (clone RM4-5), CD8 (clone 53-6.7), CD44 (clone IM7), CD62L (MEL-14), CD25 (PC61), IFNγ (clone XMG1.2), TNFα(clone MP6-XT22), PD-1 (clone 29F.1A12), Tim-3 (clone RMT3-23), LAG-3 (clone C9B7W), CD11b (clone M1/70), CD11c (clone BM8), F4/80 (clone BM8), Gr-1 (clone RB6-BC5), Ly-6C (clone HK1.4), Ly-6G (clone 1A8), MHCII (clone M5/114.15.2), CD80 (clone 16-10A1), CD86 (clone GL-1), MHC1 (clone KH114). FoxP3 (clone FJK-16 s; eBioscience), Phospho-IRF-3 (Ser396) (clone D6O1M, Cell signaling technology), Phospho-TBK1/NAK (Ser172) (clone D52C2, Cell signaling technology), and LIVE/DEAD® Fixable Aqua Dead Cell Stain Kit (Life Technologies Cat L34965). Flow cytometry was performed on an LSR II (BD Biosciences) or LSRFortessa™ HTS (BD Biosciences) at the DFCI flow cytometry core facility, and all the data were analyzed using FlowJo software.

g. Cytosolic dsDNA Staining

PBM and PPM cells were cultured on chambered cell culture slides (BD Falcon). Cells were treated with 2.5 μM olaparib or vehicle control (DMSO) for 24 hours. Following the treatment, cells were incubated with culture media containing PicoGreen double-stranded DNA stain (200-fold dilution, Life Technologies). After one-hour incubation, cells were fixed with 4% paraformaldehyde in PBS (Santa Cruz Biotechnology) for 10 minutes. Cells were then washed twice with PBS and stained with 300 nM DAPI (Thermo Fisher Scientific) for 10 minutes. Coverslips were mounted using ProLong Diamond Antifade Mountant (Thermo Fisher Scientific). Staining was imaged using a Leica SP5X laser scanning confocal microscope.

h. Cytokine Array Analysis and ELISAs

Blood was obtained by retro-orbital sampling at intermediate time points or experimental end points. Blood cells and plasma/sera were separated by centrifugation at 1,500×g for 5 min. at 4° C. Plasmaisera were sent out to Eve Technologies for cytokine array analysis (Chemokine Array 31-Plex and Mouse Procarta IFN 2-plex Featured Assay). Plasma/sera were applied to ELISA according to manufacturer's instructions. For in vitro experiments, cell culture supernatants were obtained by centrifugation at 1,500×g for 5 min. at 4° C. to remove all debris and cells, and were then subjected to ELISA analysis. IFN-β was detected using the LEGEND MAX™ Mouse IFN-β ELISA Kit (BioLegend).

i. Generation of Brca-1 Deficient ID8 Cells and Tumors

Brca-1 deficient ID8 cells were generated using a CRISPR double nickase plasmid. ID8 cells cultured in a 6-well plate were transfected with 2 μg/well of BRCA1 double nickase plasmid (sc419362-NIC, Santa Cruz Biotechnology) or control double nickase plasmid (SC-437281, Santa Cruz Biotechnology) using lipofectamine 3000 (Invitrogen). 48 hrs post-transfection, cells were passaged onto a 10 cm plate. After 24 h, puromycin (3 μg/mL) was added to the culture for selection. Puromycin resistant cells were selected and expanded. Due to a lack of a reliable mouse BRCA1 antibody for Western blot, cells were analyzed by DNA sequencing to confirm the disruption of functional Brca1 allele. To generate ID8 tumors, cultured ID8 cells were harvested and resuspended in serum free DMEM. Cells were then mixed with Matrigel (Corning). A total volume of 0.1 ml containing 5×106 ID8 cells and 40% Matrigel were injected subcutaneously into the flank of C57BL/6J mice.

j. Measurement of IC50 Value in Tumor Cells

Tumor cells were seeded in 96-well plates at a density of 1000-3000/well and allowed to adhere overnight. Cells were then exposed to appropriate concentrations of therapeutic agents (or vehicle control) with continuous exposure for 72 h. Growth inhibition was measured by CellTiter Aqueous MTS reagent from Promega by comparing the absorbance at 490 nm of drug-treated cells to that of untreated controls set at 100%. 1C50 values were calculated using non-linear regression model (logarithmic inhibitor vs. normalized response-variable slope) in Graphpad Prism 7.

k. In Vitro Co-Culture of Tumor Cells and BMDCs

Bone marrow cells were isolated from FVB/NJ mice and cultured in RPMI-1640 containing 10% FBS and 20 ng/ml GM-CSF. BMDCs (bone marrow derived dendritic cells) were harvested for in vitro co-culture assay on day 7 to day 10. About 3×10⁵ PBM or PPM tumor cells were cultured in a 6-well plate for 24 hr. and then treated with DMSO or olaparib. After 24 hr incubation with DMSO, olaparib, or olaparib in combination with other drugs (cell cycle inhibitor or apoptosis inhibitor), drugs were removed and tumor cells were washed twice with PBS. BMDCs were added and co-cultured with PBM or PPM cells at a ratio of 1:1 in 1.5 ml culture media in the presence of GM-CSF (20 ng/ml) and lipofectamine (2 μl) for 24 hr. Co-cultured cells were harvested for flow cytometry analysis and floating cells (percentage of BMDCs is about 90%), or RNA analysis. Cell culture supernatants were collected for detection of IFN-β with the ELISA kit. BMDCs generated from C57BL/6J mice were applied for the coculture of BMDCs with ID8 tumor cells. Human dendritic cells purchased from Lonza were used for the coculture of human DCs with DMSO or olaparib-treated human ovarian cancer cells (ULTWB1.289 and UWB1.289+BRCA1).

1. RNA Extraction and Quantitative Real-Time PCR

About 50 to 100 mg of tumor samples were homogenized in 1 ml of TRIzol™ Reagent supplied with 200 μl of stainless steel beads and 0.2 ml of chloroform was added to the samples. Samples were vortexed vigorously for 15 seconds and then incubated at room temperature for 2 to 3 minutes. After centrifugation at 12,000×g for 15 minutes at 4° C., upper aqueous phase of the samples was carefully removed to a new tube and the total RNA was purified using an RNeasy® Mini Kit (Qiagen) according to manufactory's instruction. For the in vitro cultured cell samples, total RNA was isolated using an RNeasy® Mini Kit. The qPCR was performed on an Applied Biosystems 7300 machine after the total RNA was reverse transcribed to cDNA using the SuperScript® III First-Strand Synthesis System (Invitrogen). Primers used for qPCR were as follows:

p53-F 5'-CCCGAGTATCTGGAAGACAG-3',
p53-R 5'-ATAGGTCGGCGGTTCAT-3';
Brcal-F 5'-TGAAGACTGCTCGCAGAGTGATA-3';
Brcal-R 5'-AGCTTCCAGGTGAGCCATTTC-3';
Myc-F 5'-CAGAGGAGGAACGAGCTGAAGCGC-3',
Myc-R 5'-TTATGCACCAGAGTTTCGAAGCTGTTCG-3';
Pten-F 5'-AGACCATAACCCACCACAGC 3';
Pten-R 5'-TAGGGCCTCTTGTGCCTTTA-3';
IFNβ-F 5'-TCCGAGCAGAGATCTTCAGGAA-3',
IFNβ-R 5'-TGCAACCACCACTCATTCTGAG-3';
Cxcl10-F 5'-GCCGTCATTTTCTGCCTCA-3';
Cxcl10-R 5'-CGTCCTTGCGAGAGGGATC-3';
18SrRNA-F 5'-CTTAGAGGGACAAGTGGCG-3',
18SrRNA-R 5'-ACGCTGAGCCAGTCAGTGTA-3';
Gapdh-F 5'-ACAACTTTGGCATTGTGGAA-3',
Gapdh-R 5'-GATGCAGGGATGATGTTCTG-3'

m. Gene Set Enrichment Analyses (GSEA)

RNA was isolated from tumor samples as described above, sequenced on the ion Torrent platform per manufacturer's instructions using a custom AmpliSeq panel targeting 4604 murine genes relevant to our studies, and raw data was processed using the Torrent Suite and AmpliSeqRNA plugin to give read counts per gene. Differential gene expression analyses were carried out using DESeq2 with default parameters to obtain log 2 fold change (MAP) and adjusted p-values (Benjamini-Hochberg procedure) (Love et al. (2014) Genorme Biol. 15:550). Genes were ranked by log 2 fold change (MAP), and GSEA were carried out using the GSEAPreranked tool (Mootha et al. (2003) Nat. Genet. 34:267-273; Subramanian et al. (2005) Proc. Natl. Acad. Sci. USA 102:15545-15550). PCA were carried out using DESeq2.

n. Quantification and Statistical Analysis

Statistical analysis was performed using Prism 7 software (Graphpad Software Inc.). For all quantitative measurements, normal distribution was assumed, with t-tests performed, unpaired and two-sided. Two-tailed Student's t-test for normally distributed data and Mann-Whitney nonparametric test for skewed data that deviate from normality were used to compare two conditions. One-way ANOVA with Bonferroni's post-hoc test for normally distributed data and Kruskal-Wallis nonparametric test for skewed data were used to compare three or more means. Differences with P<0.05 were considered statistically significant. All data shown are representative two or more independent experiments, unless otherwise indicated.

o. Data Availability

Transcriptomic data that support the findings of this study have been deposited in the Gene Expression Omnibus under primary accession code GSE120500.

p. Key Resources Table

| REAGENT or RESOURCE | SOURCE | IDENTIFIER |
|---|---|---|
| Antibodies | | |
| FITC anti-mouse CD45 (clone 30-F11) | BioLegend | Cat# 103108 |
| FITC anti-human CD45 (clone HI30) | BioLegend | Cat# 304006 |
| PerCP/Cy5.5 anti-mouse TCR β chain (clone | BioLegend | Cat# 109228 |
| H57-597) | | |
| APC/Cy7 anti-mouse CD4 (clone RM4-5) | BioLegend | Cat# 100526 |
| Alexa Fluor 700 anti-mouse CD8a (clone 53-6.7) | BioLegend | Cat# 100730 |
| Brilliant Violet 605 anti-mouse/human CD44 | BioLegend | Cat# 103047 |
| (clone IM7) | | |
| Brilliant Violet 711 anti-mouse CD62L (clone | BioLegend | Cat# 104445 |
| MEL-14) | | |
| PE anti-mouse IFN-γ (clone XMG1.2) | BioLegend | Cat# 505808 |
| APC anti-mouse TNT-α (clone MP6-XT22) | BioLegend | Cat# 506308 |
| PE/Cy7 anti-mouse CD11c (clone N418) | BioLegend | Cat# 117318 |
| Brilliant Violet 650 anti-human CD11c (clone 3.9) | BioLegend | Cat# 301638 |
| APC/Cy7 anti-mouse I-A/I-E (clone M5/114.15.2) | BioLegend | Cat# 107628 |
| PE anti-mouse CD80 (clone 16-10A1) | BioLegend | Cat# 104708 |
| Pacific Blue anti-mouse CD86 (clone GL-1) | BioLegend | Cat# 105022 |
| Brilliant Violet 605 anti-mouse CD103 (clone | BioLegend | Cat# 121433 |
| 2E7) | | |
| Brilliant Violet 650 ™ anti-mouse/human CD11b | BioLegend | Cat# 101228 |
| (clone M1/70) | | |
| APC anti-mouse Ly-6C (clone HK1.4) | BioLegend | Cat# 128015 |
| Pacific Blue anti-mouse Ly-6G (clone 1A8) | BioLegend | Cat# 127612 |
| APC/Cy7 anti-human HLA-DR (clone L243) | BioLegend | Cat# 307618 |
| PE anti-mouse/human phospho-TBK1 (Ser172) | Cell Signaling Tech. | Cat# 13498S |
| (clone D52C2) | | |
| PE anti-mouse/human phospho-TBK1 (Ser172) | BD Biosciences | Cat# 558604 |
| (clone J133-587) | | |
| Alexa Fluor 647 anti-mouse/human phospho-IRF-3 | Cell Signaling Tech. | Cat# 10327S |
| (Ser396) (clone D6O1M) | | |
| PD-L1 antibody (10F.9G2) | BioLegend | Cat# 124321 |
| InVivoMAb anti-mouse CD8α neutralizing | BioXcell | Cat# BE0117 |
| antibody (clone YTS 169.4) | | |
| InVivoMAb anti-mouse IFNAR-2 neutralizing | BioXcell | Cat# BE0241 |
| antibody (clone MAR1-5A3) | | |
| Anti-PD-1 antibody (clone, 332.8H3) | Dr. Gordon | |
| | Freeman's lab at | |
| | Dana-Farber Cancer | |
| | Institute | |
| Bacterial and Virus Strains | | |
| Ad5CMVCre | University of Iowa | VVC-U of Iowa-5 |
| AdSCMVCre-eGFP | University of Iowa | VVC-U of Iowa-1174 |
| Chemicals, Peptides, and Recombinant Proteins | | |
| Olaparib | MedChem Express | Cat# HY-10162 |
| BX-795 hydrochloride | Sigma Aldrich | Cat# SML0694 |
| (2-Hydroxypropyl)-β-cyclodextrin (HPCD) | MedChem Express | Cat# HY-101103 |
| D-Luciferin, Potassium Salt | Gold BioTechnology | Cat# LUCK |
| Paraformaldehyde solution 4% in PBS | Santa Cruz | Cat# sc-281692 |
| 7-AAD Viability Staining Solution | BioLegend | Cat# 420404 |
| FITC Annexin V | BioLegend | Cat# 640906 |
| CDK1 inhibitor IV, RO-3306 | Calbiochem | Cat# 217699 |
| Mouse Recombinant GM-CSF | Stemcell | Cat# 78017.1 |
| | Technologies | |
| Collagenase/hyaluronidase | Stemcell | Cat# 07912 |
| | Technologies | |
| DNase I | Stemcell | Cat# 07900 |
| | Technologies | |
| Critical Commercial Assay | | |
| Quant-iT PicoGreen dsDNA Reagent | Life Technologies | Cat# P7581 |
| Mouse IFN beta ELISA Kit | Thermo Fisher | Cat# EN424001 |
| | Scientific | |
| Annexin V Binding Buffer | BioLegend | Cat# 422201 |
| MEGM Bullet Kit | Lonza | Cat# CC-3150 |
| Deposited Data | | |
| Transcriptome data | GEO | GSE120500 |
| Experimental Models: Cell Lines | | |
| UWB1.289 | American Type | Cat# CRL-2945 |
| | Culture Collection | |
| UWB1.289 + BRCA1 | American Tpe | Cat# CRL-2946 |
| | Culture Collection | |
| NHDC-Human Dendritic Cells | Lonza | Cat# CC-2701 |
| Experimental Models: Organisms/Strains | | |
| C57BL/6J-Tmem173gt/J | The Jackson | Stock No: 017537 |
| | Laboratory | |
| FVB/NJ mice | The Jackson | Stock No: 001800 |
| | Laboratory | |
| C57B6 mice | The Jackson | Stock No: 000664 |
| | Laboratory | |
| PBM | This paper | FVB/NJ |
| PPM | This paper | FVB/NJ |
| Recombinant DNA | | |
| BRCA1 Double Nickase Plasmid (m) | Santa Cruz | Cat# SC-419362- |
| | Biotechnology | NIC |
| Software and Algorithms | | |
| PRISM 7 software | Graphpad | https://www.graphpad.com/ |
| | | scientific-software/prism/ |
| GSEA (v2.2.2) | Broad Institute | http://software.broadinstitute.org/ |
| | | gsea/index.jsp |
| Flowjo (version 10.1)) | FlowJo, LLC | https://www.flowjo.com/ |
| | | solutions/flowjo/ |

### Example 2: Therapeutic Efficacy of PARP Inhibition for HR-Deficient Ovarian Cancer was Augmented by the Addition of Immune Checkpoint Blockade

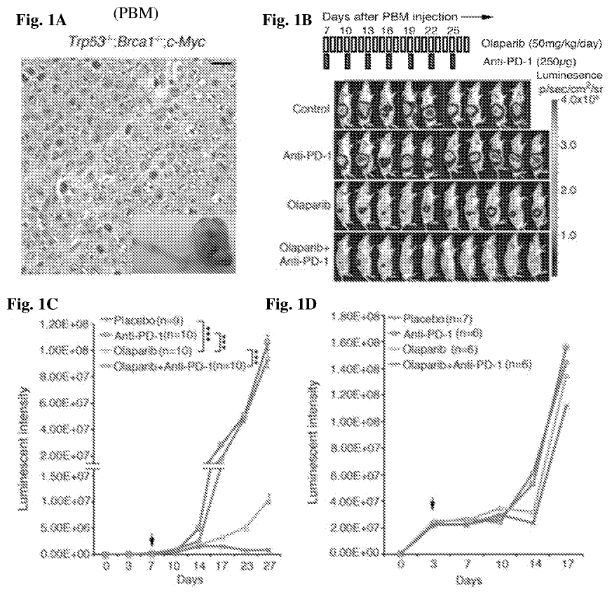

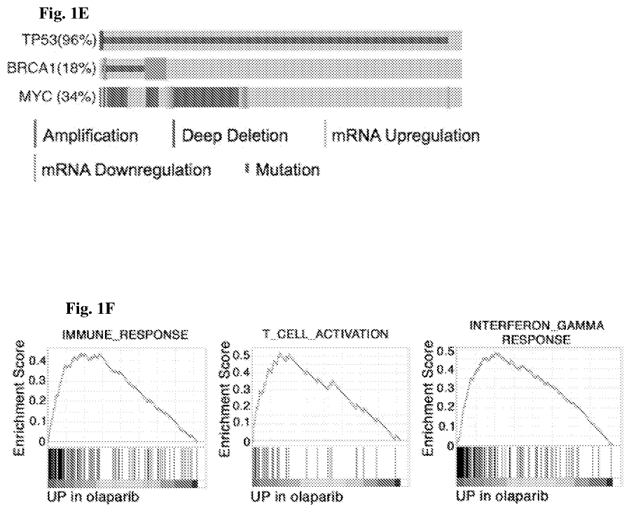

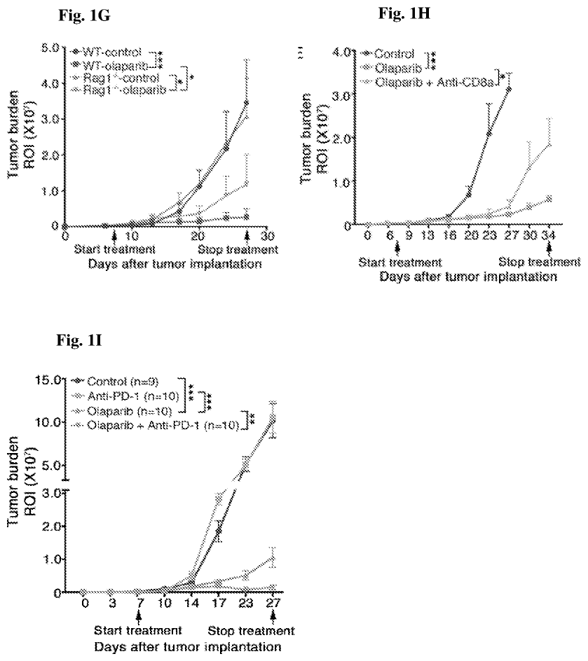

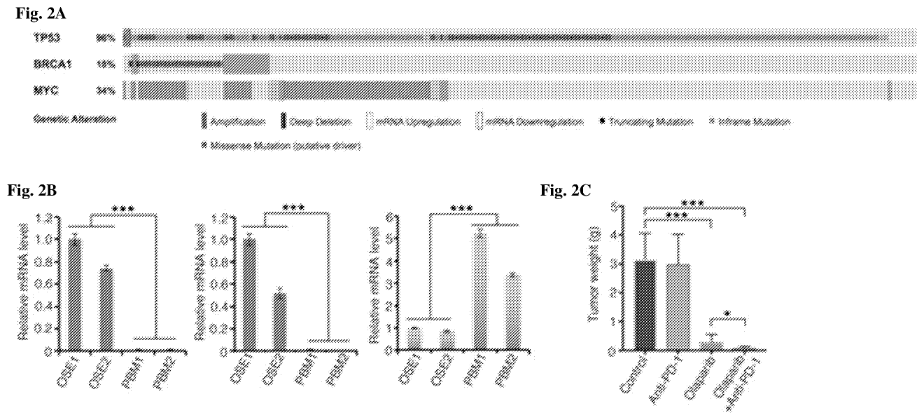

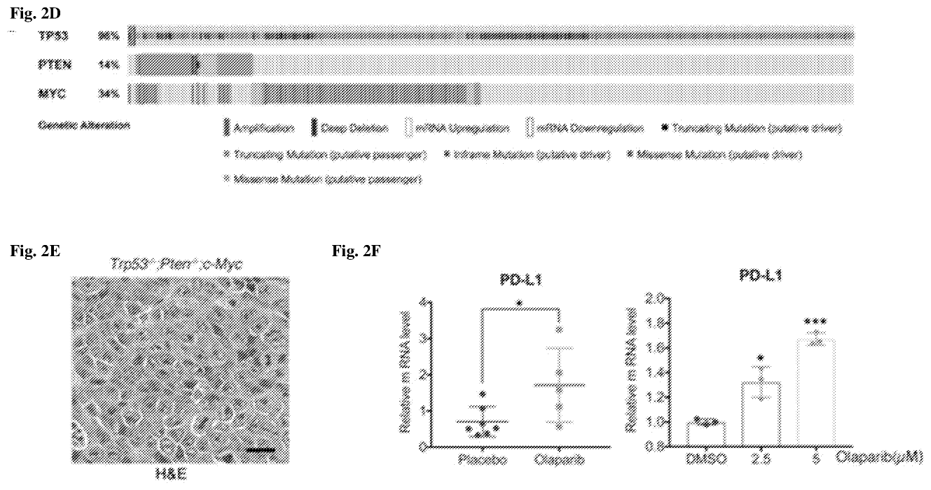

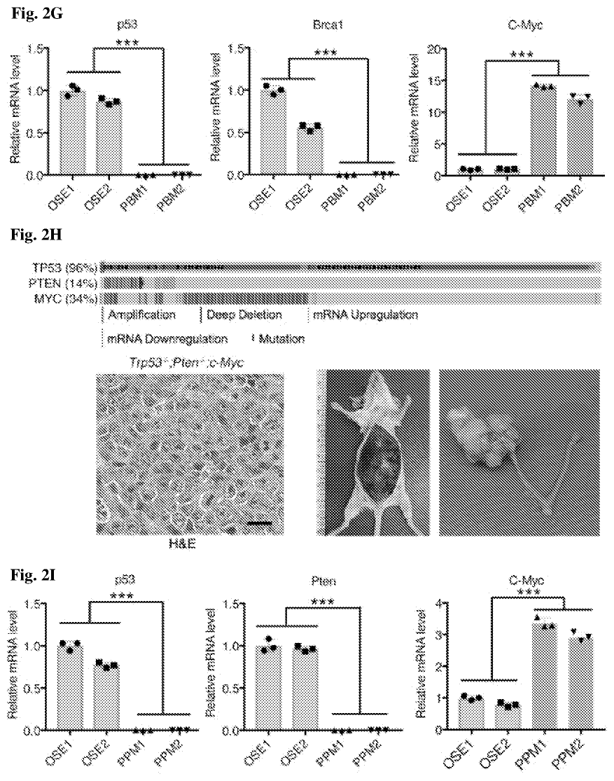

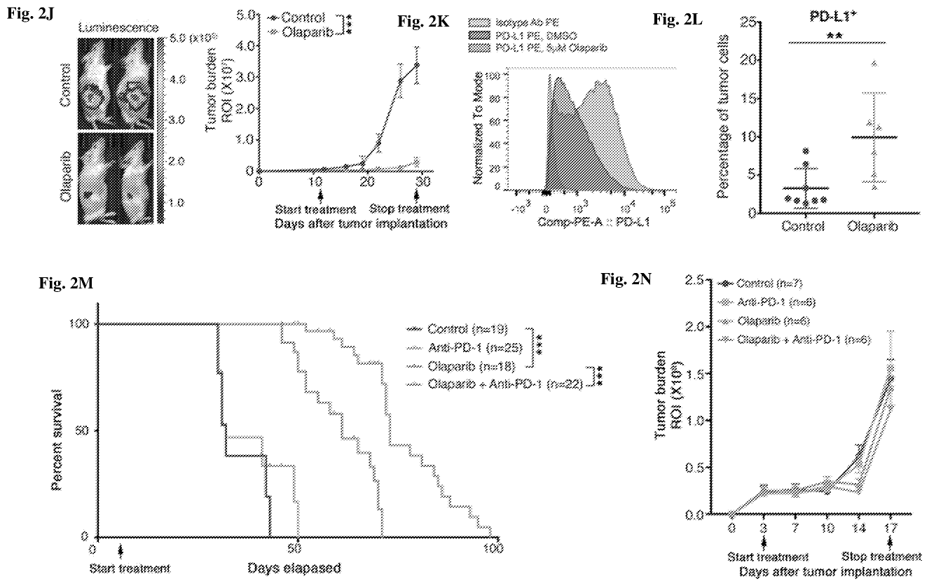

Therapeutic efficacy of olaparib in Brca1-deficient ovarian tumor involves T cell-mediated cytotoxicity, which is further enhanced by the addition of PD-1 blockade. The current understanding of molecular mechanism(s) underlying PARP inhibition for BRCA-deficient tumor is primarily described as cell autonomous synthetic lethality (O'Neil et al. (2017) Nat. Rev. Genet. 18:613-623). To explore the involvement of immune responses regarding PARP inhibition in HR-deficient cancer, a pair of syngeneic genetically engineered mouse models (GEMMs) of high-grade serous ovarian cancer (HGSOC) in the FVB background driven via either concurrent loss of p53 and Brca1 and overexpression of c-Myc (termed PBM, an HR-deficient model) or concurrent loss of p53 and Pten and overexpression of c-Myc (termed PPM, an HR-proficient model) was generated to reflect oncogenic events frequently found in human ovarian cancer (FIGS. 1A, 1E, 2A, 2B, 2D, and 2G-2I; The Cancer Genome Atlas Research Network (2011) Nature 474:609-615). Both PBM and PPM tumors display histological features resembling HGSOC in human tumors, characterized by glandular complexity, nuclear atypia with marked pleomorphism and stromal invasion (FIG. 1A; FIG. 2E; FIG. 2H; Vang et al. (2009) Adv. Anatom. Pathol. 16:267-282). PBM tumor cells expressing luciferase were engrafted into the ovarian bursa of a cohort of FVB female mice, which allows for the monitoring of tumor growth and immunological events upon olaparib treatment in a physiological tissue environment (FIG. 1B). Based on the vulnerability of ovarian cancers to agents that interrupt DNA repair, PARP inhibition has been shown to be effective for the treatment of ovarian cancer (Rafii et al. (2017) Oncotarget 8:47154-47160). In the PBM model, treatment of tumor bearing mice with olaparib significantly delayed tumor progression compared to mice with no treatment control group (FIG. 2J). Gene Set Enrichment Analysis (GSEA) was performed for a panel of 4604 cancer and immune-related genes in PBM tumor tissues harvested from tumor bearing mice after 18 days of treatment with olaparib or vehicle. Strikingly, GSEA showed markedly upregulated expression of genes associated with immune response, T-cell activation and interferon-γ (IFNγ) response in tumors treated with olaparib as compared to vehicle (FIG. 1F). To determine whether these immune responses play a role in the anti-tumor activity of olaparib in PBM in vivo, Rag1^{-/-} (FVB background) and wild-type FVB mice were engrafted with PBM tumors and treated tumor-bearing mice with olaparib. The results showed that the therapeutic effect of olaparib is partially abrogated (FIG. 1G), indicating that the adaptive immune system is indeed participated in the antitumor activity of olaparib. Next, anti-CD8a antibody was used in the allograft model of PBM in wild-type FVB host mice to show that olaparib-induced tumor inhibition was significantly mitigated by CD8 neutralization (FIG. 1H), demonstrating that cytotoxic T cell mediated cellular killing is important for the antitumor efficacy of olaparib.

While delayed tumor growth is obvious upon this PARP inhibition using olaparib, PBM tumors treated with olaparib highly enhanced expression of immune-inhibitory ligand PD-L1 on tumor cells (FIGS. 2F, 2K and 2L), which is consistent with clinical observation (Jiao et al. (2017) Clin. Cancer Res. 23:3711). Therefore, blockade of the PD1-PDL1 interaction within TME combined with olaparib treatment is believed to enhance anti-tumor immune response by precluding the therapy-induced inhibitory interaction between tumor and immune cells. Accordingly, another cohort of PBM-bearing mice was therefore subjected to the combination of olaparib and PD-1 blockade. While treatment of mice with anti-PD-1 antibody (Ab) had no effect on the growth of the PBM tumors, the combination of olaparib and anti-PD-1 Ab treatment resulted in sustained regression of tumor (FIGS. 1B-1C, 1I, and 2C), indicating that selective targeting of the immune inhibitory pathway(s) activated post to chemotherapy can lead to tumor eradication. Consistently, treatment with olaparib significantly prolonged the survival of PBM tumor-bearing mice and the survival was further extended by the addition of PD-1 blockade (FIG. 2M). These data indicate that, while olaparib is effective in treating PBM tumors, activation of immune inhibitory pathway(s) limits the effectiveness of PARP inhibition, which can be overcome by incorporating immune checkpoint blockade in the treatment regimen. Interestingly, however, all PBM-bearing mice treated with olaparib alone or in combination with PD-1 blockade eventually succumbed to the disease (FIG. 1M). In contrast, olaparib alone or combined with anti-PD-1 Ab treatment had little effect on the progression of Brca-proficient PPM tumors, indicating that molecular mechanisms associated with Brca deficiency in tumors determines the efficacy of this therapy (FIG. 1D and FIG. 2N). These results recapitulate the efficacy of PARP inhibition in the clinic for BRCA-deficient (HR-deficient) ovarian cancer (Ledermann (2016) Ann. Oncol. 27:i40-i44; Lord and Ashworth (2017) Science 355:1152-1158), and indicate that addition of immune checkpoint blockade augments the therapeutic efficacy of PARP inhibition. The results presented herein also indicate that, while multiple PARP inhibitors have been approved for the treatment of BRCA1-deficient ovarian cancers, eradication of this type of cancer remains a challenge.

### Example 3: Olaparib Treatment Triggered a Robust Antitumor Immunity in the PBM Tumor Microenvironment Involving Both Lymphoid and Myeloid Derived Cells

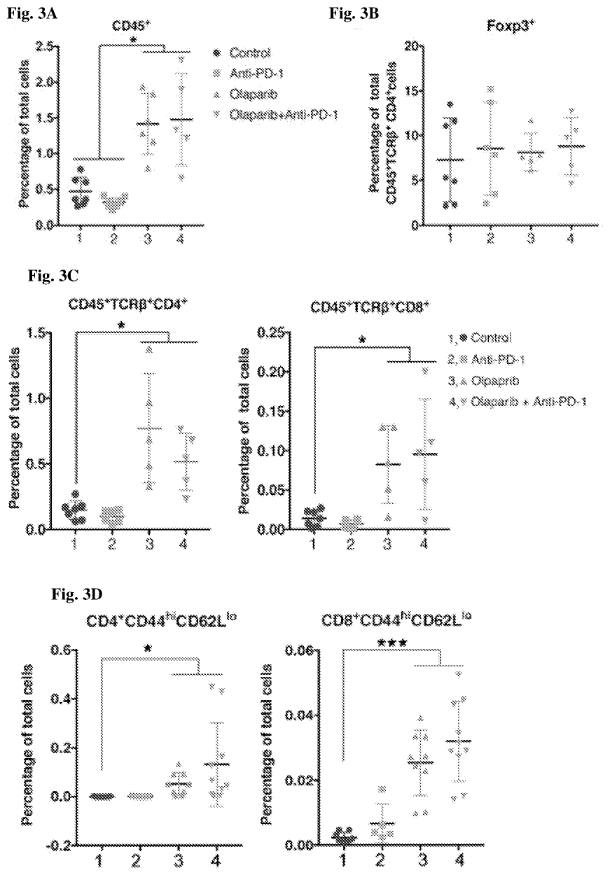

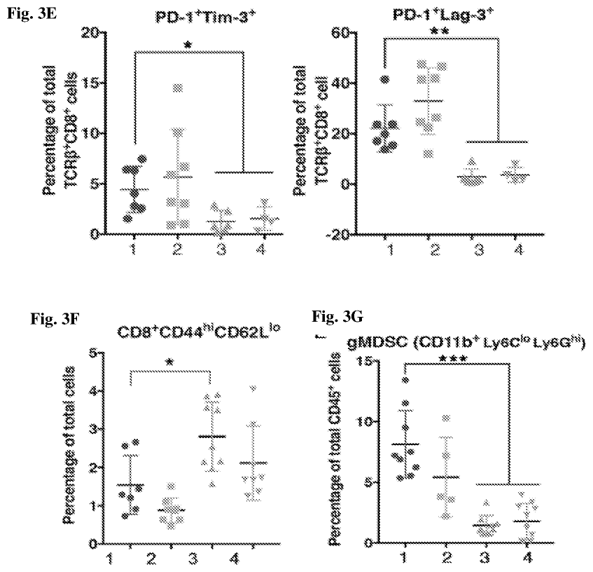

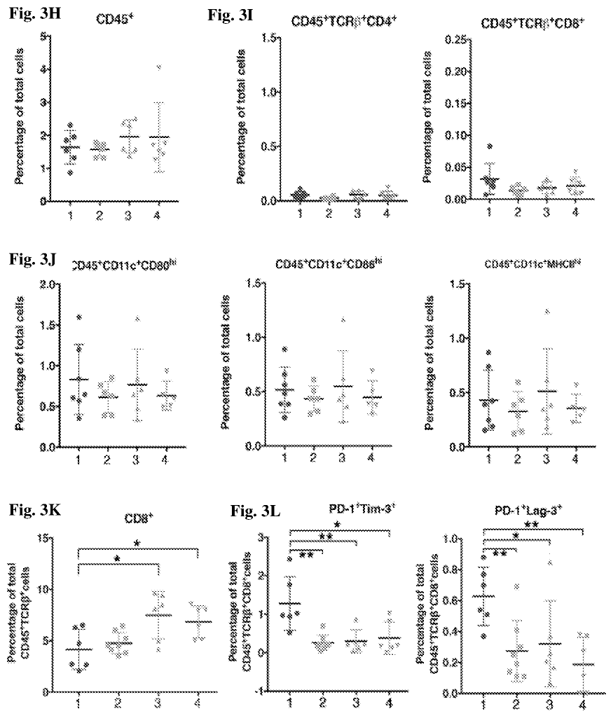

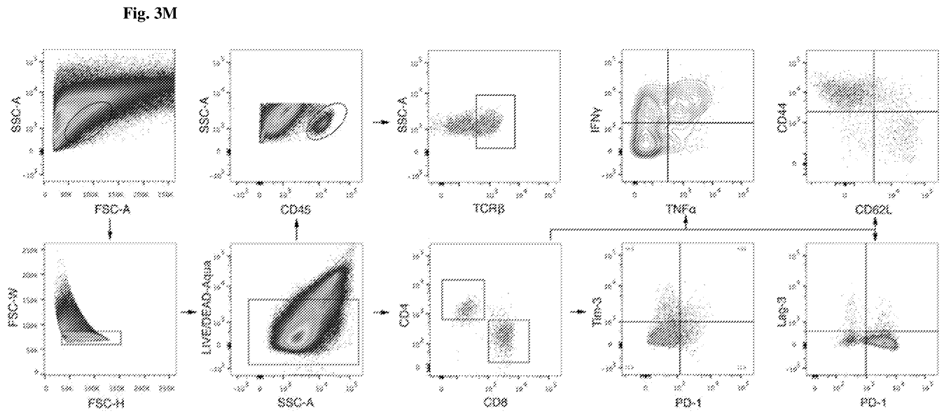

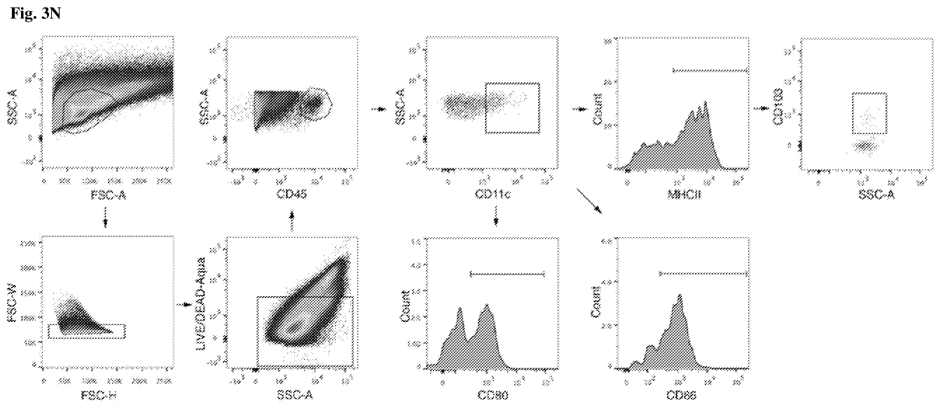

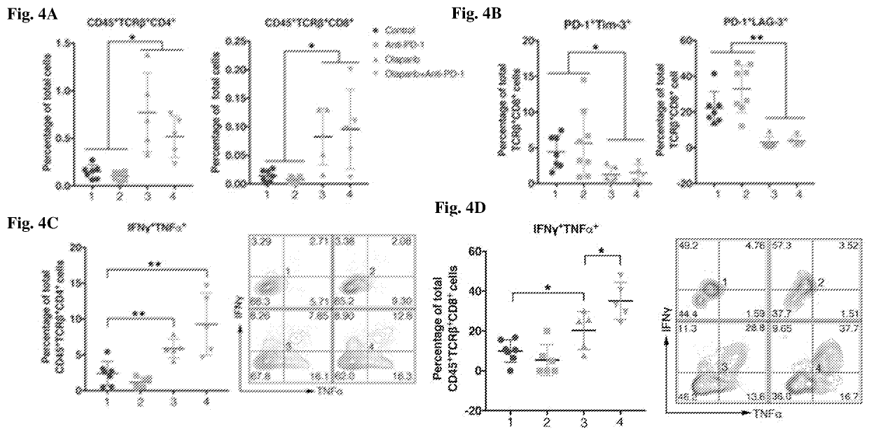

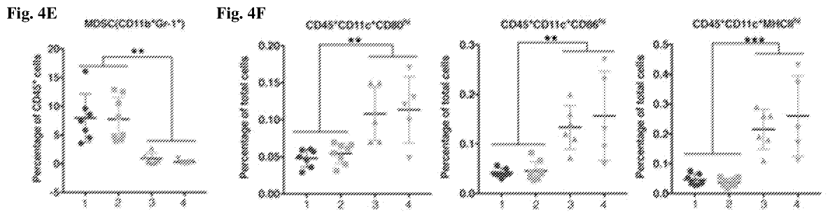

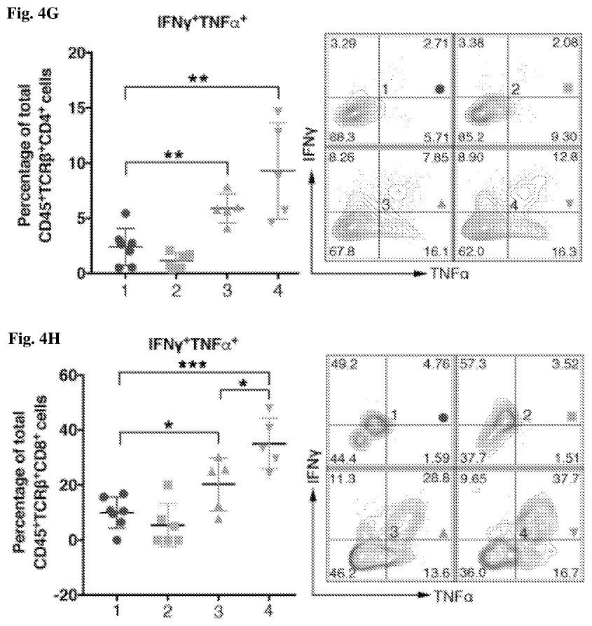

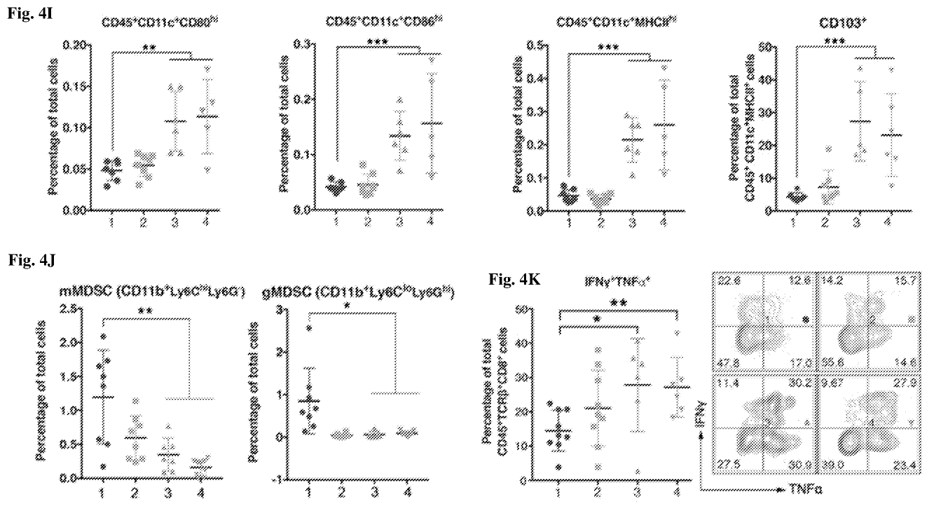

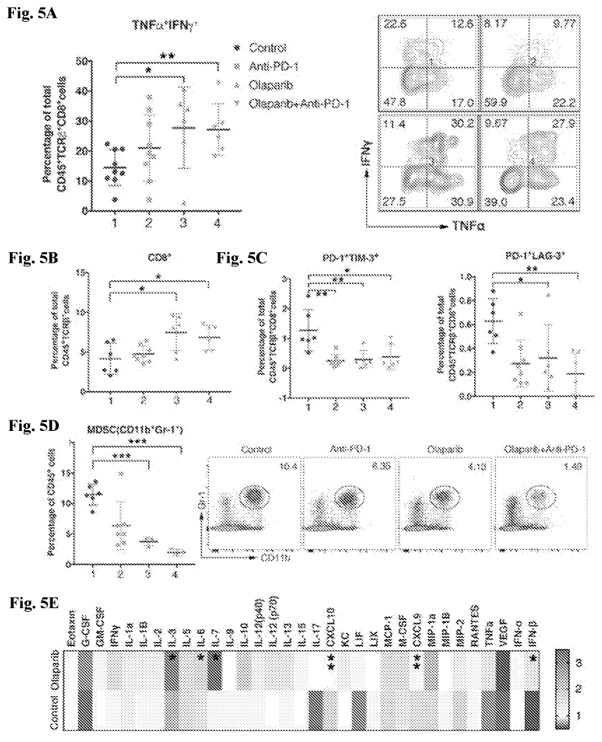

Oliparib provokes robust intratumoral and systemic immune response in Brac1-deficient ovarian tumors. An antitumor immune response elicited by olaparib in Brca1-deficient tumors prompted the study to assess tumor infiltrating immune cells in PBM-bearing mice upon treatment. To determine the immune response in the tumor microenvironment upon olaparib treatment, tumor-infiltrating immune cells from PBM-bearing mice treated with olaparib, PD-1 antibody alone, or their combination, were analyzed. Increased immune cell (CD45+) infiltration into tumor was observed upon olaparib, but not PD-1 antibody treatment (FIGS. 2G and 3A). Further analysis revealed that olaparib treatment alone significantly increased the number of intratumoral CD4+ and CD8+ T cells (FIGS. 3C, 3D, and 4A), but also reduced expression of PD-1/TIM-3 and PD-1/Lag3 co-inhibitory receptors on CD8+ T cells (FIG. 3E). Increase of effector CD8+ T cells was also observed in the malignant ascites of the peritoneal cavity of PBM-bearing mice treated with olaparib (FIG. 3F).

Analysis of functional activity of these T cells revealed that olaparib treatment reduced the exhausted state of the T cells as measured by co-expression of PD-1/TIM-3 and PD-1/Lag3 (FIG. 4B). While intratumoral CD8+ T cells in mice treated with olaparib alone or with both olaparib and anti-PD-1 Ab displayed similar levels of surface PD1 and TIM3 expression, analysis of effector cytokine production by these CD8+ T cells revealed that blockade of immune checkpoint by anti-PD-1 Ab in addition to olaparib treatment led to the increased production of IFN gamma and TNF alpha by CD8+ T cells, indicating enhancement of CD8+ T cell functional activity (FIGS. 4C-4D). This observation is believed to explain the elevated effect of combined treatment on inhibition of tumor progression compared to olaparib single treatment (FIGS. 1B-1C). The frequency of intratumoral FoxP3+ Treg was not changed in mice treated with olaparib, anti-PD-1 Ab, or olaparib+anti-PD-1 Ab (FIG. 3B). Intratumoral CD4+ and CD8+ T cells have significantly increased production IFNγ and TNFα production upon olaparib treatment, addition of PD-1 antibody led to further increased production of these cytokines in these CD8+ T cells (FIG. 4H). The latter finding explains the observation that combined treatment of olaparib and PD-1 antibody exerted a greater anti-tumor activity compared to olaparib single treatment.

Next, the changes of intratumoral myeloid-derived cells as part of the tumor microenvironment in response to olaparib treatment in PBM-bearing mice were assessed. Enhanced functional activity of CD4+ and CD8+ T cells upon olaparib treatment is believed to be associated with decreased suppressive immune environment that is mainly generated by myeloid cells. Tumor-derived signals can modulate innate immune cells including MDSCs, tumor associated DCs and macrophages during tumor progression and their phenotypic modifications towards pro- or anti-inflammatory cells can be a critical determinant for the level of anti-tumor immune response in ovarian cancer subjected to this therapeutic regimen (Conejo-Garcia et al. (2016) Pharmacol. Ther. 164:97-104). Analysis of myeloid cells within tumor revealed a decrease of intratumoral CD11b⁺Gr1⁺ MDSC cells (FIG. 4E) and an increase of antigen presenting capacity of tumor associated DCs that is evidenced by increased levels of CD80, CD86 and MHC class II expression (FIG. 4F). These phynotypic changes in myeloid cells were dependent on PARP inhibition by Olaparib but not anti-PD-1 treatment in this PBM model.

Analysis of DCs in the tumor microenvironment showed increased levels of CD80, CD86 and MHC class II expression upon olaparib treatment (FIG. 4I), indicating that these tumor-associated DCs have increased antigen presenting capacities upon olaparib treatment. Moreover, CD103+ DCs, a subset of APCs known to be potent stimulators of effector T cell trafficking and priming of T cell immunity (Broz et al. (2014) Cancer Cell 26:938; Salmon et al. (2016) Immunity 44:924-938), were increased at the tumor site upon olaparib treatment (FIG. 4I). Olaparib treatment also reduced the CD11b+Ly6CloLy6Ghi granulocytic myeloid-derived suppressor cells (gMDSC) cells (FIG. 4I), a population of myeloid-derived cells with potent immune suppressive properties that is enriched in the microenvironment of ovarian tumor (Cubillos-Ruiz et al. (2010) Cell Cycle 9:260-268; Cubillos-Ruiz et al. (2015) Cell 161:1527-1538). In contrast, intratumoral immune responses following olaparib treatment were not found in PPM tumor bearing mice upon olaparib alone or in combination with PD-1 blockade (FIGS. 4J and 4K). Taken together, these data indicate that olaparib treatment triggered a robust antitumor immunity in the PBM tumor microenvironment involving both adaptive and innate immune responses with activation of lymphoid and myeloid derived cells.

### Example 4: PARP Inhibition Elicited Systemic Adaptive and Innate Immune Responses in Mice Bearing Brca1-Deficient Tumors

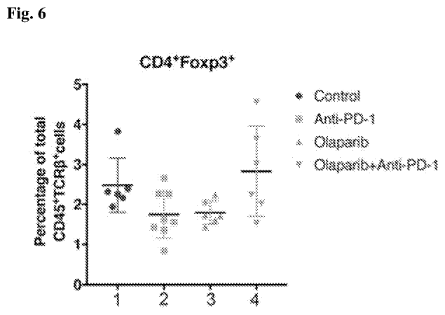

Recent organism-wide assessment of effective anti-tumor immune response secondary to immunotherapy revealed that rejection of less immunogenic tumor requires a sustained systemic immune response that activates a broad immune cell network (Spitzer et al. (2017) Cell 168: 487-502). The systemic immune response to olaparib and its combination with PD-1 blockade in PBM tumor bearing mice was investigated. Analysis of myeloid-derived suppressor cells in the peripheral blood showed that both granulocytic MDSC (gMDSC) and monocytic MDSC (mMDSC) decreased after olaparib and/or PD-1 antibody treatment (FIG. 4J). Analysis of CD8+ T cells in the peripheral blood showed that production of effector cytokines, including IFN gamma and TNF alpha, was enhanced in PBM tumor-bearing mice treated with olaparib (FIG. 4K), which was dependent on PARP inhibition but not on PD-1 blockade (FIG. 5A). Olaparib treatment also significantly increased CD8+ T cells and reduced the expression of PD-1, Tim3, and Lag3 co-inhibitory receptors on CD8+ T cells in the spleens of PBM-bearing mice (FIGS. 5B-5C; FIGS. 3K and 3L). Interestingly, while PD-1 antibody treatment did not change the number of CD8+ T cells, it significantly reduced the expression of PD-1, Tim3, and Lag3 co-inhibitory receptors on CD8+ T cells in the spleens of these mice (FIGS. 3K and 3L). However, this phenotypic change in peripheral CD8+ T cells upon olaparib treatment was to a lesser extent as compared to the changes observed in intratumoral CD8+ T cells (FIGS. 4A-4B and FIGS. 5B-5C). The enhanced immunity in the periphery upon olaparib treatment cannot be attributed to the release from Treg-mediated suppression, since no change in FoxP3+ Treg was observed between these treatment regimens (FIG. 6). On the other hand, the observed systemic immunity upon PARP inhibition could be extended to the myeloid compartment. The number of MDSCs in the spleens was significantly reduced upon olaparib treatment, which was further depressed by the addition of PD-1 antibody (FIG. 5D). The cytokine array analysis of plasma in control- or Olaparib-treated PBM tumor-bearing mice on day 2 of treatment showed changes on the levels of various cytokines (FIG. 5E). These data indicate that PARP inhibition by olaparib elicits both strong intratumoral and systemic adaptive and innate immune responses in mice bearing Brca1-deficient tumors.

### Example 5: Activation of the STING Pathway in Tumor-Associated Antigen Presenting Cells Through Recognition of DNAs Released from BRCA Deficient Tumor

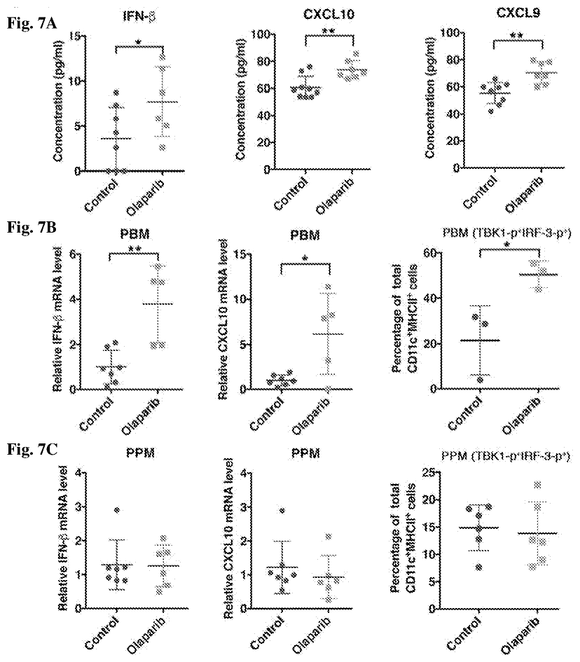

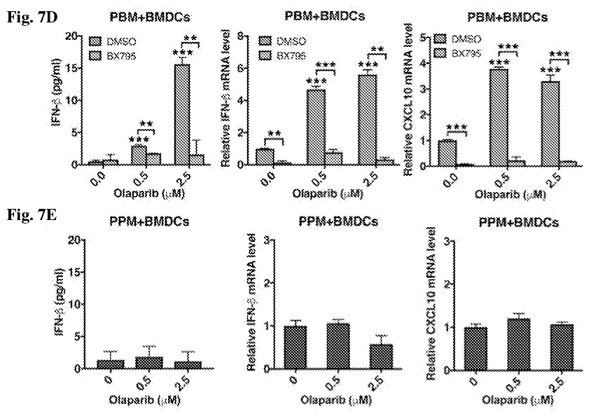

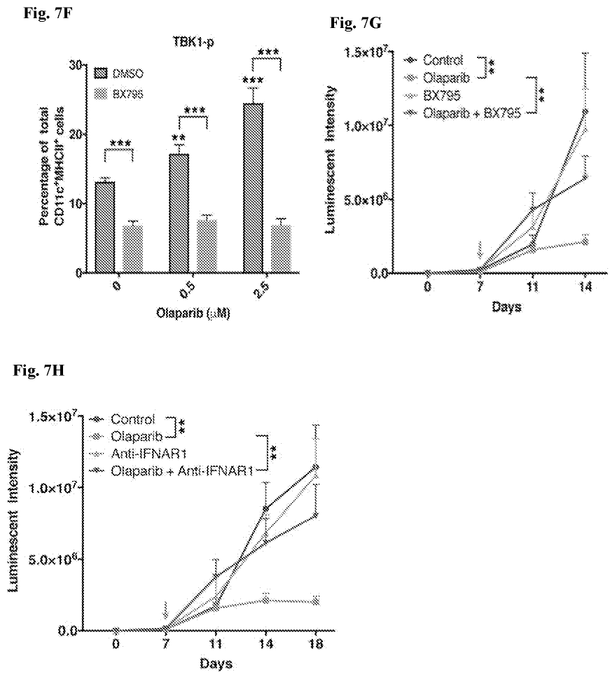

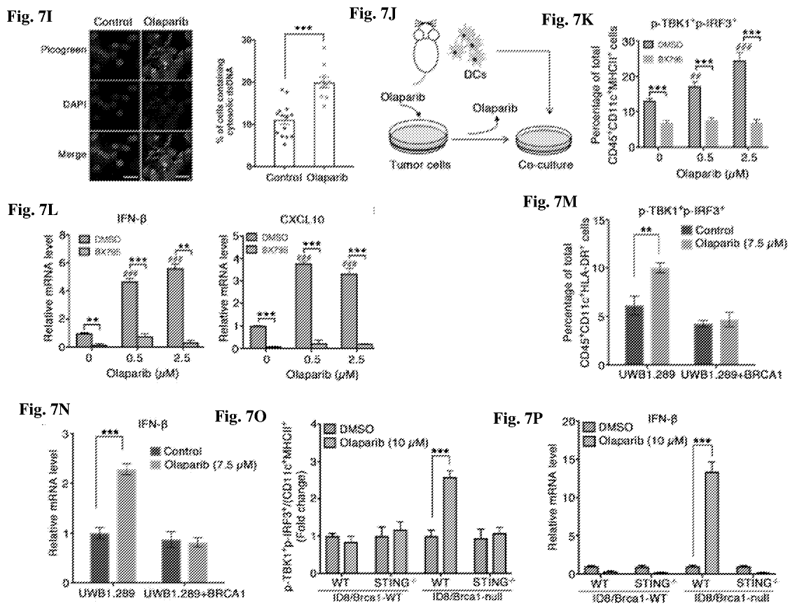

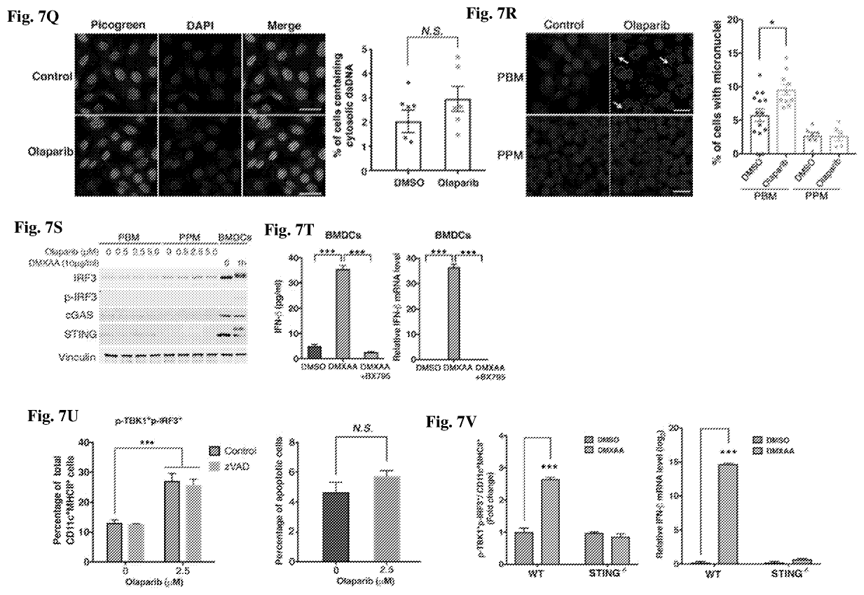

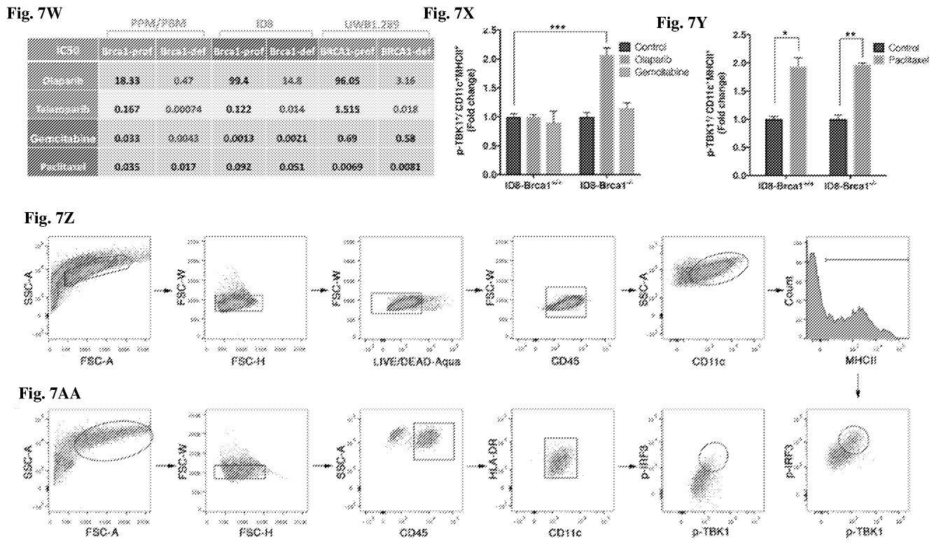

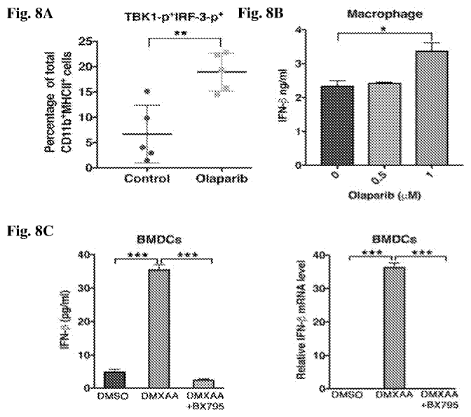

The strong induction of effector T cell response primarily by olaparib treatment indicates that the initial event of cancer cell death resulting from defective DNA repair upon PARP inhibition in BRCA-deficient tumor cells may underlie the promoted immune response in the PBM tumor. Analysis of the cytokine profile in the periphery of PBM-tumor bearing mice after olaparib treatment revealed a significant increase of CXCL9, CXCL10 and IFNβ, which are chemokines and cytokines that indicate activation of innate immune cells including dendritic cells and macrophages, and that have the potential to promote effector T cell migration and priming (FIG. 7A). In line with the significant increase of type I IFN in the periphery, an induction of IFN-β, a type I IFN, and Cxcl10, a type I IFN-stimulated gene (Deng et al. (2014) Immunity 41:843-852; Corrales et al. (2015) Cell Reports 11:1018-1030) in tumor tissues upon olaparib treatment was observed (FIG. 7B). The strong induction of type I IFN in PBM tumors upon PARP inhibition indicated the activation of STING-mediated cytosolic DNA sensing pathway upon olaparib treatment in Brca1-deficient tumors (Parkes et al. (2016) J. Natl. Cancer Inst. 109(1)). Indeed, intratumoral DCs and macrophages have increased phosphorylation of TBK1 and IRF3, the essential signaling intermediaries leading to type I IFN expression, following Olaparib treatment (FIG. 7B and FIG. 8A). This DC phenotype associated with the activation of STING pathway in intratumoral DCs upon olaparib treatment was not observed in PPM tumors (FIG. 7C).

To assess activation of the STING pathway leading to type I IFN production by phagocytes secondary to the sensing of tumor-derived DNA, co-culture experiments were performed using olaparib-treated PBM and PPM tumors cells with bone marrow-derived DCs (BMDCs) or macrophages (BMMs). Significantly enhanced expression of IFN-β and CXCL10 at both mRNA and protein levels was observed in DCs that were co-cultured with olaparib-treated PBM cells compared to DCs with non-treated PBM cells (FIG. 7D). This enhanced IFNβ and CXCL10 expression by DCs was abrogated by an inhibitor of STING pathway, BX795 (FIG. 7D) that specifically inhibited STING-dependent signaling pathway (FIG. 8C). Similar results were observed with BMMs co-cultured with PBM tumors following Olaparib treatment (FIGS. 8A-8B). STING-dependent activation of DCs measured by IFNβ and CXCL10 expression was not observed when DCs were co-cultured with olaparib-treated PPM tumor cells (FIG. 7E). These data indicate that activation of STING pathway in tumor-associated antigen presenting cells through recognition of DNAs released from BRCA-deficient tumor is the underlying mechanism for the enhanced immune response upon PARP inhibition. Olaparib-induced phosphorylation of TBK1 in CD11c dendritic cell population in the same co-culture system of (FIG. 7D) was reduced by BX795 treatment (FIG. 7F). This mechanism was also demonstrated in vivo with BX795 or blocking antibody against IFNAR1 (FIG. 7G and FIG. 7H).

### Example 6: Olaparib Treated Brca1-Deficient Tumor Cells Activate the STING-Pathway in DCs in a Co-Culture System

It has been reported that PARP inhibition induces cell cycle arrest in S/G2, and proliferation inhibition with accumulation of mitotic chromosome bridges and micronuclei formation (Maya-Mendoza et al. (2018) Nature 559:279-284). Recent studies have also reported double-stranded DNA breakage and micronuclei formation after radiotherapy and chemotherapies leading to cGAS-STING signaling pathway-dependent inflammatory responses in tumors (Harding et al. (2017) Nature 548:466-470; Mackenzie et al. (2017) Nature 548:461-465). Therefore, the presence of cytosolic dsDNAs and micronuclei in tumor cells upon olaparib treatment were assessed. As expected, PBM cells, but not PPM cells, have increased cytosolic dsDNAs and micronuclei upon olaparib treatment (FIGS. 7I, 7Q, and 7R).

Similar to numerous tumor cells with defective STING signaling as reported previously (Xia et al. (2016) Cell Rep. 14:282-297; Xia et al. (2016) Cancer Res. 76:6747-6759), the PBM and PPM tumor cells used herein also have low baseline cGAS or STING expression and have little STING signaling activity upon olaparib treatment (FIG. 7S). Mouse bone marrow-derived DCs (BMDCs) have much higher levels of baseline cGAS and STING and activation of the STING signaling when treated with the STING agonist DMXAA (5,6 dimethylxanthenone-4-acetic acid) (Prantner et al. (2012) J. Biol. Chem. 287:39776-3988) (FIG. 7S). DMXAA induced a strong IFN-β production from BMDCs in culture, which was abolished by addition of BX795, an aminopyrimidine that inhibits TBK1/IKKε, and hence inhibits the STING-dependent signaling pathway (Kim et al. (2013) ACS Chem. Biol. 8:1396-1401) (FIG. 7T). Thus, co-culture of olaparib treated tumor cells with BMDCs was performed to determine whether tumor-derived dsDNA can activate the STING signaling in DCs (FIG. 7J). The results presented herein show that BMDCs, when co-cultured with olaparib-treated PBM cells, have markedly increased expression of IFN-β and CXCL10 as well as increased phosphorylation of TBK1 and IRF3 (p-TBK1+p-IRF3+) as compared to BMDCs co-cultured with PBM cells treated with DMSO control (FIGS. 7D and 7J-7L). These effects were abrogated by addition of a STING inhibitor BX795 to the culture (FIGS. 7D and 7J-7L). However, this STING-dependent activation of BMDCs was not observed in the co-culture of BMDCs with PPM tumor cells treated with olaparib (FIG. 7E). Increased levels of p-TBK1+p-IRF3+ and IFN-β were also detected in human DCs co-cultured with BRCA1-deficient human ovarian cancer cells UWB1.289, but not with BRCA1-proficient UWB1.289 cells re-expressing BRCA1, upon olaparib treatment (FIGS. 7M and 7N). Furthermore, it is discovered herein that olaparib treatment at 2.5 uM for 24 hours did not induce substantial cell death in PBM cells, and the addition of apoptosis inhibitor zVAD had little effect on the activation of DCs co-cultured with PBM tumor cells treated with olaparib (FIG. 7U), indicating that dsDNAs produced from viable Brca1-null cells treated with olaparib can activate the STING pathway in DCs.

To further demonstrate the importance of STING signaling in DCs for olaparib induced immune response in Brca1-null tumors, STING-KO mice (Stinggt/gt, C57BL/6J) were employed and confirmed that their BMDCs have defective STING function (FIG. 7V). ID8 cell line, a murine ovarian cancer model in the C57BL/6J background, was also used. Since ID8 cells have wild-type Brca1, a Brca1-null ID8 line was generated by CRISPR/Cas9. Increased p-TBK1+p-IRF3+ and IFN-β were only found in BMDCs derived from wild-type mice, but not from STING-KO mice, when co-cultured with olaparib-treated Brca1-null ID8 (FIGS. 7O and 7P).

It was next examined whether the STING-dependent activation of DCs is specific to PARP inhibition in the context of Brca1-deficiency. IC50 evaluation was performed for a panel of drugs including two PARP inhibitors (olaparib and talazoparib) and two cytotoxic agents (gemcitabine and paclitaxel) on three pairs of Brca1-proficient and Brca1-deficient models (FIG. 7W). In comparison to Brca1-proficient counterparts, all Brca1-deficient lines are much more sensitive to PARP inhibitors (FIG. 7W). In contrast, there are no substantial differences in IC50 values between Brca1-proficient and -deficient cells for gemcitabine or paclitaxel (except PPM and PBM cells treated with gemcitabine). Further analysis also indicated that activation of STING pathway occurred in BMDCs when co-cultured with olaparib treated Brca-deficient tumor cells, but not in gemcitabine-treated tumor cells (FIG. 7X). Interestingly, the phosphorylation of TBK1 and IRF3 were increased in BMDCs when co-cultured with both Brca1-deficient and Brca1 proficient ID8 cells treated with paclitaxel (FIG. 7Y). Together, these results indicate changes specifically caused by PARP inhibition in Brca1-deficient cells.

### Example 7: Materials and Methods for Examples 8-11

a. Tumor Growth and Treatment

All animal experiments described in this study were performed according to the animal protocols approved by the DFCI Institutional Animal Care and Use Committee. Pten^{-/-}; Trp53^{-/-} (termed PP) mammary epithelial tumor cells were derived from syngeneic FVB/N genetically engineered mouse model of breast tumors driven by concurrent loss of PTEN and P53 (K14-Cre/Pten^{L/L}, Trp53^{L/L}). 10⁶ PP cells in 100 μl of 40% Matrigel® Matrix (Corning) in DMEM were injected into the mammary fat pad of FVB/N1 mice. When tumors were about 100-200 mm³ in volume, three injections of 10⁶ of Trp53^{-/-}; Brca1^{-/-}; c-Myc (termed PBM) ovarian surface epithelial tumor cells or PP cells were administered intraturnorally (IT) on day 1, 5, and 9, respectively. Mice were also administered 50 mg/kg/day of olaparib (AZD2281) in 10% DMSO via intraperitoneal injection (IP) from day 1. Olaparib was prepared by diluting 100 mg/ml stocks in DMSO with 10% of 2-hydroxyl-propyl-β-cyclodextrine (Sigma) in PBS. In another cohort of PP tumor, two doses of DMXAA (Sigma, 500 μg/dose) resuspended in 7.5% of NaHCO₃ or vehicle control were injected IT over a one-week period. Tumor volume was calculated using the formula: V=0.52×length×width×width.

b. Preparation of Single Cell Suspensions from Tumors

The resected mouse tumors were mechanically dissociated with surgical scissors and digested with collagenase buffer (8 mL DMEM, 0.1 mL 1M HEPES, 0.5 mL FBS, 0.2 mL Pen/Strep, 1 mL 10× collagenase/hyaluronidase (Stemcell technologies), 0.2 mL 1 mg/mL DNase I (Stemcell technologies)) for 45 min. in a 37° C. shaking incubator. After enzymatic dissociation, samples were transferred to ice and added with DMEM with 10% FBS to stop the reaction. The tumor suspension was then strained using a 70 μm cell strainer (Becton Dickinson) and washed with FACS buffer (1% BSA in PBS) and centrifuged at 1,500 r.p.m. for 5 min. at 4° C. (same centrifugation parameters were used throughout for flow cytometry). Red blood cells were lysed with lysis buffer (4 mL NH4C1+1 mL PBS with 2% calf serum) followed by washing with FACS buffer. The samples were then resuspended in FACS buffer and kept on ice throughout the rest of the staining process.

c. Flow Cytometry Analysis

Samples were first incubated with LIVE/DEAD® Fixable Aqua Dead Cell Stain (Life Technologies, Catalog # L34965 and used at 1:1000 dilution) for 30 min. and washed with FACS buffer. Cells were then incubated with anti-mouse CD16/32 (BioLegend, clone 93) for 20 min. on ice to block non-specific Fc receptor binding. Following Fc block, appropriate antibodies were added to samples at a 1:100 dilution, unless otherwise indicated, for 30 min, on ice and washed with FACS buffer. A fixation and permeabilization buffer set (eBioscience, Cat #00-5523-00) was applied before antibody incubation in order to stain intracellular targets. Antibodies used in the studies include CD45 (BioLegend, clone 30-F11), CD11b (BioLegend, clone M1/70), CD11c (BioLegend, clone BM8), F4/80 (BioLegend, clone BM8), MHCII (BioLegend, clone M5/114.15.2), CD206 (BioLegend, C068C2), Phospho-IRF-3 (Ser396) (D601M) Rabbit mAb (Cell Signaling Technology, Cat #10327, used at 1:50), Phospho-TBK1/NAK (Ser172) (D52C2) Rabbit mAb (Cell Signaling Technology, Cat #13498, used at 1:50 dilution). Flow cytometry was performed on a Fortessa™ HTS cell analyzer (BD Biosciences) at the DFCI flow cytomnetry core facility, and all data were analyzed using FlowJo software.

### Example 8: Exogenous HR-Deficient Cells in Combination with PARP Inhibition Produced Potent Antitumor Potential

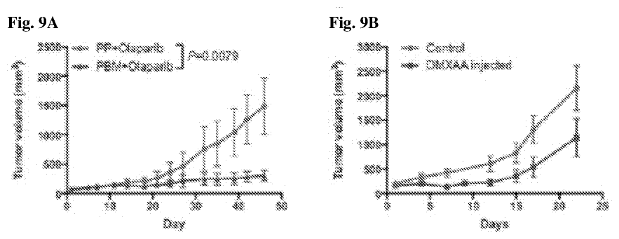

To exploit the concept of using cancer cells to treat cancer by engaging a STING-dependent antitumor immunity upon PARP inhibition, PBM cells (derived from syngeneic FVB GEMM of ovarian cancer driven by co-loss of p53 and Brca1 and overexpression of c-Myc, termed PBM, described in Example 2 above) were used as exogenous HR-deficient cells, and PP cells (derived from syngeneic FVB GEMM of breast tumors driven by concurrent loss of p53 and Pten, termed PP tumors) as exogenous HR-proficient cells. PBM and PP cells were introduced into established PP tumors in FVB mice via intratumoral injection. Notably, PP tumors in mice that received PBM HR-deficient tumor cells, but not PP HR-proficient cells, had retarded tumor growth upon olaparib treatment (FIG. 9A). The PBM+olaparib treatment appeared to be more effective than DMXAA (5,6-dimethylxanthenone-4-acetic acid, also known as Vadimezan or ASA404), which is a potent murine STING agonist (Prantner et al. (2012) J. Biol. Chem. 287:39776-3988; Kim et al. (2013) ACS Chem. Biol. 8:1396-1401), on suppressing the PP tumor growth (FIGS. 9A-9B). These data indicate that exogenous HR-deficient cells in combination with PARP inhibition produced potent antitumor potential.

### Example 9: Activation of the STING Pathway in Tumors Harboring HR-Deficient Cells Upon Olaparib Treatment

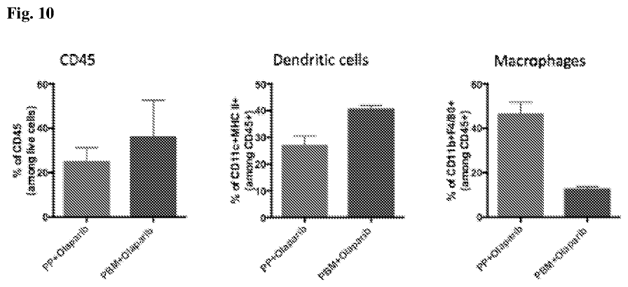

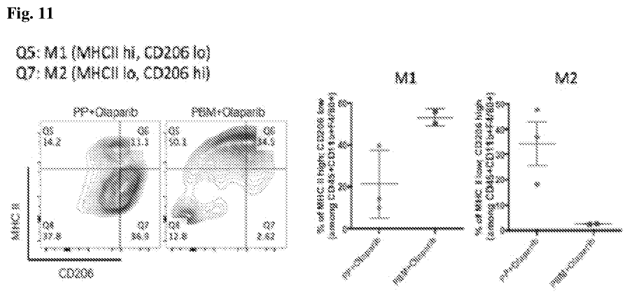

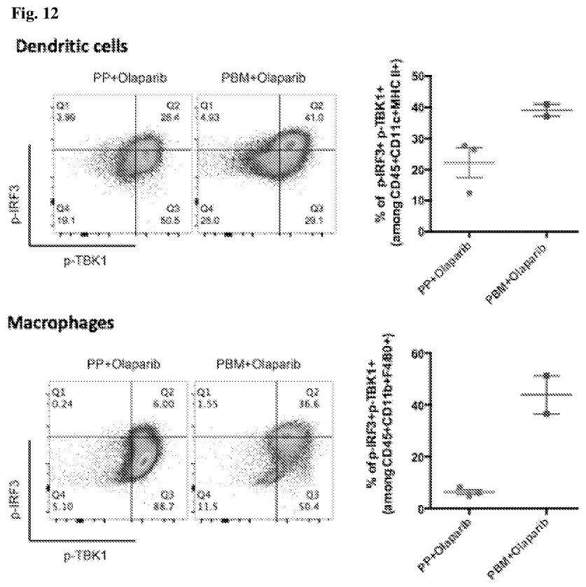

To determine the immune responses in these PP tumors received exogenous PP or PBM cells treated with olaparib, tumors were harvested on day 12 for analysis. Introducing PBM cells in the PP tumors led to the increased leukocytes and dendritic cells (DCs) in the tumor microenvironment following olaparib treatment compared to introducing PP cells to the tumors (FIG. 10, left and middle panels). Interestingly, while the tumor-associated macrophages greatly reduced in tumors with PBM+olaparib (FIG. 10, right panel), these tumors have much increased M1 population and reduced M2 population in comparison to tumors with PP+olaparib (FIG. 11). Further analysis revealed that intratumoral DCs and macrophages displayed marked increased phosphorylation of TBK1 and IRF3 in tumors with PBM+olaparib (FIG. 12), indicating activation of the STING pathway in tumors harboring HR-deficient cells upon olaparib treatment.

### Example 10: Activation of the STING Pathway is Required for the Antitumor Efficacy of Olaparib in Brca1 Deficient Tumors

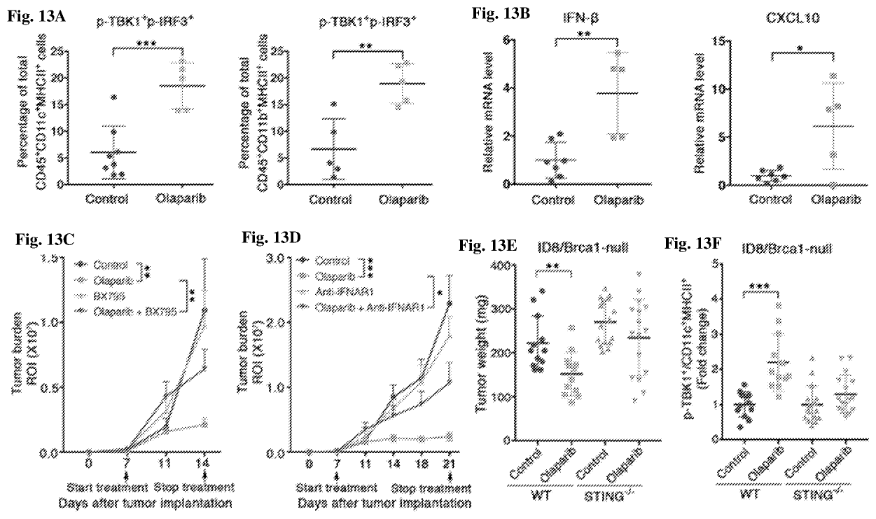

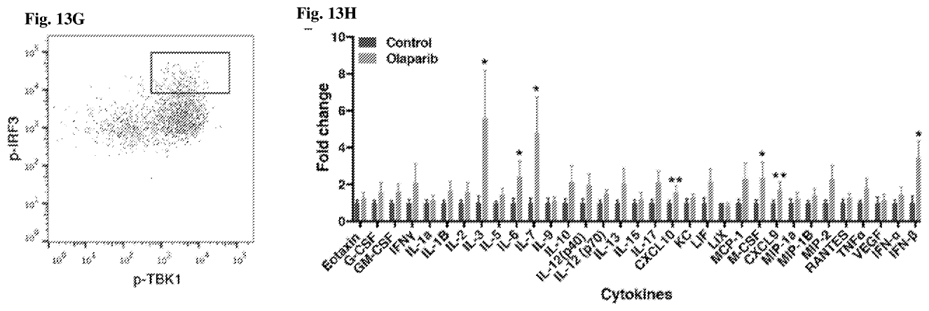

To investigate whether the STING-mediated immune response is important for the antitumor efficacy of olaparib in Brca1-deficient tumors in vivo, it was first demonstrated herein an increased pTBK1+p-IRF3+ in intratumoral APCs, including DCs and macrophages, of PBM tumors after olaparib treatment (FIGS. 13A and 13G). Increased expression of IFN-β and CXCL10 were also detected in olaparib treated PBM tumors (FIG. 13B). Moreover, cytokine profiling of the sera collected from PBM tumor-bearing mice treated with olaparib revealed increased levels of multiple cytokines, including CXCL9 and CXCL10, as well as IFN-β (FIG. 13H). In contrast, these hallmarks of STING pathway activation (Tanaka and Chen (2012) Science signaling 5:ra20; Wu and Chen (2014) Annual review of immunology 32:461-488) were not observed in Brca1-proficient PPM tumors upon olaparib treatment (FIG. S4C, D). These data demonstrate that activation of the STING pathway in response to PARP inhibition is specific to Brca1-deficient tumors.

Next, the importance of STING-mediated immunity in antitumor activity of PARP inhibition on Brca1-deficient tumors with BX795 and a blocking antibody against IFNAR1 was assessed. Both BX795 and anti-IFNAR1 antibody attenuated the antitumor activity of olaparib on PBM tumors (FIG. 13C and FIG. 13D), indicating that activation of the STING pathway and type I IFN responses are important for the antitumor activity of olaparib in Brca-deficient tumors. To further demonstrate that the STING pathway is important for PARP inhibition-induced antitumor immunity in Brca1 deficient tumors, Brca1-null ID8 cells were subcutaneously injected into wild-type (WT) and STING-KO mice. The results demonstrate that olaparib significantly inhibited the tumor growth in WT mice, but had little effect on tumor growth in STING-KO hosts (FIG. 13E). An increased abundance of intratumoral p-TBK1+ DCs was only detected in ID8/Brca1-null tumors from WT host, but not from STING-KO mice, after olaparib treatment (FIG. 13F). Together, the data demonstrate that activation of the STING pathway in tumor-associated APCs through recognition of DNA fragments from Brca1-deficient tumors is an underlying mechanism for immune-mediated antitumor activity of PARP inhibition.

Based on the foregoing, a new mechanism of PARP inhibitor action in vivo is described involving coordinated activation of robust local and systemic anti-tumor immune responses and dependent on underlying HR deficiency. Unlike previously known DNA repair specific mechanisms of PARP inhibitor activity which were unraveled in vitro mostly using cell line models, the new mechanistic descriptions presented herein were identified utilizing a pair of genetically engineered mouse models of HGSOCs, a Brca1-deficient (PBM model) and a Brca1-wildtype (PPM, HR proficient model). Specifically, it is demonstrated herein that olaparib treatment increased the number of intratumoral CD4+ and CD8+ T cells and significantly increased the production of IFNγ and TNFα from these cells. This increased activation of intratumoral CD4+ and CD8+ T cells was associated with increased recruitment of DCs displaying potent antigen presentation capacity in the tumor microenvironment, and was accompanied by significantly reduced MDSCs in the tumor tissue, the spleen and the blood. All these immune responses elicited by olaparib were specific to the HR-deficiency context as they were observed in the Brca1-deficient tumors but not in the HR proficient tumors. Mechanistically, these coordinated robust local and systemic anti-tumor immune responses following PARP inhibition occurred via activation of the STING pathway in APCs and were mediated by sensing of tumor-derived DNA. The relative dependence of this new mode of action for PARP inhibition via on STING-mediated immunity was highlighted by the fact that STING-pathway inhibition or STING knockout significantly attenuated the anti-tumor activity of olaparib in Brca1-deficient tumors.

Importantly, Brca1-deficient PBM tumors treated with olaparib alone had significantly increased expression of the immune-inhibitory ligand PD-L1 on tumor cells both in vivo and in vitro, and addition of immune checkpoint blockade by PD-1 antibody to olaparib resulted in sustained suppression of PBM tumors and extended survival compared to olaparib alone where delayed tumor growth was observed. These data indicate that, while olaparib is effective in treating HR-deficient PBM tumors, activation of the PD-1/PD-L1 immune inhibitory pathway limits its activity, and that this limitation can be overcome by incorporating an anti-PD-1 antibody into the treatment regimen. This observation has important clinical implications because although patients with HR deficient HGSOCs initially respond to PARP inhibitors, a substantial fraction of these patients eventually develop progressive tumors, which represents a significant problem in the clinic. The results presented herein demonstrate that addition of PD-1 blockade prolongs the activity of PARP inhibition by overcoming the increased expression of PD-L1 on tumor cells that occurs after treatment with PARP inhibitors alone. The results presented herein also highlight the importance of investigating the STING pathway as a biomarker of efficacy in the clinic, especially in clinical trials evaluating combinations of PARP-inhibitors with immune checkpoint inhibitors targeting, for example, the PD-1 pathway.

### Example 11: Cancer Vaccine Compositions and Uses Thereof in Representative Embodiments

As described herein, the cancer vaccine compositions of the present invention embody numerous forms. The following provided several representative embodiments. In one embodiment, DNA repair deficient tumor cells, such as those that are HR-deficient and such as murine cells, are engineered from different types of cancer (including solid and hematological cancer) with different genetic makeups (e.g., PP, 4T1, EMT-6, GL261, MC38 and Pan02, etc.) by inactivating Brca1/2 to transform them into HR-deficient cells. Independent approaches are taken to inactivate Brca1/2 in cells, such as siRNA (small interference RNA)-mediated downregulation, CRSPR-mediated deletion or chemical-mediated degradation. Subsequent experiments are carried out to assess the antitumor immunity of these engineered HR-deficient cells in tumor-bearing mice with PARP inhibitors, e.g., olaparib, rucaparib or niraparib, alone and in combination with the immune check point blockade (anti-PD1 or anti-PD-L1 antibody, or anti-CD47 antibody). In order to eliminate any possibility that injected engineered tumor cells give rise to new neoplastic lesions, the engineered tumor cells can be irradiated to suppress cell proliferation before injection. HR-deficient cells can also be treated with PARP inhibitors in vitro before introducing them in vivo. PARP inhibitor administration in vivo can be avoided in this case. Alternatively, non-cancerous cells can be engineered to harbor Brca1/2 deficiencies for this purpose.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A cancer vaccine comprising DNA repair-deficient cancer cells, wherein the cancer cells are contacted with a PARP inhibitor to induce DNA breaks.

2. The cancer vaccine of claim 1, wherein the cancer cells have reduced copy number, amount, and/or activity of one or more DNA damage checkpoints and/or DNA damage repair genes.

3. The cancer vaccine of claim 2, wherein one or more DNA damage checkpoints are selected from the group consisting of Brca1, Brca2, Chk1, Chk2, ATM, ATR, Cdc25C, and Nbs1.

4. The cancer vaccine of claim 2, wherein the one or more DNA damage repair genes are selected from the group consisting of non-homologous end joining (NHEJ), microhomology-mediated end joining (MMEJ), and homologous recombination pathway genes.

5. The cancer vaccine of claim 4, wherein the one or more DNA damage repair genes are selected from the group consisting of BLM, MSH2, MSH6, MLH1, PMS2, MRE11, DNA Ligase IV, TP53BP1, RAD51, RAD51L1, RAD51C, RAD51L3, DMC1, XRCC2, XRCC3, XRCC4, NBS1, RAD50, GADD45, RFC2, XRCC6, POLD2, PCNA, RPA1, RPA2, ERCC3, UNG, ERCC5, MLH1, LIG1, NBN, MSH6, POLD4, RFC5, DDB2, POLD1, FANCG, POLB, XRCC1, MPG, RFC2, ERCC1, TDG, FANCA, RFC4, RFC3, APEX2, RAD1, BRCA1, FEN1, MLH3, MGMT, RAD51, XRCC4, RECQL, ERCC8, FANCC, OGG1, MRE11A, RAD52, WRN, XPA, BLM, OGG1, MSH3, POLE2, RAD51C, LIG4, ERCC6, LIG3, RAD17, XRCC2, MUTYH, RFC1, BRCA2, RAD50, DDB1, XRCC5, PARP1, POLE3, RFC1, RAD50, XPC, MSH2, RPA3, MBD4, NTHL1, PMS2/PMS2CL, RAD51C, UNG2, APEX1, ERCC4, RAD1, RECQL5, MSH5, RECQL, RAD52, XRCC4, RAD17, MSH3, MRE11A, MSH6, and RECQL5.

6. The cancer vaccine of any one of claims 1-5, wherein the copy number, amount, and/or activity of one or more DNA damage checkpoints and/or DNA damage repair genes are reduced by contacting the cancer cells with a small molecule inhibitor, CRISPR guide RNA (gRNA), RNA interfering agent, antisense oligonucleotide, peptide or peptidomimetic inhibitor, aptamer, antibody, or intrabody.

7. The cancer vaccine of claim 6, wherein the RNA interfering agent is a small interfering RNA (siRNA), CRISPR RNA (crRNA), a CRISPR guide RNA (gRNA), a small hairpin RNA (shRNA), a microRNA (miRNA), or a piwi-interacting RNA (piRNA).

8. The cancer vaccine of claim 6, wherein the antibody and/or intrabody, or antigen binding fragment thereof, specifically binds to one or more DNA damage checkpoints and/or DNA damage repair genes.

9. The cancer vaccine of claim 8, wherein the antibody and/or intrabody, or antigen binding fragment thereof, is murine, chimeric, humanized, composite, or human.

10. The cancer vaccine of claim 8 or 9, wherein the antibody and/or intrabody, or antigen binding fragment thereof, comprises an effector domain, comprises an Fc domain, and/or is selected from the group consisting of Fv, Fav, F(ab′)2, Fab′, dsFv, scFv, sc(Fv)2, and diabodies fragments.

11. The cancer vaccine of any one of claims 1-10, wherein the DNA breaks comprise double-strand DNA breaks or single-strand DNA breaks.

12. The cancer vaccine of any one of claims 1-11, wherein the PARP inhibitor is selected from the group consisting of olaparib, rucaparib, niraparib, veliparib (ABT-888), talazoparib (BMN 673), iniparib (BSI-201), E7449, INO-1001, AZD2461, ME0328, TNKS49, TNKS22, JW55, PJ34, INO-1001, WIKI4, NU 1025, DR 2313, BYK 49187, BYK 204165, MK-4827, UPF 1069, A-966492, 4-HQN, EB47, MK-4827 hydrochloride, MK-4827 tosylate, and MK-4827 racemate.

13. The cancer vaccine of any one of claims 1-12, wherein the cancer cells are contacted with the PARP inhibitor alone in vitro, in vivo, and/or ex vivo, optionally wherein the cancer cells are contacted with the PARP inhibitor in combination with an immune checkpoint blockade in vitro, in vivo or ex vivo.

14. The cancer vaccine of claim 13, wherein the cancer cells are contacted with the PARP inhibitor in vitro or ex vivo.

15. The cancer vaccine of claim 13, wherein the cancer cells are administered to a subject, wherein the PARP inhibitor is administered to the subject to thereby contact the cancer cells in vivo.

16. The cancer vaccine of claim 15, wherein the PARP inhibitor is administered before, after, or concurrently with administration of the cancer cells.

17. The cancer vaccine of any one of claims 1-16, wherein the cancer cells are derived from a solid or hematological cancer.

18. The cancer vaccine of claim 17, wherein the cancer cells are derived from a cancer cell line.

19. The cancer vaccine of claim 18, wherein the cancer cell line is selected from the group consisting of PP, 4T1, EMT-6, GL261, MC38, Pan02, CT26, KLN205, Lewis Lung, Madison 109, MBT-2, Colon26, CT26, A20, E.G7-OVA, B16F10, ClondmanS91, and Renca.

20. The cancer vaccine of any one of claims 1-19, wherein the cancer cells are ovarian cancer cells, optionally wherein the ovarian cancer cells are high grade serous ovarian cancer cells (HGSOC).

21. The cancer vaccine of any one of claims 1-19, wherein the cancer cells are derived from an ovarian cancer driven by co-loss of p53 and Brca1 and overexpression of c-Myc or a breast cancer driven by co-loss of p53 and Brca1.

22. The cancer vaccine of any one of claims 1-21, wherein the cancer vaccine reduces the number of proliferating cells in the cancer and/or reduces the volume or size of a tumor comprising the cancer cells.

23. The cancer vaccine of any one of claims 1-22, wherein the cancer vaccine increases the amount of CD45⁺ immune cells infiltrating a tumor.

24. The cancer vaccine of any one of claims 1-23, wherein cancer vaccine increases the amount of intra-tumoral and peripheral IFNg⁺ TNFa⁺ CD8⁺ T cells, CD103+CD8⁺ T cells, and/or IFNg⁺ TNFa⁺ CD4⁺ T cells.

25. The cancer vaccine of any one of claims 1-24, wherein the cancer vaccine decreases CD11b⁺Gr1⁺ myeloid derived suppressor cells (MDSCs) in tumor tissue and/or spleen.

26. The cancer vaccine of any one of claims 1-25, wherein the cancer vaccine increases intra-tumoral dendritic cells (DCs) that display an enhanced antigen presentation capacity.

27. The cancer vaccine of claim 26, wherein the cancer vaccine increases amount and/or activity of CD80, CD86, CD103, CD8a, MHC class I, and/or MHC class II on intra-tumoral DCs.

28. The cancer vaccine of any one of claims 1-27, wherein the cancer vaccine reduces intra-tumoral macrophages.

29. The cancer vaccine of claim 28, wherein the cancer vaccine increases intra-tumoral M1 macrophage cells and reduces intra-tumoral M2 macrophage cells.

30. The cancer vaccine of any one of claims 26-29, wherein the cancer vaccine activates STING-dependent cytosolic DNA sensing pathway in the intra-tumoral DCs and/or macrophages.

31. The cancer vaccine of claim 30, wherein the cancer vaccine increases type I IFN in peripheral immune cells.

32. The cancer vaccine of claim 31, wherein the cancer vaccine induces expression of IFN-alpha, IFN-beta, Cxcl9, Cxcl10, IL-3, IL-6, IL-7, M-CSF, TNFa, IFNg, Ccl2, Ccl5, GM-CSF, and Ccl20 in the peripheral blood and/or tumor tissues.

33. The cancer vaccine of any one of claims 26-32, wherein the cancer vaccine increases dimerization and phosphorylation of STING, dimerization and nuclear translocation of IRF3, activation of IKK, phosphorylation of IkB family of inhibitors of the transcription factor NF-kB, phosphorylation of TBK1, IRF3, JAK1/2 and/or STAT1/2, and/or activates JAK/STAT pathway in the intra-tumoral DCs and/or macrophages.

34. The cancer vaccine of any one of claims 1-33, wherein the cancer cells are non-replicative.

35. The cancer vaccine of claim 34, wherein the cancer cells are non-replicative due to irradiation.

36. The cancer vaccine of any one of claims 1-35, wherein the cancer vaccine is administered to a subject in combination with an immunotherapy and/or cancer therapy, optionally wherein the immunotherapy and/or cancer therapy is administered before, after, or concurrently with the cancer vaccine.

37. The cancer vaccine of claim 36, wherein the immunotherapy is cell-based.

38. The cancer vaccine of claim 36, wherein the immunotherapy comprises a cancer vaccine and/or virus.

39. The cancer vaccine of claim 36, wherein the immunotherapy inhibits an immune checkpoint.

40. The cancer vaccine of claim 39, wherein the immune checkpoint is selected from the group consisting of CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRPalpha (CD47), CD48, 2B4 (CD244), B7.1, B7.2, ILT-2, ILT-4, TIGIT, HHLA2, butyrophilins, and A2aR.

41. The cancer vaccine of claim 40, wherein the immune checkpoint is PD1, PD-L1, or CD47.

42. The cancer vaccine of claim 36, wherein the cancer therapy is selected from the group consisting of radiation, a radiosensitizer, and a chemotherapy.

43. A method of treating a subject afflicted with a cancer comprising administering to the subject a therapeutically effective amount of a cancer vaccine comprising DNA repair-deficient cancer cells, wherein the cancer cells are contacted with a PARP inhibitor to induce DNA breaks.

44. The method of claim 43, wherein the cancer cells have reduced copy number, amount, and/or activity of one or more DNA damage checkpoints and/or the DNA damage repair genes.

45. The method of claim 44, wherein one or more DNA damage checkpoints are selected from the group consisting of Brca1, Brca2, Chk1, Chk2, ATM, ATR, Cdc25C, and Nbs1.

46. The method of claim 44, wherein the one or more DNA damage repair genes are selected from the group consisting of non-homologous end joining (NHEJ), microhomology-mediated end joining (MMEJ), and homologous recombination pathway genes.

47. The method of claim 46, wherein the one or more DNA damage repair genes are selected from the group consisting of BLM, MSH2, MSH6, MLH1, PMS2, MRE11, DNA Ligase IV, TP53BP1, RAD51, RAD51L1, RAD51C, RAD51L3, DMC1, XRCC2, XRCC3, XRCC4, NBS1, RAD50, GADD45, RFC2, XRCC6, POLD2, PCNA, RPA1, RPA2, ERCC3, UNG, ERCC5, MLH1, LIG1, NBN, MSH6, POLD4, RFC5, DDB2, POLD1, FANCG, POLB, XRCC1, MPG, RFC2, ERCC1, TDG, FANCA, RFC4, RFC3, APEX2, RAD1, BRCA1, FEN1, MLH3, MGMT, RAD51, XRCC4, RECQL, ERCC8, FANCC, OGG1, MRE11A, RAD52, WRN, XPA, BLM, OGG1, MSH3, POLE2, RAD51C, LIG4, ERCC6, LIG3, RAD17, XRCC2, MUTYH, RFC1, BRCA2, RAD50, DDB1, XRCC5, PARP1, POLE3, RFC1, RAD50, XPC, MSH2, RPA3, MBD4, NTHL1, PMS2/PMS2CL, RAD51C, UNG2, APEX1, ERCC4, RAD1, RECQL5, MSH5, RECQL, RAD52, XRCC4, RAD17, MSH3, MRE11A, MSH6, and RECQL5.

48. The method of any one of claims 43-47, wherein the copy number, amount, and/or activity of one or more DNA damage checkpoints and/or DNA damage repair genes are reduced by contacting the cancer cells with a small molecule inhibitor, CRISPR guide RNA (gRNA), RNA interfering agent, antisense oligonucleotide, peptide or peptidomimetic inhibitor, aptamer, antibody, or intrabody.

49. The method of claim 48, wherein the RNA interfering agent is a small interfering RNA (siRNA), CRISPR RNA (crRNA), a CRISPR guide RNA (gRNA), a small hairpin RNA (shRNA), a microRNA (miRNA), or a piwi-interacting RNA (piRNA).

50. The method of claim 48, wherein the antibody and/or intrabody, or antigen binding fragment thereof, specifically binds to one or more DNA damage checkpoints and/or DNA damage repair genes.

51. The method of claim 50, wherein the antibody and/or intrabody, or antigen binding fragment thereof, is murine, chimeric, humanized, composite, or human.

52. The method of claim 50 or 51, wherein the antibody and/or intrabody, or antigen binding fragment thereof, comprises an effector domain, comprises an Fc domain, and/or is selected from the group consisting of Fv, Fav, F(ab′)2, Fab′, dsFv, scFv, sc(Fv)2, and diabodies fragments.

53. The method of any one of claims 43-52, wherein the DNA breaks comprise double-strand DNA breaks or single-strand DNA breaks.

54. The method of any one of claims 43-53, wherein the PARP inhibitor is selected from the group consisting of olaparib, rucaparib, niraparib, veliparib (ABT-888), talazoparib (BMN 673), iniparib (BSI-201), E7449, INO-1001, AZD2461, ME0328, TNKS49, TNKS22, JW55, PJ34, INO-1001, WIKI4, NU 1025, DR 2313, BYK 49187, BYK 204165, MK-4827, UPF 1069, A-966492, 4-HQN, EB47, MK-4827 hydrochloride, MK-4827 tosylate, and MK-4827 racemate.

55. The method of any one of claims 43-54, wherein the cancer cells are contacted with the PARP inhibitor alone in vitro, in vivo, and/or ex vivo, optionally wherein the cancer cells are contacted with the PARP inhibitor in combination with an immune checkpoint blockade in vitro, in vivo or ex vivo.

56. The cancer vaccine of claim 55, wherein the cancer cells are contacted with the PARP inhibitor in vitro or ex vivo.

57. The cancer vaccine of claim 55, wherein the cancer cells are administered to a subject, wherein the PARP inhibitor is administered to the subject to thereby contact the cancer cells in vivo.

58. The cancer vaccine of claim 57, wherein the PARP inhibitor is administered before, after, or concurrently with administration of the cancer cells.

59. The method of any one of claims 43-58, wherein the cancer cells are derived from a solid or hematological cancer.

60. The cancer vaccine of claim 59, wherein the cancer cells are derived from a cancer cell line.

61. The cancer vaccine of claim 60, wherein the cancer cell line is selected from the group consisting of PP, 4T1, EMT-6, GL261, MC38, Pan02, CT26, KLN205, Lewis Lung, Madison 109, MBT-2, Colon26, CT26, A20, E.G7-OVA, B16F10, ClondmanS91, and Renca.

62. The cancer vaccine of any one of claims 43-61, wherein the cancer cells are ovarian cancer cells, optionally wherein the ovarian cancer cells are high grade serous ovarian cancer cells (HGSOC).

63. The cancer vaccine of any one of claims 43-61, wherein the cancer cells are derived from an ovarian cancer driven by co-loss of p53 and Brca1 and overexpression of c-Myc or a breast cancer driven by co-loss of p53 and Brca1.

64. The method of any one of claims 43-63, wherein the cancer cells are derived from a cancer that is the same type as the cancer treated with the cancer vaccine.

65. The method of any one of claims 43-63, wherein the cancer cells are derived from a cancer that is a different type from the cancer treated with the cancer vaccine.

66. The method of any one of claims 43-63, wherein the cancer cells are derived from the subject who is treated with the cancer vaccine.

67. The method of any one of claims 43-63, wherein the cancer cells are derived from a different subject who is not treated with the cancer vaccine.

68. The method of any one of claims 43-68, wherein the cancer vaccine reduces the number of proliferating cells in the cancer and/or reduces the volume or size of a tumor comprising the cancer cells.

69. The method of any one of claims 43-69, wherein cancer vaccine increases the amount of CD45⁺ immune cells infiltrating a tumor.

70. The method of any one of claims 43-70, wherein cancer vaccine increases the amount of intra-tumoral and peripheral IFNg⁺ TNFa⁺ CD8⁺ T cells, CD103-CD8⁺ T cells, and/or IFNg⁺ TNFa⁺ CD4⁺ T cells.

71. The method of any one of claims 43-71, wherein the cancer vaccine decreases CD11b⁺Gr1⁺ myeloid derived suppressor cells (MDSCs) in the tumor tissue and/or spleen.

72. The method of any one of claims 43-72, wherein the cancer vaccine increases intra-tumoral dendritic cells (DCs) that display an enhanced antigen presenting capacity.

73. The method of claim 72, wherein the cancer vaccine increases amount and/or activity of CD80, CD86, CD103, CD8a, MHC class I, and/or MHC class II on intra-tumoral DCs.

74. The method of any one of claims 43-73, wherein the cancer vaccine reduces intra-tumoral macrophages.

75. The method of claim 74, wherein the cancer vaccine increases intra-tumoral M1 macrophage cells and reduces M2 intra-tumoral macrophage cells.

76. The method of any one of claims 43-75, wherein the cancer vaccine activates STING-dependent cytosolic DNA sensing pathway in the intra-tumoral DCs and/or macrophages.

77. The method of claim 76, wherein the cancer vaccine increases type I IFN in the peripheral immune cells.

78. The method of claim 77, wherein the cancer vaccine induces expression of IFN-alpha, IFN-beta, Cxcl9, Cxcl10, IL-3, L-6, IL-7, M-CSF, TNFa, IFNg, Ccl2, Ccl5, GM-CSF, and Ccl20 in the peripheral blood and/or tumor tissues.

79. The method of claim 72-78, wherein the cancer vaccine increases dimerization and phosphorylation of STING, dimerization and nuclear translocation of IRF3, activation of IKK, phosphorylation of IkB family of inhibitors of the transcription factor NF-kB, phosphorylation of TBK1, IRF3, JAK1/2 and/or STAT1/2, and/or activation of JAK/STAT pathway in the intra-tumoral DCs and/or macrophages.

80. The method of any one of claims 43-79, wherein the cancer cells are non-replicative.

81. The method of claim 80, wherein the cancer cells are non-replicative due to irradiation.

82. The method of any one of claims 43-81, wherein the method further comprising administering to the subject an immunotherapy and/or cancer therapy, optionally wherein the immunotherapy and/or cancer therapy is administered before, after, or concurrently with the cancer vaccine.

83. The method of claim 82, wherein the immunotherapy is cell-based.

84. The method of claim 82, wherein the immunotherapy comprises a cancer vaccine and/or virus.

85. The method of claim 82, wherein the immunotherapy inhibits an immune checkpoint.

86. The method of claim 85, wherein the immune checkpoint is selected from the group consisting of CTLA-4, PD-1, VISTA, B7-H2, B7-H3, PD-L1, B7-H4, B7-H6, ICOS, HVEM, PD-L2, CD160, gp49B, PIR-B, KIR family receptors, TIM-1, TIM-3, TIM-4, LAG-3, GITR, 4-IBB, OX-40, BTLA, SIRPalpha (CD47), CD48, 2B4 (CD244), B7.1, B7.2, ILT-2, ILT-4, TIGIT, HHLA2, butyrophilins, and A2aR.

87. The method of claim 86, wherein the immune checkpoint is PD1, PD-L1, or CD47.

88. The method of claim 87, wherein the cancer therapy is selected from the group consisting of radiation, a radiosensitizer, and a chemotherapy.

89. A method of assessing the efficacy of the cancer vaccine of claim 1 for treating a subject afflicted with a cancer, comprising:
a) detecting in a subject sample at a first point in time the number of proliferating cells in the cancer and/or the volume or size of a tumor comprising the cancer cells;
b) repeating step a) during at least one subsequent point in time after administration of the cancer vaccine; and
c) comparing the number of proliferating cells in the cancer and/or the volume or size of a tumor comprising the cancer cells detected in steps a) and b), wherein the absence of, or a significant decrease in number of proliferating cells in the cancer and/or the volume or size of a tumor comprising the cancer cells in the subsequent sample as compared to the number and/or the volume or size in the sample at the first point in time, indicates that the cancer vaccine treats cancer in the subject.

90. The method of claim 89, wherein between the first point in time and the subsequent point in time, the subject has undergone treatment, completed treatment, and/or is in remission for the cancer.

91. The method of claim 89 or 90, wherein the first and/or at least one subsequent sample is selected from the group consisting of ex vivo and in vivo samples.

92. The method of any one of claims 89-91, wherein the first and/or at least one subsequent sample is a portion of a single sample or pooled samples obtained from the subject.

93. The method of any one of claims 89-92, wherein the sample comprises cells, serum, peripheral lymphoid organs, and/or intratumoral tissue obtained from the subject.

94. The method of any one of claims 89-93, further comprising determining responsiveness to the agent by measuring at least one criteria selected from the group consisting of clinical benefit rate, survival until mortality, pathological complete response, semi-quantitative measures of pathologic response, clinical complete remission, clinical partial remission, clinical stable disease, recurrence-free survival, metastasis free survival, disease free survival, circulating tumor cell decrease, circulating marker response, and RECIST criteria.

95. The method of any one of claims 43-94, wherein the cancer vaccine is administered in a pharmaceutically acceptable formulation.

96. The method of any one of claims 43-95, wherein the step of administering occurs in vivo, ex vivo, or in vitro.

97. The method of any one of claims 1-96, wherein the cancer vaccine is administered to the subject intratumorally or subcutaneously.

98. The method of any one of claims 1-97, wherein the subject is an animal model of the cancer, optionally wherein the animal model is a mouse model.

99. The method of any one of claims 1-97, wherein the subject is a mammal.

100. The method of claim 99, wherein the mammal is a mouse or a human.

101. The method of claim 100, wherein the mammal is a human.
